(19) United States
(12) Patent Application Publication
(10) Pub. No.: US 2005/0085436 A1
(43) Pub. Date: Apr. 21, 2005

(54) Method to treat conditions associated with insulin signalling dysregulation

(76) Inventors: Hao Li, Carlisle, MA (US); Jing Ma, Scotch Plains, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/887,553

(22) Filed: Jul. 8, 2004

Related U.S. Application Data
(60) Provisional application No. 60/485,883, filed on Jul. 8, 2003.

(51) Int. Cl. A61K 48/00

(52) U.S. Cl. 514/44

(57) ABSTRACT

The invention discloses a method to identify proteins involved in the ISP. The invention also discloses suitable targets for the development of new therapeutics to treat, prevent or ameliorate pathological conditions associated with dysregulation of the ISP. The invention also relates to methods to treat, prevent or ameliorate said conditions and pharmaceutical compositions therefor, as well as to a method to identify compounds with therapeutic usefulness to treat pathological conditions associated with dysregulation of the ISP.

### BACKGROUND OF INVENTION

This invention relates to newly identified proteins involved in the insulin signaling npathway, methods for identifying compounds useful to treat pathological conditions associated with dysregulation of the insulin signaling pathway (ISP), as well as to methods and pharmaceutical compositions to treat, prevent or ameliorate conditions associated with dysregulation of the ISP.

### FIELD OF INVENTION

Using Drosophila as a model system, Applicants herein disclose a method to identify proteins involved in the ISP. Employing said method, Applicants have discovered and describe herein several new proteins involved in the ISP. It is contemplated herein that these proteins and the genes encoding said proteins may serve as drug targets for the development of therapeutics to treat, prevent or ameliorate diabetes and other pathological conditions associated with dysregulation of the ISP.

### SUMMARY OF THE INVENTION

The instant application discloses a method to employ transgenic Drosophila to identify proteins involved in the ISP. Human homologs of the Drosophila genes identified according to this method are suitable targets for the development of new therapeutics to treat, prevent or ameliorate pathological conditions associated with the dysregulation of the ISP. Thus, in one aspect the invention relates to a method to identify modulators useful to treat, prevent or ameliorate said conditions:
(a) assaying for the ability of a candidate modulator to modulate the biochemical function of a protein selected from the group consisting of those disclosed in Table 13 or 25 and/or modulate expression of said protein and which can further include;
(b) assaying for the ability of an identified modulator to reverse the pathological effects observed in animal models of pathological conditions associated with the dysregulation of the ISP and/or in clinical studies with subjects with said conditions.

In another aspect, the invention relates to a method to treat, prevent or ameliorate pathological conditions associated with the dysregulation of the ISP, comprising administering to a subject in need thereof an effective amount of a modulator of a protein selected from the group consisting of those disclosed in Table 13 or 25, wherein said modulator, e.g., inhibits or enhances the biochemical function of said protein. In a further embodiment, the modulator comprises antibodies and/or peptide mimetics to said protein or fragments thereof, wherein said antibodies and peptide mimetics can inhibit the biochemical function of said protein in said subject.

In another embodiment the modulator inhibits or enhances the expression of a protein selected from the group consisting of those disclosed in Table 13 or 25. In a further embodiment, the modulator comprises any one or more substances selected from the group consisting of antisense oligonucleotides, triple-helix DNA, ribozymes, ribonucleic acid (RNA) aptamers, siRNA and double- or single-stranded RNA wherein said substances are designed to inhibit expression of said protein.

In another aspect, the invention relates to a method to treat, prevent or ameliorate pathological conditions associated with dysregulation of the ISP comprising administering to a subject in need thereof a pharmaceutical composition comprising an effective amount of a modulator of a protein selected from the group consisting of those disclosed in Table 13 or 25. In various embodiments, said pharmaceutical composition comprises antibodies and/or peptide mimetics to said protein or fragments thereof, wherein said antibodies and peptide mimetics can inhibit the biochemical function of said protein in said subject and/or any one or more substances selected from the group consisting of antisense oligonucleotides, triple-helix DNA, ribozymes, RNA aptamers, siRNA and double- or single-stranded RNA wherein said substances are designed to inhibit expression of said protein.

In another aspect, the invention relates to a pharmaceutical composition comprising a modulator of a protein selected from the group consisting of those disclosed in Table 13 or 25 in an amount effective to treat, prevent or ameliorate a pathological condition associated with dysregulation of the ISP, including Type II diabetes and the Type A syndrome of insulin resistance, in a subject in need thereof. In one embodiment, said modulator may, e.g., inhibit or enhance the biochemical functions of said protein. In a further embodiment said modulator comprises antibodies and/or peptide mimetics to said protein or fragments thereof, wherein said antibodies or peptide mimetics can, e.g., inhibit the biochemical functions of said protein.

In a further embodiment, said pharmaceutical composition comprises a modulator which may, e.g., inhibit or enhance expression of said protein. In a further embodiment, said modulator comprises any one or more substances selected from the group consisting of antisense oligonucleotides, triple helix DNA, ribozymes, RNA aptamers, siRNA or double or single-stranded RNA directed to a nucleic acid sequence of said protein wherein said substances are designed to inhibit expression of said protein.

In another aspect, the invention relates to a method to diagnose subjects suffering from pathological conditions associated with dysregulation of the ISP who may be suitable candidates for treatment with modulators to a protein selected from the group consisting of those disclosed in Table 13 or 25 comprising detecting levels of any one or more of said proteins in a biological sample from said subject wherein subjects with altered levels compared to controls would be suitable candidates for modulator treatment.

In another aspect, the invention relates to a method to diagnose subjects suffering from pathological conditions associated with dysregulation of the ISP who may be suitable candidates for treatment with modulators to a protein selected from the group consisting of those disclosed in Table 13 or 25 comprising assaying messenger RNA (mRNA) levels of any one or more of said proteins in a biological sample from said subject wherein subjects with altered levels compared to controls would be suitable candidates for modulator treatment.

In yet another aspect, there is provided a method to treat, prevent or ameliorate pathological conditions associated with dysregulation of the ISP comprising:
(a) assaying for mRNA and/or protein levels of a protein selected from the group consisting of those disclosed in Table 13 or 25 in a subject; and
(b) administering to a subject with altered levels of mRNA and/or protein levels compared to controls a modulator to said protein in an amount sufficient to treat, prevent or ameliorate the pathological effects of said condition.

In particular apects, said modulator inhibits or enhances the biochemical function of said protein or expression of said protein.

In yet another aspect of the present invention, there are provided assay methods and diagnostic kits comprising the components necessary to detect mRNA levels or protein levels of any one or more proteins selected from the group consisting of:
(a) those disclosed in Table 13 or 25 in a biological sample, said kit comprising, e.g., polynucleotides encoding any one or more proteins selected from the group consisting of those disclosed in Table 13 or 25;
(b) nucleotide sequences complementary to said protein; and
(c) any one or more of said proteins, or fragments thereof or antibodies or peptide mimetics that bind to any one or more of said proteins, or to fragments thereof.

In a preferred embodiment, such kits also comprise instructions detailing the procedures by which the kit components are to be used.

The present invention also pertains to the use of a modulator to a protein selected from the group consisting of those disclosed in Table 13 or 25 in the manufacture of a medicament for the treatment, prevention or amelioration of pathological conditions associated with dysregulation of the ISP. In one embodiment, said modulator comprises any one or more substances selected from the group consisting of antisense oligonucleotides, triple-helix DNA, ribozymes, RNA aptamer, siRNA and double- or single-stranded RNA wherein said substances are designed to inhibit expression of said protein. In yet a further embodiment, said modulator comprises one or more antibodies and/or peptide mimetics to said protein or fragments thereof, wherein said antibodies and peptide mimetics or fragments thereof can, e.g., inhibit the biochemical function of said protein.

The invention also pertains to a modulator to a protein selected from the group consisting of those disclosed in Table 13 or 25 for use as a pharmaceutical. In one embodiment, said modulator comprises any one or more substances selected from the group consisting of antisense oligonucleotides, triple-helix DNA, ribozymes, RNA aptamer, siRNA and double- or single-stranded RNA wherein said substances are designed to inhibit expression of said protein. In yet a further embodiment, said modulator comprises one or more antibodies and/or peptide mimetics to said protein or fragments thereof, wherein said antibodies and mimetics or fragments thereof can, e.g., inhibit the biochemical functions of said protein.

In another aspect, the invention also pertains to a method to identify Drosophila proteins involved in the ISP, said method comprising:
(a) providing a transgenic fly whose genome comprises a DNA sequence encoding a polypeptide comprising the dominant negative PI3K catalytic subunit Dp110^{D954A}, said DNA sequence operably linked to a tissue specific expression control sequence and expressing said DNA sequence, wherein expression of said DNA sequence results in said fly displaying a transgenic phenotype compared to controls;
(b) crossing said transgenic fly with a fly containing a mutation in a known or predicted gene; and
(c) screening progeny of said crosses for flies that carry said DNA sequence and said mutation and display modified expression of the transgenic phenotype as compared to appropriate controls.

In one embodiment, said DNA sequence encodes Dp110^{D954A} and said tissue specific expression control sequence comprises the eye-specific enhancer ey-Gal4 and expression of said DNA sequence results in said fly displaying the “small eye” phenotype.

In another aspect, the invention pertains to gene regulatory elements, such as promoters, enhancers, inducers or inhibitors of expression of Drosophila proteins selected from the group consisting of those disclosed in Table 13 or 25 and methods of identifying such gene regulatory elements. In general such sequence features are readily identified using computational tools known in the art. Such gene regulatory elements are useful for a variety of purposes, e.g., control of heterologous gene expression, target for identifying gene activity modulating compounds, and are particularly claimed as fragments of the nucleic acid sequences provided herein.

In another aspect, the invention pertains to methods for screening test compounds which modulate transcription of the Drosophila proteins described in Table 13 or 25 by:
(a) contacting a host cell in which the Table 13 or 25 proteins disclosed herein are operably-linked to a reporter gene with a test medium containing the test compound under conditions which allow for expression of the reporter gene;
(b) measuring the expression of the reporter gene in the presence of the test medium;
(c) contacting the host with a control medium which does not contain the test compound but is otherwise identical to the test medium in (a), under conditions identical to those used in (a);
(d) measuring the expression of reporter gene in the presence of the control medium; and
(e) relating the difference in expression between (b) and (d) to the ability of the test compound to regulate the activity of the protein.

In a particular embodiment, the invention relates to a method to identify drug targets for the development of therapeutics to treat, prevent or ameliorate pathological conditions associated with dysregulation of the ISP said method comprising identifying the human homologs of the Drosophila proteins identified according to the method discussed above.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE FIGURES

FIG. 1 depicts the genetic crosses performed to produce the transgenic Drosophila disclosed herein.

### DETAILED DESCRIPTION OF THE INVENTION

All patent applications, patents and literature references cited herein are hereby incorporated by reference in their entirety.

In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, 2^{nd} Edition, Cold Spring Harbor Laboratory Press, Cold Spring Harbor, N.Y. (1989); DNA Cloning: A Practical Approach, Vols. I and II, Glover, ed. (1985); Oligonucleotide Synthesis, Gait, ed. (1984); Nucleic Acid Hybridization, Hames and Higgins (1985); Transcription and Translation, Hames and Higgins, eds. (1984); Animal Cell Culture, Freshney, ed. (1986); Immobilized Cells and Enzymes, IRL Press (1986); Methods in Enzymology, Perbal, ed., Academic Press, Inc. (1984); Gene Transfer Vectors for Mammalian Cells, Miller and Calos, eds., Cold Spring Harbor Laboratory Press, Cold Spring Harbor, N.Y. (1987); and Methods in Enzymology, Vols. 154 and 155, Wu and Grossman and Wu, eds., respectively. Well-known Drosophila molecular genetics techniques can be found, e.g., in Robert, Drosophila, A Practical Approach, IRL Press, Washington, D.C. (1986).

Abbreviations used in the following description include:
IRS insulin receptor substrate
PI3K phosphoinositide 3-kinase
PDK 3′-phosphoinositide-dependent protein kinases
PTEN phosphatase and tensin homolog deleted from chromosome 10
PKB protein kinase B, also known as Akt1

Descriptions of flystocks can be found in the Flybase database at http://flybase.bio.indiana.edu.

Stock centers referred to herein include Bloomington and Szeged stock centers which are located at Bloomington, Ind. and Szeged, Hungary, respectively.

As used herein and in the appended claims, the singular forms “a”, “an” and “the” include plural reference unless the context clearly dictates otherwise. Thus, e.g., reference to “the antibody” is a reference to one or more antibodies and equivalents thereof known to those skilled in the art, and so forth.

“Nucleic acid sequence”, as used herein, refers to an oligonucleotide, nucleotide or polynucleotide, and fragments or portions thereof, and to DNA or RNA of genomic or synthetic origin that may be single- or double-stranded, and represent the sense or antisense strand.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative” is sometimes used in reference to the antisense strand, and “positive” is sometimes used in reference to the sense strand.

“cDNA” refers to DNA that is complementary to a portion of mRNA sequence and is generally synthesized from an mRNA preparation using reverse transcriptase.

As contemplated herein, antisense oligonucleotides, triple helix-DNA, RNA aptamers, ribozymes, siRNA and double- or single-stranded RNA are directed to a nucleic acid sequence such that the nucleotide sequence chosen will produce gene-specific inhibition of gene expression. For example, knowledge of a nucleotide sequence may be used to design an antisense molecule which gives strongest hybridization to the mRNA. Similarly, ribozymes can be synthesized to recognize specific nucleotide sequences of a gene and cleave it. See Cech, JAMA, Vol. 260, p. 3030 (1988). Techniques for the design of such molecules for use in targeted inhibition of gene expression is well-known to one of skill in the art.

The individual proteins/polypeptides referred to herein include any and all forms of these proteins including, but not limited to, partial forms, isoforms, variants, precursor forms, the full-length protein, fusion proteins containing the sequence or fragments of any of the above, from human or any other species. Protein homologs or orthologs which would be apparent to one of skill in the art are included in this definition. It is also contemplated that the term refers to proteins isolated from naturally-occurring sources of any species, such as genomic DNA libraries, as well as genetically-engineered host cells comprising expression systems, or produced by chemical synthesis using, for instance, automated peptide synthesizers or a combination of such methods. Means for isolating and preparing such polypeptides are well-understood in the art.

The term “sample”, as used herein, is used in its broadest sense. A biological sample from a subject may comprise blood, urine, brain tissue, primary cell lines, immortilized cell lines, or other biological material with which protein activity or gene expression may be assayed. A biological sample may include, e.g., blood, tumors or other specimens from which total RNA may be purified for gene expression profiling using, e.g., conventional glass chip microarray technologies, such as Affymetrix chips, RT-PCR or other conventional methods.

As used herein, the term “antibody” refers to intact molecules, as well as fragments thereof, such as Fa, F(ab′)₂ and Fv, which are capable of binding the epitopic determinant. Antibodies that bind specific polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein. Commonly used carriers that are chemically coupled to peptides include bovine serum albumin and thyroglobulin. The coupled peptide is then used to immunize an animal, e.g., a mouse, goat, chicken, rat or rabbit.

The term “humanized antibody”, as used herein, refers to antibody molecules in which amino acids have been replaced in the non-antigen binding regions in order to more closely resemble a human antibody, while still retaining the original binding ability.

A peptide mimetic is a synthetically-derived peptide or non-peptide agent created based on a knowledge of the critical residues of a subject polypeptide which can mimic normal polypeptide function. Peptide mimetics can disrupt binding of a polypeptide to its receptor or to other proteins and thus interfere with the normal function of a polypeptide.

A “therapeutically-effective amount” is the amount of drug sufficient to treat, prevent or ameliorate pathological conditions associated with dysregulation of the ISP.

A “transgenic” organism, as used herein, refers to an organism that has had extra genetic material inserted into its genome. As used herein, a “transgenic fly” includes embryonic, larval and adult forms of Drosophila that contain a DNA sequence from the same or another organism randomly inserted into their genome. Although Drosophila melanogaster is preferred, it is contemplated that any fly of the genus Drosophila may be used in the present invention.

As used herein, “ectopic” expression of the transgene refers to expression of the transgene in a tissue or cell or at a specific developmental stage where it is not normally expressed.

As used herein, “phenotype” refers to the observable physical or biochemical characteristics of an organism as determined by both genetic makeup and environmental influences.

The term “transcription factor” refers to any protein required to inititate or regulate transcription in eukaryotes. For example, the eye-specific promoter GMR is a binding site for the eye-specific transcription factor. See Moses and Rubin, GM Genes Dev, Vol. 5, No. 4, pp. 583-593 (1991).

“UAS” region, as used herein, refers to an up-stream activating sequence recognized by the GAL-4 transcriptional activator.

As used herein, a “control” fly refers to a larva or fly that is of the same genotype as larvae or flies used in the methods of the present invention except that the control larva or fly does not carry the mutation being tested for modification of phenotype.

As used herein, a “transformation vector” is a modified transposable element used with the transposable element technique to mediate integration of a piece of DNA in the genome of the organism and is familiar to one of skill in the art.

As used herein, “elevated transcription of mRNA” refers to a greater amount of mRNA transcribed from the natural endogenous gene encoding a protein, e.g., a human protein set forth in Table 13 or 25, compared to control levels. Elevated mRNA levels of a protein, e.g., a human protein disclosed on Table 13 or 25, may be present in a tissue or cell of an individual suffering from a pathological condition associated with dysregulation of the ISP compared to levels in a subject not suffering from said condition. In particular, levels in a subject suffering from said condition may be at least about 2 times, preferably at least about 5 times, more preferably at least about 10 times, most preferably at least about 100 times the amount of mRNA found in corresponding tissues in humans who do not suffer from said condition. Such elevated level of mRNA may eventually lead to increased levels of protein translated from such mRNA in an individual suffering from a pathological condition associated with dysregulation of the ISP as compared to levels in a healthy individual.

As used herein, a “Drosophila transformation vector” is a DNA plasmid that contains transposable element sequences and can mediate integration of a piece of DNA in the genome of the organism. This technology is familiar to one of skill in the art.

As used herein, the “small eye phenotype” is characterized by reduced cell size in the eye tissue compared to appropriate controls. See Leevers et al., EMBO J, Vol. 15, No. 23, pp. 6584-6594 (1996).

A “host cell”, as used herein, refers to a prokaryotic or eukaryotic cell that contains heterologous DNA that has been introduced into the cell by any means, e.g., electroporation, calcium phosphate precipitation, microinjection, transformation, viral infection and the like.

“Heterologous”, as used herein, means “of different natural origin” or represent a non-natural state. For example, if a host cell is transformed with a DNA or gene derived from another organism, particularly from another species, that gene is heterologous with respect to that host cell and also with respect to descendants of the host cell which carry that gene. Similarly, heterologous refers to a nucleotide sequence derived from and inserted into the same natural, original cell type, but which is present in a non-natural state, e.g., a different copy number, or under the control of different regulatory elements.

A “vector” molecule is a nucleic acid molecule into which heterologous nucleic acid may be inserted which can then be introduced into an appropriate host cell. Vectors preferably have one or more origin of replication, and one or more site into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes”.

“Plasmids” generally are designated herein by a lower case “p” preceded and/or followed by capital letters and/or numbers, in accordance with standard naming conventions that are familiar to those of skill in the art. Starting plasmids disclosed herein are either commercially available, publicly available on an unrestricted basis, or can be constructed from available plasmids by routine application of well-known, published procedures. Many plasmids and other cloning and expression vectors that can be used in accordance with the present invention are well-known and readily-available to those of skill in the art. Moreover, those of skill readily may construct any number of other plasmids suitable for use in the invention. The properties, construction and use of such plasmids, as well as other vectors, in the present invention will be readily-apparent to those of skill from the present disclosure.

The term “isolated” means that the material is removed from its original environment, e.g., the natural environment if it is naturally-occurring. For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

As used herein, the terms “transcriptional control sequence” or “expression control sequence” refer to DNA sequences, such as initiator sequences, enhancer sequences and promoter sequences, which induce, repress or otherwise control the transcription of a protein encoding nucleic acid sequences to which they are operably-linked. They may be tissue-specific and developmental stage-specific.

A “human transcriptional control sequence” is a transcriptional control sequence normally found associated with the human gene encoding a polypeptide set forth in Table 13 or 25 of the present invention as it is found in the respective human chromosome.

A “non-human transcriptional control sequence” is any transcriptional control sequence not found in the human genome.

The term “polypeptide” is used interchangeably herein with the terms “polypeptides” and “protein(s)”. As generally referred to herein, a protein or gene selected from the group consisting of those disclosed in Table 13 or 25 refers to the human protein or gene and its Drosophila homolog.

A “chemical derivative” of a protein set forth in Table 13 or 25 of the invention is a polypeptide that contains additional chemical moieties not normally a part of the molecule. Such moieties may improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed, e.g., in Remington's Pharmaceutical Sciences, 16^{th} edition, Mack Publishing Co., Easton, Pa. (1980).

The ability of a substance to “modulate” a protein set forth in Table 13 or 25, i.e., “a modulator of a protein selected from the group consisting of those disclosed in Table 13 or 25” includes, but is not limited to, the ability of a substance to inhibit the activity of said protein and/or inhibit the gene expression of said protein. Such modulation could also involve affecting the ability of other proteins to interact with said protein, e.g., related regulatory proteins or proteins that are modified by said protein.

The term “agonist”, as used herein, refers to a molecule, i.e., modulator, which directly or indirectly may modulate a polypeptide, e.g., a polypeptide set forth in Table 13 or 25, and which increase the biological activity of said polypeptide. Agonists may include proteins, nucleic acids, carbohydrates or other molecules. A modulator that enhances gene transcription or the biochemical function of a protein is something that increases transcription or stimulates the biochemical properties or activity of said protein, respectively.

The terms “antagonist” or “inhibitor” as used herein, refer to a molecule, i.e., modulator, which directly or indirectly may modulate a polypeptide, e.g., a polypeptide set forth in Table 13 or 25, which blocks or inhibits the biological activity of said polypeptide. Antagonists and inhibitors may include proteins, nucleic acids, carbohydrates or other molecules. A modulator that inhibits gene expression or the biochemical function of a protein is something that reduces gene expression or biological activity of said protein, respectively.

As used herein, “pathological condition associated with dysregulation of the ISP” includes, but is not limited to, diabetes, e.g., Type II diabetes, gestational diabetes and the Type A syndrome of insulin resistance, autoimmune arthritis, asthma, septic shock, lung fibrosis, glomerulonephritis and atherosclerosis.

“In vivo models of a pathological condition associated with dysregulation of the ISP” include those in vivo models of diabetes familiar to those of skill in the art. Such in vivo models include: an association of the common pro12 allele in PPAR-gamma with type-2 diabetes [see Altshuler et al., Nature Genet, pp. 76-80 (2000)], defects in the human insulin receptor gene [see Kadowaki et al., Science, Vol. 240, pp. 787-790 (1988)], defects in the insulin receptor substrate 1 gene in mouse [see Abe et al., J Clin Invest, Vol. 101, pp. 1784-1788 (1998)] and defects in glycogen sythase in humans. See Groop et al., NEJM, Vol. 328, pp. 10-14 (1993).

The present invention further provides non-coding fragments of the nucleic acid molecules provided in Table 13 or 25. Preferred non-coding fragments include, but are not limited to, promoter sequences, enhancer sequences, gene modulating sequences and gene termination sequences. Such fragments are useful in controlling heterologous gene expression and in developing screens to identify gene-modulating agents. A promoter can readily be identified using techniques available in the art as being 5′ to the ATG start site in the genomic sequence provided in Table 13 or 25.

Conventional expression control systems may be used to achieve ectopic expression of proteins of interest, including the Dp110^{D954A} peptide. Such expression may result in the disturbance of biochemical pathways and the generation of altered phenotypes. One such expression control system involves direct fusion of the DNA sequence to expression control sequences of tissue-specifically expressed genes, such as promoters or enhancers. A tissue specific expression control system that may be used is the binary Gal4-transcriptional activation system. See Brand and Perrimon, Development, Vol. 118, pp. 401-415 (1993).

The Gal4 system uses the yeast transcriptional activator Gal4, to drive the expression of a gene of interest in a tissue-specific manner. The Gal4 gene has been randomly inserted into the fly genome, using a conventional transformation system, so that it has come under the control of genomic enhancers that drive expression in a temporal and tissue-specific manner. Individual strains of flies have been established, called “drivers”, that carry those insertions. See Brand and Perrimon (1993), supra.

In the Gal4 system, a gene of interest is cloned into a transformation vector, so that its transcription is under the control of the Upstream Activating Sequence (UAS), the Gal4-responsive element. When a fly strain that carries the UAS gene of interest sequence is crossed to a fly strain that expresses the Gal4 gene under the control of a tissue-specific enhancer, the gene will be expressed in a tissue-specific pattern.

In order to generate phenotypes that are easily visible in adult tissues and can thus be used in genetic screens, Gal4 “drivers” that drive expression in later stages of the fly development may be used in the present invention. Using these drivers, expression would result in possible defects in the wings, the eyes, the legs, different sensory organs and the brain. These “drivers” include, e.g., hsp-Gal4 (heat shock-inducible), apterous-Gal4 (wings), elav-Gal4 (CNS), sevenless-Gal4, eyeless-Gal4 (ey-Gal4) and pGMR-Gal4 (eyes). Descriptions of the Gal4 lines and notes about their specific expression patterns is available in Flybase (http://flybase.bio.indiana.edu).

Various DNA constructs may be used to generate the transgenic Drosophila melanogaster disclosed herein. For example, the construct may contain the Dp110^{D954A} sequence cloned into the pUAST vector [see Brand and Perrimon (1993), supra] which places the UAS sequence up-stream of the transcribed region. Insertion of these constructs into the fly genome may occur through P-element recombination, Hobo element recombination [see Blackman et al., EMBO J., Vol. 8, pp. 211-217 (1989)], homologous recombination [see Rong and Golic, Science, Vol. 288, pp. 2013-2018 (2000)] or other standard techniques known to one of skill in the art.

As discussed above, an ectopically-expressed gene may result in an altered phenotype by disruption of a particular biochemical pathway. Mutations in genes acting in the same biochemical pathway are expected to cause modification of the altered phenotype. Thus, e.g., a transgenic fly carrying both ey-Gal4 and UAS-Dp110^{D954A} can be used to identify genes acting in the ISP by crossing this transgenic fly with a fly containing a mutation in a known or predicted gene; and screening progeny of the crosses for flies that display quantitative or qualitative modification of the altered phenotype of the ey-Gal4/Dp110^{D954A} transgenic fly, as compared to controls. Thus, this system is extremely beneficial for the elucidation of the function of Dp110^{D954A} products, as well as the identification of other genes that directly or indirectly interact with them. Mutations that can be screened include, but are not limited to, loss-of-function alleles of known genes, deletion strains, “enhancer-trap” strains generated by the P-element and gain-of-function mutations generated by random insertions into the Drosophila genome of a Gal4-inducible construct that can activate the ectopic expression of genes in the vicinity of its insertion. It is contemplated herein that genes involved in the ISP (in both Drosophila and humans) can be identified in this manner and these genes can then serve as targets for the development of therapeutics to treat pathological conditions associated with dysregulation in the ISP.

Nucleic acid molecules of the human homologs of the target polypeptides identifed according to the methods of the present invention and disclosed herein may act as target gene antisense molecules, useful, e.g., in target gene regulation and/or as antisense primers in amplification reactions of target gene nucleic acid sequences. Further, such sequences may be used as part of ribozyme and/or triple-helix sequences or as targets for siRNA or double- or single-stranded RNA, which may be employed for gene regulation. Still further, such molecules may be used as components of diagnostic kits as disclosed herein.

In cases where the gene identified using the methods of the present invention is the normal, or wild-type, gene, this gene may be used to isolate mutant alleles of the gene. Such isolation is preferable in processes and disorders which are known or suspected to have a genetic basis. Mutant alleles may be isolated from individuals either known or suspected to have a genotype which contributes to disease symptoms related to pathological conditions associated with dysregulation of the ISP including, but not limited to, conditions, such as Type II diabetes or the Type A syndrome of insulin resistance. See Taylor and Ariogluo, J Basic Clin Physiol Pharmacol, Vol. 9, pp. 419-439 (1998). Mutant alleles and mutant allele products may then be utilized in the diagnostic assay systems described herein.

A cDNA of the mutant gene may be isolated, e.g., by using PCR, a technique which is well-known to those of skill in the art. In this case, the first cDNA strand may be synthesized by hybridizing an oligo-dT oligonucleotide to mRNA isolated from tissue known or suspected to be expressed in an individual putatively carrying the mutant allele, and by extending the new strand with RT. The second strand of the cDNA is then synthesized using an oligonucleotide that hybridizes specifically to the 5′ end of the normal gene. Using these two primers, the product is then amplified via PCR, cloned into a suitable vector and subjected to DNA sequence analysis through methods well-known to those of skill in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof may then be labeled and used as a probe to identify the corresponding mutant allele in the library. The clone containing this gene may then be purified through methods routinely practiced in the art, and subjected to sequence analysis as described above.

Additionally, an expression library can be constructed utilizing DNA isolated from or cDNA synthesized from a tissue known to or suspected of expressing the gene of interest in an individual suspected of or known to carry the mutant allele. In this manner, gene products made by the putatively mutant tissue may be expressed and screened using standard antibody screening techniques in conjunction with antibodies raised against the normal gene product, as described below. For screening techniques, see, e.g., Harlow and Lane, eds., Antibodies: A Laboratory Manual, Cold Spring Harbor Press, Cold Spring Harbor, N.Y. (1988). In cases where the mutation results in an expressed gene product with altered function, e.g., as a result of a missense mutation; a polyclonal set of antibodies are likely to cross-react with the mutant gene product. Library clones detected via their reaction with such labeled antibodies can be purified and subjected to sequence analysis as described above.

In another embodiment, nucleic acids comprising a sequence encoding a polypeptide set forth in Table 13 or 25 or functional derivatives thereof, may be administered to promote normal biological function, e.g., normal insulin mediated signal transduction, by way of gene therapy. Gene therapy refers to therapy performed by the administration of a nucleic acid to a subject. In this embodiment of the invention, the nucleic acid produces its encoded protein that mediates a therapeutic effect by promoting a normal ISP.

Any of the methods for gene therapy available in the art can be used according to the present invention. Exemplary methods are described below.

In a preferred aspect, the therapeutic comprises a nucleic acid for a Table 13 or 25 polypeptide that is part of an expression vector that expresses a Table 13 or 25 protein or fragment or chimeric protein thereof in a suitable host. In particular, such a nucleic acid has a promoter operably-linked to the Table 13 or 25 protein coding region, said promoter being inducible or constitutive, and, optionally, tissue-specific. In another particular embodiment, a nucleic acid molecule is used in which the Table 13 or 25 protein coding sequences and any other desired sequences are flanked by regions that promote homologous recombination at a desired site in the genome, thus providing for intrachromosomal expression of the Table 13 or 25 nucleic acid. See Koller and Smithies, Proc Natl Acad Sci USA, Vol. 86, pp. 8932-8935 (1989); and Zijlstra et al., Nature, Vol. 342, pp. 435-438 (1989).

Delivery of the nucleic acid into a patient may be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, e.g., U.S. Pat. No. 4,980,286 and others mentioned infra), or by direct injection of naked DNA, or by use of microparticle bombardment, e.g., a gene gun; Biolistic, Dupont; or coating with lipids or cell-surface receptors or transfecting agents, encapsulation in liposomes, microparticles or microcapsules, or by administering it in linkage to a peptide which is known to enter the nucleus, by administering it in linkage to a ligand subject to receptor-mediated endocytosis (see e.g., U.S. Pat. Nos. 5,166,320; 5,728,399; 5,874,297; and 6,030,954, all of which are incorporated by reference herein in their entirety) which can be used to target cell types specifically expressing the receptors, etc. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell-specific uptake and expression, by targeting a specific receptor. See, e.g., PCT Publications WO 92/06180, WO 92/22635, WO 92/20316, WO 93/14188 and WO 93/20221. Alternatively, the nucleic acid can be introduced intracellularly and incorporated within host cell DNA for expression, by homologous recombination. See, e.g., U.S. Pat. Nos. 5,413,923, 5,416,260 and 5,574,205; and Zijistra et al. (1989), supra.

In a specific embodiment, a viral vector that contains a nucleic acid encoding a Table 13 or 25 polypeptide is used. For example, a retroviral vector can be used. See, e.g., U.S. Pat. Nos. 5,219,740, 5,604,090 and 5,834,182. These retroviral vectors have been modified to delete retroviral sequences that are not necessary for packaging of the viral genome and integration into host cell DNA. The nucleic acid for the Table 13 or 25 polypeptide to be used in gene therapy is cloned into the vector, which facilitates delivery of the gene into a patient.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Methods for conducting adenovirus-based gene therapy are described in, e.g., U.S. Pat. Nos. 5,824,544, 5,868,040, 5,871,722, 5,880,102, 5,882,877, 5,885,808, 5,932,210, 5,981,225, 5,994,106, 5,994,132, 5,994,134, 6,001,557 and 6,033,8843, all of which are incorporated by reference herein in their entirety.

Adeno-associated virus (AAV) has also been proposed for use in gene therapy. Methods for producing and utilizing AAV are described, e.g., in U.S. Pat. Nos. 5,173,414, 5,252,479, 5,552,311, 5,658,785, 5,763,416, 5,773,289, 5,843,742, 5,869,040, 5,942,496 and 5,948,675, all of which are incorporated by reference herein in their entirety.

Another approach to gene therapy involves transferring a gene to cells in tissue culture by such methods as electroporation, lipofection, calcium phosphate mediated transfection or viral infection. Usually, the method of transfer includes the transfer of a selectable marker to the cells. The cells are then placed under selection to isolate those cells that have taken up and are expressing the transferred gene. Those cells are then delivered to a patient.

The resulting recombinant cells can be delivered to a patient by various methods known in the art. In a preferred embodiment, epithelial cells are injected, e.g., subcutaneously. In another embodiment, recombinant skin cells may be applied as a skin graft onto the patient. Recombinant blood cells, e.g., hematopoietic stem or progenitor cells, are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type and include, but are not limited to, epithelial cells, endothelial cells, keratinocytes, fibroblasts, muscle cells, hepatocytes; blood cells, such as T lymphocytes, B lymphocytes, monocytes, macrophages, neutrophils, eosinophils, megakaryocytes and granulocytes; various stem or progenitor cells, in particular, hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, the nucleic acid of a polypeptide set forth in Table 13 or 25 is introduced into the cells such that it is expressible by the cells or their progeny, and the recombinant cells are then administered in vivo for therapeutic effect. In a specific embodiment, stem or progenitor cells are used. Any stem cells and/or progenitor cells that can be isolated and maintained in vitro can potentially be used in accordance with this embodiment of the present invention. Such stem cells include, but are not limited to, hematopoietic stem cells (HSC), stem cells of epithelial tissues, such as the skin and the lining of the gut, embryonic heart muscle cells, liver stem cells (see, e.g., WO 94/08598) and neural stem cells. See Stemple and Anderson, Cell, Vol. 71, pp. 973-985 (1992).

Epithelial stem cells (ESCs) or keratinocytes can be obtained from tissues, such as the skin and the lining of the gut by known procedures. See Rheinwald, Meth Cell Biol, Vol. 21A, p. 229 (1980). In stratified epithelial tissue, such as the skin, renewal occurs by mitosis of stem cells within the germinal layer, the layer closest to the basal lamina. Stem cells within the lining of the gut provide for a rapid renewal rate of this tissue. ESCs or keratinocytes obtained from the skin or lining of the gut of a patient or donor can be grown in tissue culture. See Pittelkow and Scott, Mayo Clinic Proc, Vol. 61, p. 771 (1986). If the ESCs are provided by a donor, a method for suppression of host versus graft reactivity, e.g., irradiation, drug or antibody administration to promote moderate immunosuppression, can also be used.

With respect to HSCs, any technique which provides for the isolation, propagation, and maintenance in vitro of HSCs can be used in this embodiment of the invention. Techniques by which this may be accomplished include:
(a) the isolation and establishment of HSC cultures from bone marrow cells isolated from the future host or a donor; or
(b) the use of previously established long-term HSC cultures, which may be allogeneic or xenogeneic.

Non-autologous HSC are used preferably in conjunction with a method of suppressing transplantation immune reactions of the future host/patient. In a particular embodiment of the present invention, human bone marrow cells can be obtained from the posterior iliac crest by needle aspiration. See, e.g., Kodo et al., J Clin Invest, Vol. 73, pp. 1377-1384 (1984). In a preferred embodiment of the present invention, the HSCs can be made highly enriched or in substantially pure form. This enrichment can be accomplished before, during, or after long-term culturing, and can be done by any techniques known in the art. Long-term cultures of bone marrow cells can be established and maintained by using, e.g., modified Dexter cell culture techniques [see Dexter et al., J. Cell Physiol., Vol. 91, p. 335 (1977)] or Witlock-Witte culture techniques. See Witlock and Witte, Proc Natl Acad Sci USA, Vol. 79, pp. 3608-3612 (1982).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

A further embodiment of the present invention relates to a method to treat, prevent or ameliorate a pathological condition associated with dysregulation of the ISP that comprises adminstering to a subject in need thereof an effective amount of a modulator of a protein selected from the group consisting of those disclosed in Table 13 or 25. In one embodiment, the modulator comprises one or more antibodies to said protein or fragments thereof, wherein said antibodies or fragments thereof can inhibit the biochemical function of said protein in said subject.

In another embodiment, the modulator comprises a peptide mimetic of a protein disclosed in Table 13 or 25. Suitable peptide mimetics to Table 13 or 25 proteins can be made according to conventional methods based on an understanding of the regions in a polypeptide required for protein activity. Briefly, a short amino acid sequence is identified in a protein by conventional structure function studies, such as deletion or mutation analysis of the wild-type protein. Once critical regions are identified, it is anticipated that if they correspond to a highly conserved portion of the protein that this region will be responsible for a critical function, such as protein-protein interaction. A small synthetic mimetic that is designed to look like said critical region would be predicted to compete with the intact protein and thus interfere with its function. The synthetic amino acid sequence could be composed of amino acids matching this region in whole or in part. Such amino acids could be replaced with other chemical structures resembling the original amino acids but imparting pharmacologically better properties, such as higher inhibitory activity, stability, half-life or bioavailability.

Also described herein are methods for the production of antibodies capable of specifically recognizing one or more differentially-expressed gene epitopes. Such antibodies may include, but are not limited to, polyclonal antibodies, monoclonal antibodies (mAbs), humanized or chimeric antibodies, single-chain antibodies, Fab fragments, F(ab′)₂ fragments, fragments produced by a Fab expression library, anti-idiotypic (anti-id) antibodies and epitope-binding fragments of any of the above. Such antibodies may be used, e.g., in the detection of a target protein in a biological sample, or alternatively, as a method for the inhibition of the biochemical function of the protein. Thus, such antibodies may be utilized as part of disease treatment methods, and/or may be used as part of diagnostic techniques whereby patients may be tested, e.g., for abnormal levels of polypeptides set forth in Table 13 or 25, or for the presence of abnormal forms of these polypeptides.

For the production of antibodies to the Table 13 or 25 polypeptides, various host animals may be immunized by injection with these polypeptides, or a portion thereof. Such host animals may include, but are not limited to, rabbits, mice, goats, chickens and rats, to name but a few. Various adjuvants may be used to increase the immunological response, depending on the host species including, but not limited to, Freund's (complete and incomplete); mineral gels, such as aluminum hydroxide; surface active substances, such as lysolecithin; pluronic polyols; polyanions; peptides; oil emulsions; keyhole limpet hemocyanin; dinitrophenol; and potentially useful human adjuvants, such as Bacille Calmette-Guerin (BCG) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals, such as those described above, may be immunized by injection with a Table 13 or 25 polypeptide, or a portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique [see Kohler and Milstein, Nature, Vol. 256, pp. 495-497 (1975); and U.S. Pat. No. 4,376,110], the human B-cell hybridoma technique [Kosbor et al., Immunol Today, Vol. 4, p. 72 (1983); and Cole et al., Proc Natl Acad Sci USA, Vol. 80, pp. 2026-2030 (1983)], and the EBV-hybridoma technique. See Cole et al., Monoclonal Antibodies and Cancer Therapy, Alan R. Liss, Inc., pp. 77-96 (1985). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies”[see Morrison et al., Proc. Natl. Acad. Sci. USA, Vol. 81, pp. 6851-6855 (1984); Neuberger et al., Nature, Vol. 312, pp. 604-608 (1984); Takeda et al., Nature, Vol. 314, pp. 452-454 (1985)] by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single-chain antibodies [see U.S. Pat. No. 4,946,778; Bird, Science, Vol. 242, pp. 423-426 (1988); Huston et al., 1988, Proc Natl Acad Sci USA, Vol. 85, pp. 5879-5883 (1988); and Ward et al., Nature, Vol. 334, pp. 544-546 (1989)] can be adapted to produce differentially-expressed gene single-chain antibodies. Single-chain antibodies are formed by linking the heavy- and light-chain fragments of the Fv region via an amino acid bridge, resulting in a single-chain polypeptide.

Most preferably, techniques useful for the production of “humanized antibodies” can be adapted to produce antibodies to the polypeptides, fragments, derivatives and functional equivalents disclosed herein. Such techniques are disclosed in U.S. Pat. Nos. 5,932,448, 5,693,762, 5,693,761, 5,585,089, 5,530,101, 5,910,771, 5,569,825, 5,625,126, 5,633,425, 5,789,650, 5,545,580, 5,661,016 and 5,770,429, the disclosures of all of which are incorporated by reference herein in their entirety.

Antibody fragments that recognize specific epitopes may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed [see Huse et al., Science, Vol. 246, pp. 1275-1281 (1989)] to allow rapid and easy identification of monoclonal Fab fragments with the desired specificity.

As contemplated herein, an antibody of the present invention can be preferably used in a diagnostic kit for detecting levels of a protein disclosed in Table 13 or 25 in a biological sample, as well as in a method to diagnose subjects suffering from pathological conditions associated with dysregulation of the ISP who may be suitable candidates for treatment with modulators to a protein selected from the group consisting of those disclosed in Table 13 or 25. Preferably, said detecting step comprises contacting said appropriate tissue cell, e.g., biological sample, with an antibody which specifically binds to a Table 13 or 25 polypeptide or a fragment thereof and detecting specific binding of said antibody with a polypeptide in said appropriate tissue, cell or sample wherein detection of specific binding to a polypeptide indicates the presence of a polypeptide set forth in Table 13 or 25 or a fragment thereof.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex. At this point, a second antibody, labeled with a reporter molecule capable of inducing a detectable signal, is then added and incubated, allowing time sufficient for the formation of a ternary complex of antibody-antigen-labeled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for a Table 13 or 25 polypeptide or a fragment thereof.

The most commonly used reporter molecules in this type of assay are either enzymes, fluorophore- or radionuclide-containing molecules. In the case of an enzyme immunoassay, an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta (β)-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of Table 13 or 25 polypeptide which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well-established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The pharmaceutical compositions of the present invention may also comprise substances that inhibit the expression of a protein disclosed in Table 13 or 25 at the nucleic acid level. Such molecules include ribozymes, antisense oligonucleotides, triple-helix DNA, RNA aptamers, siRNA and/or single- or double-stranded RNA directed to ad appropriate nucleotide sequence of nucleic acid encoding such a protein. These inhibitory molecules may be created using conventional techniques by one of skill in the art without undue burden or experimentation. For example, modifications, e.g., inhibition, of gene expression can be obtained by designing antisense molecules, DNA or RNA, to the control regions of the genes encoding the polypeptides discussed herein, i.e., to promoters, enhancers and introns. For example, oligonucleotides derived from the transcription initiation site, e.g., between positions −10 and +10 from the start site may be used. Notwithstanding, all regions of the gene may be used to design an antisense molecule in order to create those which gives strongest hybridization to the mRNA and such suitable antisense oligonucleotides may be produced and identified by standard assay procedures familiar to one of skill in the art.

Similarly, inhibition of gene expression may be achieved using “triple-helix” base-pairing methodology. Triple-helix pairing is useful because it causes inhibition of the ability of the double helix to open sufficiently for the binding of polymerases, transcription factors or regulatory molecules. Recent therapeutic advances using triplex DNA have been described in the literature. See Gee et al., Huber and Carr, eds., Molecular and Immunologic Approaches, Futura Publishing Co., Mt. Kisco, N.Y. (1994). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules. See Grassi and Marini, Ann Med, Vol. 28, pp. 499-510 (1996); and Gibson, Cancer Metast Rev, Vol. 15, pp. 287-299 (1996). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. See Cotten et al., EMBO J, Vol. 8, pp. 3861-3866 (1989). In particular, a ribozyme coding DNA sequence, designed according to conventional, well-known rules and synthesized, e.g., by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter, e.g., a glucocorticoid or a tetracycline response element, is also introduced into this construct so that ribozyme expression can be selectively controlled. For saturating use, a highly and constituently active promoter can be used. tDNA genes, i.e., genes encoding tRNAs, are useful in this application because of their small size, high rate of transcription and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly, the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA [see Good et al., Gene Ther, Vol. 4, pp. 45-54 (1997)] that can specifically inhibit their translation.

Gene specific inhibition of gene expression may also be achieved using conventional single- or double-stranded RNA technologies. A description of such technology may be found in WO 99/32619 which is hereby incorporated by reference in its entirety. In addition, siRNA technology has also proven useful as a means to inhibit gene expression. See Cullen, Br Nat Immunol, Vol. 3, No. 7, pp. 597-599 (2002); and Martinez et al., Cell, Vol. 110, No. 5, p. 563 (2002).

Antisense molecules, triple-helix DNA, RNA aptamers, dsRNA, ssRNA, siRNA and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters, such as T7 or SP6. Alternatively, cDNA constructs that synthesize antisense RNA constitutively or inducibly can be introduced into cell lines, cells or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well-known in the art.

Detection of mRNA levels of proteins disclosed herein may comprise contacting a biological sample or even contacting an isolated RNA or DNA molecule derived from a biological sample with an isolated nucleotide sequence of at least about 20 nucleotides in length that hybridizes under high-stringency conditions, e.g., 0.1×SSPE or SSC, 0.1% SDS, 65° C.) with the isolated nucleotide sequence encoding a polypeptide set forth in Table 13 or 25. Hybridization conditions may be highly-stringent or less-highly stringent. In instances wherein the nucleic acid molecules are deoxyoligonucleotides (“oligos”), highly-stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). Suitable ranges of such stringency conditions for nucleic acids of varying compositions are described in Krause and Aaronson, Methods Enzymol, Vol. 200, pp. 546-556 (1991), in addition to Maniatis et al., cited above.

In some cases, detection of a mutated form of the gene which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acids, in particular mRNA, for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled nucleotide sequences encoding a polypeptide encoded by a gene disclosed in Table 13 or 25. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science, Vol. 230, p. 1242 (1985). Sequence changes at specific locations may also be revealed by nuclease protection assays, such as RNase and S1 protection or the chemical cleavage method. See Cotton et al., Proc Natl Acad Sci USA, Vol. 85, pp. 4397-4401 (1985). In addition, an array of oligonucleotides probes comprising nucleotide sequence encoding the Table 13 or 25 polypeptides or fragments of such nucleotide sequences can be constructed to conduct efficient screening of, e.g., genetic mutations. Array technology methods are well-known and have general applicability and can be used to address a variety of questions in molecular genetics including gene expression, genetic linkage and genetic variability. See, e.g., Chee et al., Science, Vol. 274, pp. 610-613 (1996).

The diagnostic assays offer a process for diagnosing or determining a susceptibility to disease through detection of mutation in the gene of a polypeptide set forth in Table 13 or 25 by the methods described. In addition, such diseases may be diagnosed by methods comprising determining from a sample derived from a subject an abnormally decreased or increased level of polypeptide or mRNA. Decreased or increased expression can be measured at the RNA level using any of the methods well-known in the art for the quantitation of polynucleotides, such as, e.g., nucleic acid amplification, for instance, PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagnostic kit for detecting mRNA levels (or protein levels) which comprises:
(a) a polynucleotide of a polypeptide set forth in Table 13 or 25 or a fragment thereof;
(b) a nucleotide sequence complementary to that of (a);
(c) a polypeptide of Table 13 or 25 of the present invention encoded by the polynucleotide of (a),
(d) an antibody to the polypeptide of (c);
(e) an RNAi sequence complementary to that of (a); and
(f) a peptide mimetic to a Table 13 or 25 protein.

It will be appreciated that in any such kit, (a), (b), (c), (d), (e) or (f) may comprise a substantial component. Such a kit will be of use in diagnosing a disease or susceptibility to a disease, particularly to a disease or condition associated with dysregulation of the ISP, e.g., Type II diabetes or the Type A syndrome of insulin resistance.

The nucleotide sequences of the present invention are also valuable for chromosome localization. The sequence is specifically targeted to, and can hybridize with, a particular location on an individual human chromosome. The mapping of relevant sequences to chromosomes is an important first step in correlating those sequences with gene associated disease. Once a sequence has been mapped to a precise chromosomal location, the physical position of the sequence on the chromosome can be correlated with genetic map data. Such data are found in, e.g., McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

An additional embodiment of the invention relates to the administration of a pharmaceutical composition, in conjunction with a pharmaceutically acceptable carrier, excipient or diluent, for any of the therapeutic effects discussed above. Such pharmaceutical compositions may comprise, for example, a polypeptide set forth in Table 13 or 25, antibodies to that polypeptide, mimetics, agonists, antagonists, inhibitors or other modulators of function of a Table 13 or 25 polypeptide or gene therefore. The compositions may be administered alone or in combination with at least one other agent, such as stabilizing compound, which may be administered in any sterile, biocompatible pharmaceutical carrier, including, but not limited to, saline, buffered saline, dextrose and water. The compositions may be administered to a patient alone, or in combination with other agents, drugs or hormones.

In addition, any of the therapeutic proteins, antagonists, antibodies, agonists, antisense sequences or other modulators described above may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment, prevention or amelioration of pathological conditions associated with abnormalities in the ISP. Using this approach, one may be able to achieve therapeutic efficacy with lower dosages of each agent, thus reducing the potential for adverse side effects. Antagonists, agonists and other modulators of the human polypeptides set forth in Table 13 or 25, and genes encoding said polypeptides may be made using methods which are generally known in the art.

The pharmaceutical compositions encompassed by the invention may be administered by any number of routes including, but not limited to, oral, intravenous, intramuscular, intraarticular, intraarterial, intramedullary, intrathecal, intraventricular, transdermal, subcutaneous, intraperitoneal, intranasal, enteral, topical, sublingual or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol or sorbitol; starch from corn, wheat, rice, potato or other plants; cellulose, such as methyl cellulose, hydroxypropylmethyl-cellulose, or sodium carboxymethylcellulose; gums including arabic and tragacanth; and proteins, such as gelatin and collagen. If desired, disintegrating or solubilizing agents may be added, such as the cross-linked polyvinyl pyrrolidone, agar, alginic acid or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid or liquid polyethylene glycol with or without stabilizers.

Pharmaceutical formulations suitable for parenteral administration may be formulated in aqueous solutions, preferably in physiologically compatible buffers, such as Hanks' solution, Ringer's solution or physiologically buffered saline. Aqueous injection suspensions may contain substances that increase the viscosity of the suspension, such as sodium carboxymethyl cellulose, sorbitol or dextran. Additionally, suspensions of the active compounds may be prepared as appropriate oily injection suspensions. Suitable lipophilic solvents or vehicles include fatty oils, such as sesame oil; or synthetic fatty acid esters, such as ethyl oleate or triglycerides or liposomes. Non-lipid polycationic amino polymers may also be used for delivery. Optionally, the suspension may also contain suitable stabilizers or agents which increase the solubility of the compounds to allow for the preparation of highly-concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with many acids including, but not limited to, hydrochloric, sulfuric, acetic, lactic, tartaric, malic, succinic, etc. Salts tend to be more soluble in aqueous or other protonic solvents than are the corresponding free base forms. In other cases, the preferred preparation may be a lyophilized powder that may contain any or all of the following: 1-50 mM histidine, 0.1-2% sucrose, and 2-7% mannitol, at a pH range of 4.5-5.5, that is combined with buffer prior to use.

After pharmaceutical compositions have been prepared, they can be placed in an appropriate container and labeled for treatment of an indicated condition. Such labeling would include amount, frequency and method of administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs or pigs. The animal model may also be used to determine the appropriate concentration range and route of administration. Such information can then be used to determine useful doses and routes for administration in humans.

A therapeutically-effective dose refers to that amount of active ingredient which ameliorates the symptoms or condition. Therapeutic efficacy and toxicity may be determined by standard pharmaceutical procedures in cell cultures or experimental animals, e.g., the dose therapeutically effective in 50% of the population (ED₅₀) and the dose lethal to 50% of the population (LD₅₀). The dose ratio between toxic and therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities and tolerance/response to therapy. Long-acting pharmaceutical compositions may be administered every 3-4 days, every week or once every two weeks depending on half-life and clearance rate of the particular formulation.

Normal dosage amounts may vary from 0.1-100,000 mg, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114, 5,505,962, 5,641,515, 5,681,811, 5,700,486, 5,766,633, 5,792,451, 5,853,748, 5,972,387, 5,976,569 and 6,051,561.

The following Examples illustrate the present invention, without in any way limiting the scope thereof.

### EXAMPLES

The following methods are employed to perform the examples provided below:

Drosophila Genetics

Flies are kept on standard corn meal food. All crosses are done at 25° C. The genetic background for all the flies used is w1118. UAS-Dp110^{D954A} and UAS-Dp110^{CAAX} are used to overexpress a dominant negative form and a constitutively active form of the fly PI3K catalytic subunit, respectively. See Leevers et al. (1996), supra. UAS-dPTEN is used to over-express fly PTEN. See Huang et al., Development, Vol. 126, pp. 5365-5372 (1999). UAS-dAkt1 is used to over-express fly Akt. See Verdu et al., Nat. Cell Biol., Vol. 1, pp. 500-506 (1999). UAS-dfoxo is generated using a cDNA of the gene CG3143 (see Li, unpublished data) and used to overexpress fly Foxo, a putative forkhead-domain transcription factor and a homolg of human Foxo family transcription factor.

Transgene Expression and Total RNA Isolation

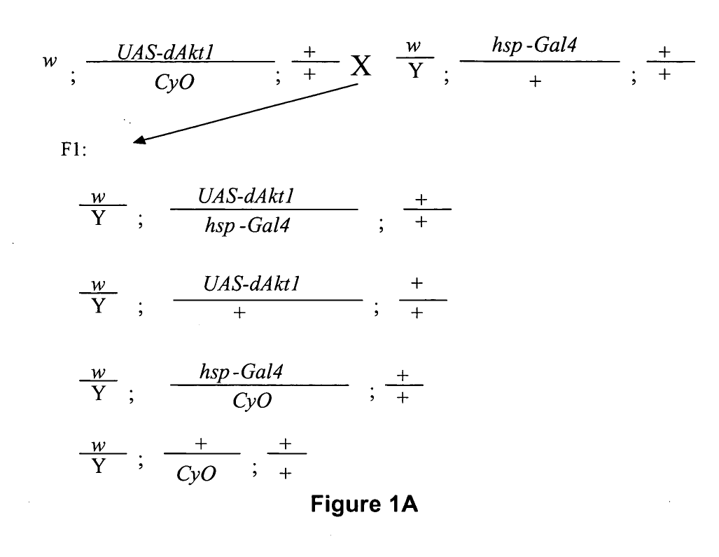

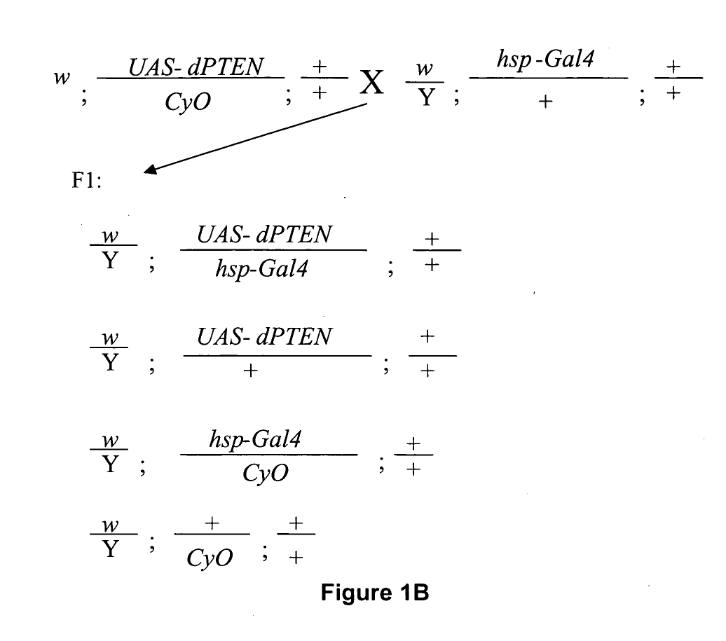

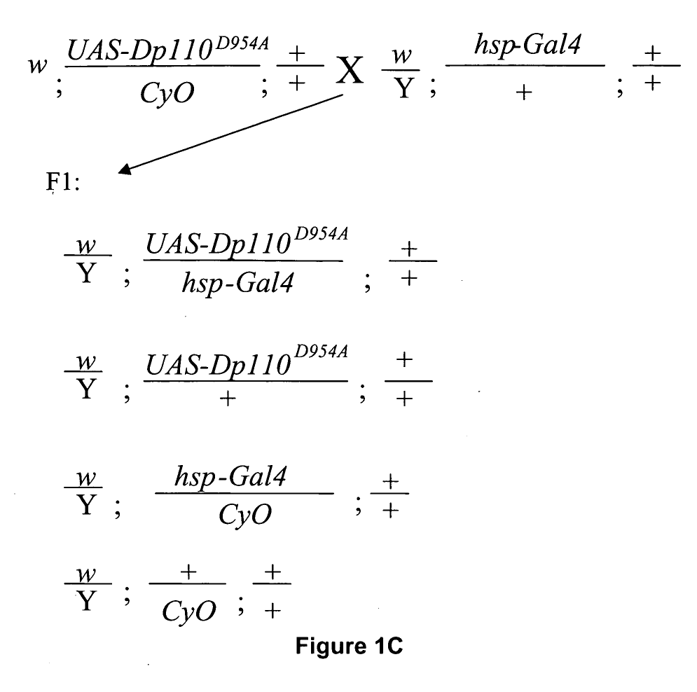

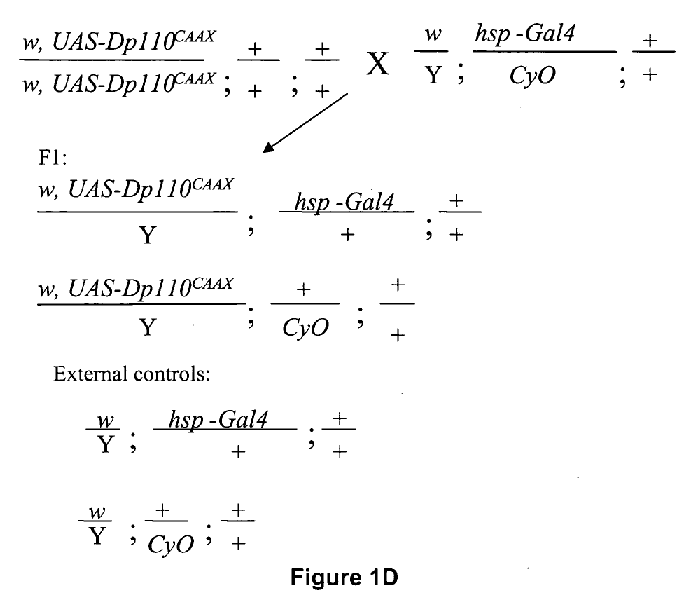

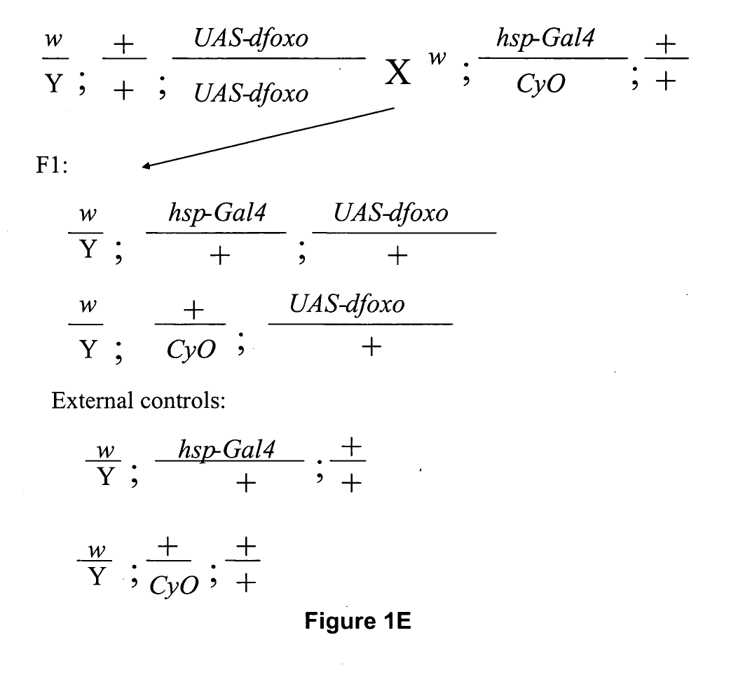

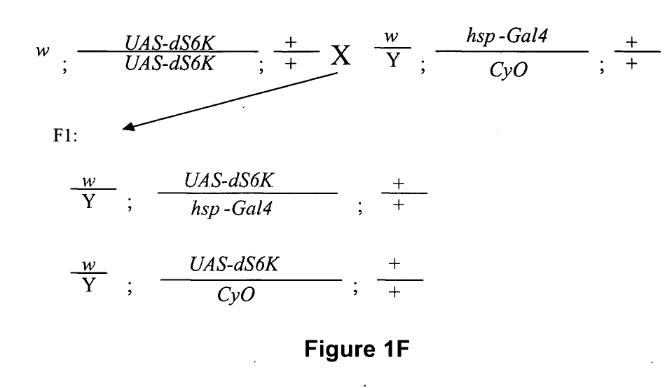

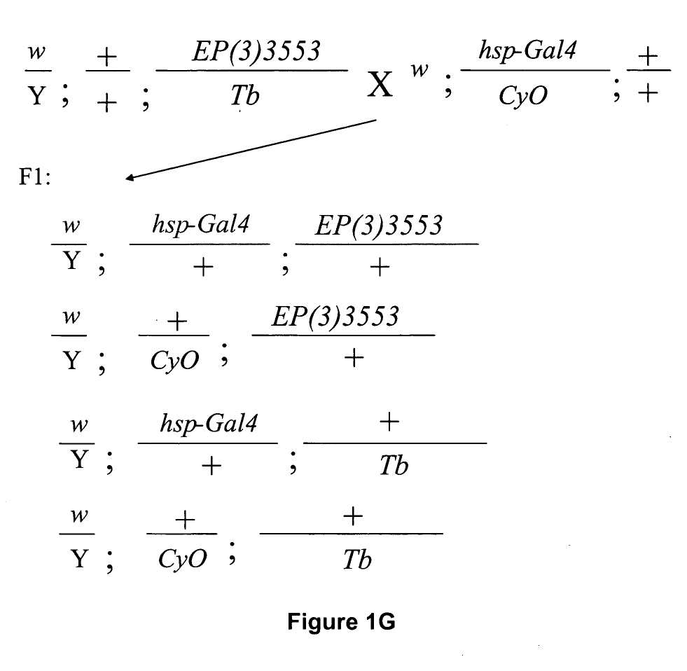

Based on the GAL4/UAS system [see Brand and Perrimon, Development, Vol. 118, pp. 401-415 (1993)] hsp-Gal4 is used to induce the overexpression of the UAS-transgenes (UAS-Dp110^{D954A}, UAS-Dp110^{CAAX}, UAS-dPTEN, UAS-dAkt1 and UAS-dfoxo). The genetic crosses are shown in FIG. 1.

Heat shock-dependent induction is done only in adult male flies to avoid sampling RNA from ovaries. For dAkt1, dPTEN and Dp110^{D954A} overexpression experiments, the genetic cross (see FIGS. 1A, 1B and 1C) generate male progenies of 4 genotypes: flies carrying both hsp-Gal4 and UAS-transgene (hsp-Gal4/UAS), flies carrying only hsp-Gal4 (hsp-Gal4), flies carrying only UAS-transgene (UAS), and flies carrying neither hsp-Gal4 nor UAS-transgene (CyO). For Dp110CAAX and dfoxo overexpression experiments (see FIGS. 1D and 1E for genetic crosses), external control groups of genotypes hsp-Gal4/+ and +/CyO are used. In each of the 5 overexpression experiments, levels of 3 factors are involved: +/− heat shock, +/− hsp-Gal4 and +/− UAS-transgene. Thus, there are 8 combinations of genotype and treatment. For each combination, 4 independent total RNA samples, each from ˜50 7- to 10-day-old adult male files, are made. This resulted in 32 RNA samples per experiment. Non-heat-shock treated flies are kept at 25° C. The heat-shock treated flies are kept at 37° C. for 1 hour, followed by a recovery period of 3 hours at 25° C. This scheme is repeated 6 times before RNA isolation. The total RNA is extracted using Trizol reagent (GIBCO/BRL) and then further purified using RNeasy columns (Qiagen) following the RNA cleanup protocol.

Microarray Experiments

Microarray experiments are performed using Affymetrix (Santa Clara, Calif.) Drosophila GeneChip™ and following the methods described in the Affymetrix GeneChip™ Expression Technical Manual. Briefly, 10 μg of total RNA per sample is used to synthesize double-stranded cDNA. The cDNA is then transcribed in vitro to form biotin-labeled cRNA using Enzo BioArray® High Yield RNA transcript labeling kit (Enzo Biochem). Fifteen (15) μg of fragmented cRNA is hybridized to each array. Hybridization, washing, staining and scanning of the target cRNA to the arrays are performed as per the Affymetrix GeneChip™ manual. For each overexpression experiment, 32 arrays are used with one array for one RNA sample.

Data Analysis

The GeneChip™ Drosophila genome array used in this study contains 13,966 gene sequences predicted from the annotation of the Drosophila genome Release 1.0. See Adams, et al., Science, Vol. 287, pp. 2185-2195 (2000).

Each sequence is represented on the array by a set of 14 pairs of perfect match (PM) and mismatch (MM) oligonucleotide probes (25 mers). Data are collected at the level of the transcript (referred to herein by gene name). The hybridization intensity data are calculated from the images generated by the Gene Array scanner (Affymetrix), using the Affymetrix Microarray Suite (MAS) 4.0. The average difference (Avg Diff) between the PM signal and the MM signal for every probe set is calculated, and the mean Avg Diff for each array is set to 2,500 by linearly scaling array values. Next, the negative Avg Diff values on the array that could interfere with subsequent analysis are truncated and every Avg Diff value below 20 is assigned an Avg Diff of 20. For each experiment, all the arrays are then re-scaled to 5,000,000 of total target intensity. An unpaired t-test for each individual gene is carried out for the following pairwise comparisons for each experiment:
(1) Heat-shocked hsp-Gal4/UAS-transgene flies versus heat-shocked hsp-Gal4 flies (to eliminate effects caused by heat shock and the overexpression of Gal4 protein only);
(2) Heat-shocked hsp-Gal4/UAS-transgene flies versus heat-shocked UAS-transgene flies (to eliminate effects caused by UAS-transgene only);
(3) Heat-shocked hsp-Gal4/UAS-transgene flies versus heat-shocked CyO flies (to eliminate a small number of genes, for which the heat-shocked no hsp-Gal4 and no UAS-transgene group gave high background induction); and
(4) Heat-shocked hsp-Gal4/UAS-transgene flies versus non-heat-shocked hsp-Gal4 plus non-heat-shocked UAS-transgene plus non-heat-shocked CyO flies (to eliminate a small number of heat-shock responsive genes).

The differentially-expressed genes (DEGs) by the overexpression of each of these transgenes are identified based on the following criteria: for each of the above t-tests, genes that had significant 2-fold or above changes in mean Avg Diff (p≦0.005) are selected. Additionally, the higher mean Avg Diff of a pairwise comparison for a given gene is above or equal to 200. Since heat-shocked hsp-Gal4 fly samples gave most of the non-specific effects, the fold changes calculated from the first t-test comparison, i.e., heat-shocked hsp-Gal4/UAS-transgene flies versus heat-shocked hsp-Gal4 flies, are used to represent the most conservative estimate of the fold change between experimental and control groups for each gene.

Gene Ontology Analysis

Every probe set on the GeneChip™ Drosophila genome array is annotated by integrating the information on the gene ontology (GO) web site (http://www.geneontology.org) with the information available from FlyBase (http://www.fruitfly.org). We first associate each probe set on the chip with its current gene entry in FlyBase. Next, we associate each gene to its available GO annotations for molecular function and biological process. We exclude annotations derived exclusively from electronic annotation (evidence code IEA). We restore the GO tree structure and determine the number of genes that are annotated as belonging to a particular GO term and its child GO terms.

Quantitative Real-Time PCR (QRT-PCR)

To confirm the microarray data analysis results, QRT-PCR is carried out on selected genes. Reverse transcription step is done using TaqMan Reverse Transcription Reagents (Roche Molecular Systems, Inc., Pleasanton, Calif.). QRT-PCR is performed with the reverse transcription product, SYBR® Green PCR Master Mix (Applied Biosystems, Foster City, Calif.), and gene-specific primers (Table 2), using ABI Prism® 7900HT Sequence Detection System. RNA levels of the rp49 gene, which encodes a ribosomal protein, are used as internal normalization controls.

### Example 1

Genome Wide-Expression Analysis

In order to identify genes whose transcription is effected by activation of the ISP in Drosophila, a genome wide-expression analysis is performed. To this end, several proteins known to be involved in the ISP are overexpressed using the Gal4/UAS system [see Brand and Perrimon (1993), supra], specifically, dAkt1, Dp110 and dPTEN are chosen because they have been shown to be critical components of this pathway and are conserved between humans and Drosophila in both sequence homology and function. dfoxo was also included because it has been demonstrated that the forkhead-domain transcription factor is downstream of and regulated by Akt in humans and C. elegans. See Kops et al., Nature, Vol. 398, pp. 630-634 (1999); Brunet et al., Cell, Vol. 96, pp. 857-868 (1999); Guo et al., J Biol Chem, Vol. 274, pp. 17184-17192 (1999); Rena et al., J Biol Chem, Vol. 274, pp. 17179-17183 (1999); and Ogg et al., Nature, Vol. 389, pp. 994-999 (1997).

These genes are over-expressed in adult flies to increase the chance of identifying direct downstream targets of ISP and to avoid identifying secondary-effect genes due to developmental effects on gene expression. The heat shock scheme used to induce the transgenes is long enough to achieve a high level of UAS-transgene expression based on our unpublished kinetics of transgene overexpression, but short enough so that genes identified represent more direct downstream targets of ISP and less likely secondary effects of gene expression.

Following overexpression of these genes, transcript profiles are analyzed using Affymetrix Drosophila GeneChip™ and the differentially expressed genes for each overexpressed transgene are identified as described above. Genes are represented on the chip by one or more probe sets, which correspond to approximately 13,300 unique genes according to Release 1.0 of the Drosophila genome. See Adams et al. (2000), supra. Gene expression is verified by QRT-PCR with gene-specific primers (see Example 3 below).

### Example 2

Expression Patterns of DEGs

The expression patterns of all the DEGs that passed our filtering criteria (see methods above) in each overexpression experiment are organized by hierarchical clustering. See Eisen et al., Proc Natl Acad Sci USA, Vol. 95, No. 25, pp. 14863-14868 (1998). A total of 128, 339, 85, 16 and 234 genes are found to be differentially-regulated following dAkt1, Dp110CAAX, dPTEN, Dp110^{D954A} and dfoxo over-expression, respectively (see Table 3), at a significance level of p≦0.005. These correspond to approximately 0.96%, 2.5%, 0.64%, 0.12% and 1.75% of the genes represented on the array, respectively. The top 20 down-regulated and top 20 up-regulated genes in response to each UAS-transgene overexpression (except for UAS-Dp110^{D954A} overexpression, where only 16 DEGs are identified) are shown in Tables 5-8.

Our approach identifies less DEGs following the overexpression of negative regulators of the ISP, Dp110^{D954A} and dPTEN, than positive regulators dAkt1 and Dp110^{CAAX}. One possible explanation could be that insulin signaling in adult flies are maintained at a relatively lower level than that of earlier developmental stages and thus the effects of up-regulation of the insulin signal may be easier to observe than down-regulation of the insulin signal. Overexpression of Dp110^{D954A} gives a much lower percentage of DEGs. This could be because the dominant negative form of Dp110 induced in our experiment is not a strong effector.

All the DEGs and all the over-expression experiments are hierarchically clustered to see which experiments are more similar to each other in terms of fold change as compared to others. It is interesting to see that overexpression of dAkt1 and Dp110CAAX, two positive regulators of insulin signaling, are clustered together, whereas overexpression of dPTEN and Dp110^{D954A}, two negative regulators of insulin signaling, are clustered together. Furthermore, DEGs whose expression levels are high in dAkt1 and Dp110^{CAAX} experiments generally have low-expression levels in dPTEN and Dp110^{D954A} experiments and vice versa. Taken together, the above observations demonstrate that the transcription of these DEGs are regulated by the ISP.

Since dfoxo activity is down-regulated through phosphorylation by dAkt1 in mammals and C. elegans, its activity is likely down-regulated in Drosophila as well. Thus, over-expressing dfoxo in a subject could potentially antagonize insulin signal transduction through the dAkt-dfoxo branch. Consistent with this idea, we find that over-expression effect of dfoxo on global patten of gene expressing is more similar to those caused by dPTEN and Dp110^{D954A}.

### Example 3

Verification of Microarray Expression Data with QRT-PCR

To confirm the differential-expression results described above and to identify best gene markers to complement genetic studies, e.g., validation of genetic screen hits, QRT-PCR is carried out on selected candidate genes. A total of 50 genes (including Zw, diptericin, diptericin B, see below) from the following categories are selected:
(1) Genes up- or down-regulated by dAkt1, Dp110CAAX, dfoxo, dPTEN or Dp110^{D954A} overexpression alone, respectively;
(2) Genes up- or down-regulated by both dAkt1 and Dp110^{CAAX} overexpression; and
(3) Genes up- or down-regulated by both dPTEN and Dp110^{D954A} overexpression.

In all, 66 QRT-PCR reactions for the 50 DEGs are conducted and only 2 failed to confirm the microarray results because of failing to pass the t-test p-value filter (data not shown). These results show that the changes in the relative expression level, as measured by QRT-PCR, are generally consistent with the data obtained with the oligonucleotide arrays.

### Example 4

Functional Classification of Differentially-Expressed Transcripts

Molecular genetic studies with Drosophila have demonstrated that the ISP is involved in the regulation of growth, cell proliferation, metabolism and aging. In order to find out what biological processes and functional products encoded by the genes showing differential transcription responses are affected by the over-expression of ISP genes in Drosophila, we use the annotation project directed by the GO Consortium (http://www.geneontology.org) to functionally classify these differentially-regulated genes. The objective of GO is to provide controlled vocabularies for the description of the molecular function, biological process and cellular component of gene products. See Ashburner et al., Nat Genet, Vol. 25, pp. 25-29 (2000). The GO contains information on approximately 46% (6,423/13,966) of the probe sets present on the arrays employed herein.

Detailed biological process and molecular function classifications of the genes differentially-regulated by the over-expression of the ISP components performed herein are presented in Tables 10 and 11, respectively. Primary GO terms under the 2 major GO terms “biological process” and “molecular function” and selected secondary GO terms showing more detailed annotation of the primary terms are shown. For secondary GO terms, only those containing DEGs from at least 3 out of the 5 over-expression experiments are shown.

As shown in Table 3, a total of 128, 339, 85, 16 and 234 genes are found to be differentially-regulated following dAkt1, Dp110^{CAAX}, dPTEN, Dp110^{D954A} and dfoxo overexpression, respectively, at a significance level of p≦0.005. These correspond to approximately 0.96%, 2.5%, 0.64%, 0.12% and 1.75% of the genes represented on the array, respectively. If the DEGs from each overexpression experiment comprise approximately 0.96%, 2.5%, 0.64%, 0.12% and 1.75% of the genes represented on the array, respectively, then for each of the above overexpression experiments, if there is no relationship between the differential expression and molecular functions or biological processes, by random chance we expect (for each GO term) 0.96/100, 2.5/100, 0.64/100, 0.12/100 and 1.75/100 as many DEGs in each overexpression experiment as there are on the entire chip, respectively. However, Tables 10 and 11 show that, for several GO terms, the percentages of DEGs relative to the total number of genes in that GO category represented on the chip is much higher than the percentage of DEGs from each over-expression experiment relative to the total number of genes represented on the array. Therefore, the biological processes defined by these GO terms are likely regulated by the ISP components.

Interestingly, one of the major biological processes that may be regulated by the insuling signaling pathway is the “defense/immune response” (see Table 9). This is also confirmed by molecular function classification shown in Table 10 (GO term “defense/immunity proteins”). The DEGs under the biological process GO term “defense response” from different overexpression experiments are shown in Table 11. This discovery is of great interest because insulin signaling has not previously been shown to directly regulate Drosophila innate immunity.

Innate immunity is the first-line defense of multicellular organisms against pathogenic challenges. Invertebrates and vertebrates share a common ancestry for this defense system, illustrated by the striking conservation of the intracellular signaling pathways that regulate the rapid transcriptional response to infection in Drosophila and mammals. See Hoffmann et al., Science, Vol. 284, pp. 1313-1318 (1999); and Borregaard et al., Immunol Today, Vol. 21, pp. 68-70 (2000).

It has been shown that transcriptional induction of innate immune response is controlled by at least 2 distinct NFκB signaling pathways, Toll and Imd. See Imler and Hoffmann, Curr Opin Microbiol, Vol. 3, pp. 16-22 (2000). In addition to these 2 pathways, the JNK and JAK/STAT pathways have also been shown to contribute to the expression of microbial challenge-induced genes. See Boutros et al., Devel Cell, Vol. 3, pp. 711-722 (2002).

Recently, several studies have investigated the transcriptional responses to microbial infection in Drosophila using DNA microarrays [see De Gregorio, Proc Natl Acad Sci USA, Vol. 98, pp. 12590-12595 (2001); Irving, Proc Natl Acad Sci USA, Vol. 98, pp. 15119-15124 (2001); and Boutros et al. (2002), supra] and target genes involved in immune responses were identified. By comparing DEGs shown in Table 11 to immune responsive genes identified in these microarray studies, we show that overexpression of ISP components affect genes in both Toll and Imd pathways. For the Toll pathway, Toll receptor ligand, spatzle, the downstream protein kinase, pelle and Toll pathway targets, such as drosomycin, Mtk, IM1 and IM2 are affected. For the Imd pathway, the Imd pathway targets, such as diptericin and diptericin B are affected. Also, CG8193 (see Table 11) encodes a monophenol monooxygenase that plays a role in melanization, which is a common defense mechanism among invertebrates and involved in both pigmentation and wound healing. See De Gregorio et al. (2001), supra. The genes shown in Table 11 represent only well-curated genes by GO.

To further estimate the percentages of DEGs from each overexpression experiment that are involved in immune responses, the 400 Drosophila immune-regulated genes [referred to as DIRGs in De Gregorio et al. (2001), supra] from the dataset of De Gregorio et al. (2001), supra, are selected and overlaps between DEGs from each of the different overexpression experiments and these genes identified as DIRGs are analyzed. Results indicate that approximately 22%, 12%, 15% and 13% of the DEGs from dAkt1, Dp110CAAX, dfoxo and dPTEN overexpression experiments, respectively, are Drosophila immune-regulated genes. One of the molecular function classes “serine-type peptidase” also show high relative numbers of DEGs (see Table 10). Since trypsin-like serine proteases and their inhibitors, serpins, play a central role in the insect immune response [see Levashina et al., Science, Vol. 285, pp. 1917-1919 (1999)], we look at the DEGs in this GO class, as well as the DEGs in the molecular function GO term “enzyme inhibitor” (see Table 10). Surprisingly, seven trypsin-like serine-protease-encoding DEGs (CG12385, CG12351, CG8871, CG16749, CG6467, CG9645 and CG11842) in the GO term “serine-type peptidase” and three serpin-encoding DEGs (CG3801, CG6687 and CG18525) in the GO term “enzyme inhibitor” from different insulin signalling pathway gene over-expression experiments overlap with the DIRGs, mentioned above.

The above evidence indicates that the ISP likely crosstalks with NFκB signaling pathways and plays an important role in regulating defense/immune responses in Drosophila. Previous studies in mammalian systems support this discovery. It has been shown that tumor necrosis factor ax (TNFα) and platelet-derived growth factor (PDGF) induced NFκB activation requires Akt and it has been indicated that Akt is part of a signalling pathway that is necessary for inducing key immune and inflammatory responses. See Ozes et al., Nature, Vol. 401, pp. 82-85 (1999); Romashkova and Makarov, Nature, Vol. 401, pp. 86-90 (1999); Madrid et al., Mol Cell Biol, Vol. 20, pp. 1626-1638 (2000); and Burow et al., Biochem Biophys Res Commun, Vol. 271, pp. 342-345 (2000).

Briefly, TNFα or PDGF activates PI3K and its downstream target Akt, which activates IκB kinase (IKK). The activation of NFκB involves phosphorylation of IκB by IKK, and subsequent IκB ubiquitination and degradation. Therefore, in Drosophila, it is possible that the ISP contributes to the regulation of innate immunity through activating NFκB transcription factors, such as Rel and Dif. Previous studies in mammalian systems also demonstrated an important role of PI3K in regulating innate immune responses, such as phagocytosis. Taken together, our results and previous studies in other systems support a role for the ISP in regulating innate immunity in Drosophila via interacting with other signaling pathways, such as NFκB pathways.

Many metabolic processes show high relative numbers of DEGs from multiple over-expression experiments (see Table 9), consistent with the findings that the ISP regulates metabolism in Drosophila. See Bohni et al., Cell, Vol. 97, pp. 865-875 (1999); Britton et al., Dev Cell, Vol. 2, pp. 239-249 (2002); and Brogiolo et al., Curr Biol, Vol. 11, pp. 213-221 (2001).

It is significant to see that, Zwischenferment (Zw), a gene encoding a rate-limiting enzyme (glucose-6-phosphate 1-dehydrogenase) in the pentose-phosphate shunt (PPP), is regulated by ISP (see Tables 9 and 10). The major role of PPP pathway is to generate NADPH-reducing power needed for fatty acid synthesis. It has been shown that Zw gene expression was up-regulated in sugar-fed but not in starved Drosophila larvae. See Zinke et al., EMBO J, Vol. 21, pp. 6162-6173 (2002). We see that Zw is up-regulated by dAkt1 and Dp110^{CAAX}, but down-regulated by dfoxo, which is a good indication that insulin signaling up-regulates PPP pathway possibly through Dp110/dAkt1/dfoxo branch, and consistent with the observation that insulin promotes fatty acid synthesis. Another gene, CG6283, encoding a triacylglycerol lipase, has been shown to be down-regulated in sugar-fed but not in starved Drosophila larvae. See Zinke et al. (2002), supra. Our resultd show that this gene is down-regulated by Dp110^{CAAX}, but up-regulated by dfoxo. This is also consistent with the fact that insulin inhibits fatty acid breakdown. Several glucose/sugar transporters are also differentially regulated by the over-expression of ISP components (see Table 10), indicating that insulin signaling also regulate carbohydrate/glucose metabolism in Drosophila. To further dissect which metabolic pathways are affected by insulin signalling pathway component over-expression, we also use annotation information of metabolic pathways from Kyoto Encyclopedia of Genes and Genomes (KEGG), http://www.genome.ad.jp/kegg/. A detailed metabolic pathway classification of the genes differentially regulated by the overexpression of ISP components is shown in Table 12. For KEGG metabolic pathways, only those containing DEGs from at least 3 out of the 5 overexpression experiments are shown. The KEGG classification shown in Table 11 shows that the ISP regulates carbohydrate, lipid and amino acid metabolism in Drosophila ISP component.

TABLE 2
The Primer Pairs of the Selected Genes for QRT-PCR Verification

| Probe Set | Forward Primer/SEQ ID NO | Reverse Primer/SEQ ID NO |
|---|---|---|
| 141450_at | TCGTTCCGGTGGCATTGT / 1 | GAAGACGCCGCGGATGT / 51 |
| 141454_at | CTGGAGGTGCCCGCATTA / 2 | AATGATTTTCGCGCTGCAA / 52 |
| 141547_at | TCGGCCAATCACCTACCAGTA / 3 | CCGCCGTGCCACTTGTA / 53 |
| 141688_at | GACCTCATGGGCTCCAACAT / 4 | CCTTGGACGATCTCCTTGTTCT / 54 |
| 142165_at | CGGAGTCTTTAATCATAATATGGAAACC / 5 | GCGCGCTCAATGGAAACTA / 55 |
| 142318_at | GCCGCCGAACCAGTTGT / 6 | CCACGACTGGCTGATCCTTAA / 56 |
| 142359_at | CCACGAGTCCCAGCCACTT / 7 | GTCGCCCACATTCGCATATC / 57 |
| 142414_at | CCCTGTCCGGAAGCCAAT / 8 | AGGGTGTCCGCATCGAAGT / 58 |
| 142415_at | CATTTTCCCCGTTTCCTTCTAGT / 9 | GTTTGCGCCTCAACTTAAGCA / 59 |
| 142516_at | CTCTCGTTTCAATCCCAATGCT / 10 | GGGCAGCCTGAGCCTGTT / 60 |
| 143242_at | CGCCAGGGAGAACTCAACAT / 11 | CCCAGGTTGAGAACGATTACATAGA / 61 |
| 143385_at | TTAGCCAGAGCCAGTGTGCTT / 12 | ACCCTGGCAGGCATCCTT / 62 |
| 143443_at | AGGTGTGGACCAGCGACAAT / 13 | CTTTCCAGCTCGGTTCTGAGTT / 63 |
| 143472_at | CAAGCGGATGCCGTTGTC / 14 | TTGGCCCATCCTGAAAATGT / 64 |
| 143604_r_at | TTAGCCAGAGCCAGTGTGCTT / 15 | ACCCTGGCAGGCATCCTT / 65 |
| 144712_at | GGAGCGGTTCATAATCCAGACA /16 | AGGCATTCTTCGTGGACACAA / 66 |
| 146028_at | ACTTCGCTCCGAATCGTGTT / 17 | GTGCTTTAAGGCATCCGGTTT / 67 |
| 146619_i_at | AGCCATCCAGCGTTTCAAGA / 18 | CGCACACGCTCCACGTT / 68 |
| 146809_at | ATCTATAAGGCGCTGGTGGAACT / 19 | CGCTTCACCACAAACACATTCT / 69 |
| 146850_at | GCGTTTCCCATGGACCTCTA / 20 | CCGGCATGAGGCAGAACT / 70 |
| 146898_at | ATGATATCCGGCTTTGTGGAGAT / 21 | CCCAAAACGGCCAGTTTCTT / 71 |
| 147029_s_at | GTTGCCACTCACTTGCTTCGT / 22 | GAATTTTGGCCGGCAGAAT / 72 |
| 147118_at | GCCCAACTACGAGCAGATGAA / 23 | TAAGCAGACCACTAGCTCCATAGGT / 73 |
| 147225_at | CCCTTCGCAAGTATCCAGTTCT / 24 | AACAGTGGTTCCCTTGGCAAT / 74 |
| 147430_at | CTACGGCCGGAAATGTGATT / 25 | CGGCCTACCCGTCCTGTA / 75 |
| 147431_at | CCGGAGAAATCCATCCAGAA / 26 | CGGGCGACAGTGGAACA / 76 |
| 147473_at | GCATATGCTCCCAATTTTGATGT / 27 | GGCTCAGATCGAATCCTTGCT / 77 |
| 147520_at | CAGGATCTGGCTGACTTGCA / 28 | ATGAAACCGCTCTTGACCAGAA / 78 |
| 148253_at | GTGTGGCTACCAATCTTCAGTATGTC / 29 | GCCACGCAGAGGGTTGAGT / 79 |
| 148259_at | TTTCATCCAACGTGGTCACAGT / 30 | TTGCAGTGGCTGGTTCCAT / 80 |
| 148964_at | CAACGACATCTCGCTTATTCGA / 31 | TACGTGGGAAACTGGCCATT / 81 |
| 149233_at | CAGTGGTGATGGTAGCCTGGTA / 32 | GCCAGCCACGCCAAGTAG / 82 |
| 149394_at | CCACGCCTATCAACGAAGCT / 33 | GGCCAAGGGCAAGTCCAT / 83 |
| 150151_at | GAAACCACGCGGGAGATCT / 34 | AATGTCAAGTACTCCTCCTGGAAGA / 84 |
| 150421_at | CTCTTTGGCATCTTCGTTTCAA / 35 | TTTGCCTACCACATAGTTCAGCTT / 85 |
| 150699_at | TATGCTGGCAAGAATGTGAAGAA / 36 | TGGAGCTCAGGCGCTTGT / 86 |
| 151781_at | AGGCATTGAAAACGGTTCGA / 37 | TGCCACAGGCATCAATAGCA / 87 |
| 151788_at | CTGGAGCGCTACAACAAGGAA / 38 | AATATTTTACAGAGCCTGGTGTTAGACA / 88 |
| 151793_at | TCGCTAACACTCTGCAGTACGAA / 39 | AACGCAGATCACGTTGTTGGT / 89 |
| 151932_at | ACCATGTGCTCCACCATCTCTT / 40 | GAGATGACCCTTGAACTCATCGA / 90 |
| 152049_at | CCGAACCGTCCAGGAAGA / 41 | AAGATTCCGCCAGCAACATT / 91 |
| 152142_at | TCTAGCCACCGCAAACGAA / 42 | CCTGTCTGCCTTTGTATAAATGAAATATT / 92 |
| 152245_at | TGTACTCTATTACCTCGAAAGTGGAACT / 43 | CGGACACGATTGCTCTTCAA / 93 |
| 152369_at | GGCTCCGATGCCTCTCTGTA / 44 | TAACCGTCAATGGAGGATCCA / 94 |
| 152456_at | ACGGAGAGGGCGTACGAGTA / 45 | GAAGCCCAGCTTCTCCATCA / 95 |
| 152687_at | CTGATCCAACTGCCAGAACCA /46 | GCCACGCTGCCCACATA / 96 |
| 152833_at | ATTCCGCCCAGAGCGATT / 47 | ACTTCTGGCCTATGCAGTTCCTT / 97 |
| 152843_at | CATCTGCGGAGCACTGTCTCT / 48 | GAAGUCTCCCCATCCTCGAT / 98 |
| 154014_at | GTGGCGGACATGGAGTTCTT / 49 | TCAATTAGCTCCAGAATGCCAAT / 99 |
| 154894_at | AGAAGAAGCGGCATCACGTT / 50 | CACTTCCTCGCATTGCTTGTT / 100 |

TABLE 3
Numbers of Genes Differentially-Regulated
by Overexpression of ISP Components

| Differential Expression | | Up- | Down- |
|---|---|---|---|
| in Response To | Total | regulated | regulated |
| dAkt1 | 128 | 65 | 63 |
| Dp110^{CAAX} | 339 | 254 | 85 |
| Dfoxo | 234 | 101 | 133 |
| dPTEN | 85 | 61 | 24 |
| Dp110^{D954A} | 16 | 11 | 5 |

Note:
Overview of the numbers of DEGs following overexpression of basic ISP genes.

TABLE 4
The Top 20 Down-Regulated and Top 20 Up-Regulated Genes in
Response to dAkt1 Overexpression Ranked by Fold Change

| Probe Set | CG No. | Name | Function | Process | Fold |
|---|---|---|---|---|---|
| 150699_at | CG6295 | — | triacylglycerol lipase | — | −126.53 |
| 142516_at | CG10077 | — | — | — | −73.36 |
| 148228_at | CG10592 | — | alkaline phosphatase | — | −16.1 |
| 151781_at | CG5107 | — | — | — | −13.42 |
| 143443_at | CG12763 | Dpt | antibacterial peptide, gram- | antibacterial humoral | −12.07 |
| | | | negative antibacterial peptide | response (sensu | |
| | | | | Invertebrata) | |
| 146569_at | CG9259 | — | — | — | −11.86 |
| 147150_at | CG4740 | AttC | antibacterial peptide | antibacterial humoral | −10.95 |
| | | | | response (sensu | |
| | | | | Invertebrata) | |
| 150482_at | CG13607 | — | — | — | −8.59 |
| 142762_at | CG13903 | — | ubiquitin-specific protease | deubiquitination | −7.1 |
| 144749_at | CG2309 | — | protein serine/threonine | protein amino acid | −6.82 |
| | | | kinase, MAP kinase kinase | phosphorylation | |
| 151361_at | CG17015 | RhoGAP18B | — | — | −6.48 |
| 147118_at | CG12374 | — | carboxypeptidase A | — | −5.89 |
| 152870_at | CG6716 | prd | DNA binding, specific RNA | periodic partitioning | −5.6 |
| | | | polymerase II transcription | by pair rule gene | |
| | | | factor | | |
| 144565_at | CG6067 | — | — | — | −5.38 |
| 141534_at | CG3961 | — | long-chain-fatty-acid-CoA- | — | −5.05 |
| | | | ligase | | |
| 152313_at | CG10241 | Cyp6a17 | cytochrome P450 | — | −4.93 |
| 147144_at | CG4734 | — | — | — | −4.54 |
| 141300_at | CG9181 | Ptp61F | protein tyrosine phosphatase | axon | −4.5 |
| | | | | guidance\|protein | |
| | | | | amino acid | |
| | | | | dephosphorylation | |
| 141681_at | CG1691 | Imp | mRNA binding | — | −4.46 |
| 148964_at | CG7542 | — | chymotrypsin | — | −3.95 |
| 152833_at | CG1944 | Cyp4p2 | cytochrome P450 | — | 3.67 |
| 142279_at | CG17632 | bw | eye pigment precursor | eye pigment | 3.74 |
| | | | transporter, ATP-binding | biosynthesis\|pteridine | |
| | | | cassette (ABC) transporter | biosynthesis | |
| 152078_at | CG9547 | — | glutaryl-CoA dehydrogenase | — | 3.85 |
| 149303_at | CG14661 | — | — | — | 3.97 |
| 152687_at | CG8952 | — | serine-type endopeptidase | — | 3.97 |
| 152245_at | CG6183 | LOC156106 | — | — | 3.99 |
| 143008_at | CG8987 | tam | 3′-5′exodeoxyribonuclease; | DNA dependent | 4.05 |
| | | | gamma DNA-directed DNA | DNA replication | |
| | | | polymerase | | |
| 148103_at | CG10812 | — | defense/immunity protein | defense response | 4.05 |
| 152128_at | CG9761 | Nep2 | endothelin-converting enzyme; | — | 4.38 |
| | | | metallopeptidase | | |
| 150369_at | CG18594 | — | — | — | 4.39 |
| 150337_at | CG6560 | — | ARF small monomeric GTPase | — | 4.6 |
| 151927_at | CG4784 | — | — | — | 4.81 |
| 150154_at | CG7698 | — | — | mRNA | 5.43 |
| | | | | cleavage\|mRNA | |
| | | | | polyadenylation | |
| 147818_at | CG13565 | similar to | — | — | 5.87 |
| | | CG13565 | | | |
| 142217_at | CG2655 | HLH3B | transcription factor | — | 6.77 |
| 152456_at | CG3318 | Dat | arylalkylamine | catecholamine | 8.36 |
| | | | N-acetyltransferase; arylamine | metabolism | |
| | | | N-acetyltransferase | | |
| 144619_at | CG4547 | — | — | — | 8.95 |
| 141547_at | CG12529 | Zw | glucose-6-phosphate 1- | pentose-phosphate | 9.93 |
| | | | dehydrogenase | shunt | |
| 154985_at | CG9083 | — | — | — | 10.95 |
| 147659_at | CG13504 | — | — | — | 24.48 |

Note:
The listed DEGs are depicted by Affymetrix probe set, CG number, gene description including gene name, molecular function and biological process information.

TABLE 5
The Top 20 Down-Regulated and Top 20 Up-Regulated Genes in Response
to Dp110^{D954A} Overexpression Ranked by Fold Change

| Probe Set | CG No. | Name | Function | Process | Fold |
|---|---|---|---|---|---|
| 146809_at | CG11669 | — | alpha (α)-glucosidase | — | −22.58 |
| 147812_at | CG5569 | — | — | — | −17.59 |
| 150699_at | CG6295 | — | triacylglycerol lipase | — | −17.16 |
| 148228_at | CG10592 | — | alkaline phosphatase | — | −15.83 |
| 150702_at | CG6283 | — | triacylglycerol lipase | — | −15.32 |
| 142359_at | CG8689 | — | — | — | −11.94 |
| 148253_at | CG6467 | — | translation elongation | translational | −11.83 |
| | | | factor\|protein-synthesizing | elongation | |
| | | | GTPase, elongation serine- | | |
| | | | type endopeptidase | | |
| 150588_at | CG11909 | — | α-glucosidase | — | −10.33 |
| 144310_at | CG8997 | — | — | — | −8.63 |
| 152313_at | CG10241 | Cyp6a17 | cytochrome P450 | — | −8.22 |
| 143604_r_at | CG12351 | deltaTry | trypsin | proteolysis and | −7.93 |
| | | | | peptidolysis | |
| 152870_at | CG6716 | prd | DNA binding, specific RNA | periodic partitioning | −7.27 |
| | | | polymerase II transcription | by pair rule gene | |
| | | | factor | | |
| 143242_at | CG8696 | LvpH | α-glucosidase | glucose metabolism | −7.2 |
| 142201_at | CG4363 | — | — | — | −6.96 |
| 147459_at | CG18609 | — | — | — | −6.61 |
| 149024_at | CG3819 | — | — | — | −6.13 |
| 149922_at | CG3610 | — | — | — | −5.96 |
| 144565_at | CG6067 | — | — | — | −5.91 |
| 144350_at | CG5254 | — | carrier tricarboxylate carrier | — | −5.44 |
| 146810_at | CG8690 | — | α-glucosidase | — | −5.43 |
| 148666_at | CG10522 | — | protein serine/threonine kinase | protein amino acid | 6.78 |
| | | | diacylglycerol binding | phosphorylation | |
| 150810_at | CG11898 | — | multidrug transporter | — | 6.86 |
| | | | xenobiotic-transporting | | |
| | | | ATPase | | |
| 150337_at | CG6560 | — | ARF small monomeric GTPase | — | 7.05 |
| 154795_at | CG14231 | — | — | — | 7.3 |
| 143989_at | CG3480 | — | — | — | 7.38 |
| 152456_at | CG3318 | Dat | arylalkylamine | catecholamine | 7.4 |
| | | | N-acetyltransferase, arylamine | metabolism | |
| | | | N-acetyltransferase | | |
| 149791_at | CG6753 | — | triacylglycerol lipase | — | 8.05 |
| 148640_at | CG11529 | — | serine-type endopeptidase | — | 8.62 |
| 152749_at | CG15772 | — | — | — | 9.62 |
| 144196_at | CG6743 | clone | — | — | 10.11 |
| | | LD18761 | | | |
| | | BcDNA | | | |
| | | mRNA | | | |
| 153258_at | CG4860 | — | acyl-CoA dehydrogenase | — | 10.73 |
| 150219_at | CG11453 | — | long-chain fatty acid | — | 11.34 |
| | | | transporter | | |
| 154291_at | CG1405 | — | ATP-dependent helicase, ATP- | mRNA splicing | 11.75 |
| | | | dependent RNA helicase, | | |
| | | | pre-mRNA splicing factor | | |
| 144619_at | CG4547 | — | — | — | 13.51 |
| 153910_at | CG8211 | — | — | — | 15.05 |
| 154429_at | CG2054 | Cht2 | chitinase | cuticle chitin | 16.05 |
| | | | | catabolism | |
| 147818_at | CG13565 | similar to | — | — | 18.18 |
| | | CG13565 | | | |
| 146141_at | CG5375 | — | — | — | 20.39 |
| 146780_at | CG2916 | Sep5 | GTPase | cytokinesis | 20.91 |
| 145121_at | CG6299 | — | glycolipid transfer | — | 46.54 |

Note:
The listed DEGs are depicted by Affymetrix probe set, CG number, gene description including gene name, molecular function and biological process information.

TABLE 6
The DEGs in Response to Dp110^{D954A} Overexpression

| Probe Set | CG No. | Name | Function | Process | Fold |
|---|---|---|---|---|---|
| 150699_at | CG6295 | — | triacylglycerol lipase | — | −10.69 |
| 148539_at | CG18348 | — | — | — | −4.67 |
| 148553_at | CG6261 | — | — | — | −4.13 |
| 152313_at | CG10241 | Cyp6a17 | cytochrome P450 | — | −2.85 |
| 147520_at | CG13873 | Obp56g | odorant binding | — | −2.48 |
| 146273_at | CG16997 | — | serine-type endopeptidase | — | −2.31 |
| 145474_at | CG1678 | — | — | — | −2.03 |
| 152918_at | CG1114 | Hph | — | — | 2.19 |
| 142415_at | CG3798 | Nmda1 | N-methyl-D-aspartate selective | — | 2.54 |
| | | | glutamate receptor | | |
| 152142_at | CG9175 | — | DNA binding | — | 2.76 |
| 148259_at | CG6602 | — | — | — | 2.9 |
| 144167_at | CG4325 | — | — | — | 3 |
| 148948_at | CG3882 | — | — | — | 5.35 |
| 150487_at | CG13608 | mRpS24 | structural constituent of | protein biosynthesis | 6.69 |
| | | | ribosome | | |
| 151818_at | CG7051 | — | dynein ATPase; motor | microtubule-based | 9.62 |
| | | | | movement | |
| 152833_at | CG1944 | Cyp4p2 | cytochrome P450 | — | 12.24 |

Note:
The listed DEGs are depicted by Affymetrix probe set, CG number, gene description including gene name, molecular function and biological process information.

TABLE 7
The Top 20 Down-Regulated and Top 20 Up-Regulated Genes in
Response to dPTEN Over-Expression Ranked by Fold Change

| Probe Set | CG No. | Name | Function | Process | Fold |
|---|---|---|---|---|---|
| 150699_at | CG6295 | — | triacylglycerol lipase | — | −11.73 |
| 147817_at | CG2812 | — | heterotrimeric G-protein | — | −6.91 |
| | | | GTPase, β-subunit | | |
| 148539_at | CG18348 | — | — | — | −5.68 |
| 149329_at | CG2663 | — | tocopherol binding | — | −5.55 |
| 152794_at | CG8560 | — | NOT carboxypeptidase | — | −4.63 |
| 146587_at | CG17571 | similar to | serine-type endopeptidase | — | −4.34 |
| | | CG17571 | | | |
| 152049_at | CG2668 | clone GH06048 | — | — | −3.77 |
| | | BcDNA.GH0 | | | |
| | | 6048 mRNA | | | |
| 147520_at | CG13873 | Obp56g | odorant binding | — | −3.76 |
| 142414_at | CG11315 | similar to | — | — | −3.43 |
| | | CG11315 | | | |
| 146619_i_at | CG12628 | — | — | — | −3.39 |
| 148553_at | CG6261 | — | — | — | −3.36 |
| 146352_r_at | CG16885 | — | — | — | −3.29 |
| 148964_at | CG7542 | — | chymotrypsin | — | −3.2 |
| 146660_at | CG7882 | — | glucose transporter | — | −3.11 |
| 143385_at | CG18444 | alphaTry | trypsin | proteolysis and | −2.77 |
| | | | | peptidolysis | |
| 147118_at | CG12374 | — | carboxypeptidase A | — | −2.75 |
| 146620_s_at | CG12628 | Mgstl | glutathione transferase | — | −2.42 |
| 152369_at | CG13095 | — | aspartic-type endopeptidase | — | −2.4 |
| 141450_at | CG1982 | Sodh-1 | L-iditol 2-dehydrogenase | — | −2.39 |
| 145474_at | CG1678 | — | — | — | −2.34 |
| 146814_at | CG12780 | — | gram-negative bacterial | — | 3.3 |
| | | | binding | | |
| 144234_at | CG7578 | — | ARF guanyl-nucleotide | ER to Golgi | 3.31 |
| | | | exchange factor | transport\|intra-Golgi | |
| | | | | transport | |
| 153902_at | CG3385 | nvy | transcription factor | — | 3.35 |
| 141454_at | CG13213 | — | — | — | 3.45 |
| 152940_at | CG9663 | — | ATP-binding cassette (ABC) | — | 3.54 |
| | | | transporter | | |
| 142522_at | CG12345 | Cha | choline O-acetyltransferase | acetylcholine | 3.57 |
| | | | | biosynthesis | |
| 148102_at | CG10813 | — | defense/immunity protein | defense response | 3.59 |
| 141205_at | CG18177 | — | — | — | 3.63 |
| 151748_at | CG7450 | — | — | — | 3.65 |
| 154894_at | CG8374 | dmt | — | — | 3.68 |
| 151127_f_at | CG12566 | — | — | — | 3.88 |
| 154985_at | CG9083 | — | — | — | 3.89 |
| 142797_at | CG5460 | H | transcription co-repressor | sensory organ | 3.9 |
| | | | | determination | |
| 150683_at | CG6403 | — | — | — | 3.99 |
| 143566_i_at | CG2163 | Pabp2 | RNA binding\|poly(A) binding | mRNA | 4.13 |
| | | | | polyadenylation | |
| 141831_at | CG3625 | — | — | — | 4.71 |
| 153804_at | CG11881 | — | — | — | 6.06 |
| 143410_at | CG2759 | w | eye pigment precursor | eye pigment | 6.19 |
| | | | transporter; ABC transporter | biosynthesis\|eye | |
| | | | | pigment precursor | |
| | | | | transport | |
| 142225_at | CG18381 | — | — | — | 7.11 |
| 145061_at | CG5228 | — | — | — | 8.05 |

Note:
The listed DEGs are depicted by Affymetrix probe set, CG number, gene description including gene name, molecular function and biological process information.

TABLE 8
The Top 20 Down-Regulated and Top 20 Up-Regulated Genes in
Response to dfoxo Overexpression Ranked by Fold Change

| Probe Set | CG No. | Name | Function | Process | Fold |
|---|---|---|---|---|---|
| 148253_at | CG6467 | — | serine-type endopeptidase | — | −132.5 |
| 150699_at | CG6295 | — | triacylglycerol lipase | — | −112.51 |
| 143604_r_at | CG12351 | deltaTry | trypsin | proteolysis and | −101.85 |
| | | | | peptidolysis | |
| 153069_at | CG15096 | — | high affinity inorganic | — | −33.36 |
| | | | phosphate: sodium symporter | | |
| 144310_at | CG8997 | — | — | — | −31.67 |
| 148228_at | CG10592 | — | alkaline phosphatase | — | −28.02 |
| 143984_at | CG4605 | Acp32CD | — | — | −22.09 |
| 147118_at | CG12374 | — | carboxypeptidase A | — | −18.26 |
| 142419_at | CG12057 | — | — | — | −17.98 |
| 151781_at | CG5107 | — | — | — | −15.11 |
| 147520_at | CG13873 | Obp56g | odorant binding | — | −14.18 |
| 146902_at | CG1652 | lectin-46Cb | galactose binding lectin | — | −13.32 |
| 141435_at | CG11911 | — | serine-type endopeptidase | — | −11.67 |
| 146569_at | CG9259 | — | — | — | −11.21 |
| 149024_at | CG3819 | — | — | — | −10.94 |
| 150574_at | CG11891 | LOC156877 | — | — | −10.88 |
| 142407_at | CG4753 | — | 1-acylglycerol-3-phosphate | — | −10.64 |
| | | | O-acyltransferase | | |
| 150575_at | CG11878 | — | — | — | −10.18 |
| 151793_at | CG6483 | — | serine-type endopeptidase | — | −10.03 |
| 146524_at | CG13965 | — | — | — | −9.98 |
| 144201_at | CG14902 | decay | caspase-3; effector caspase; | apoptosis\|apoptotic | 6.89 |
| | | | caspase translation initiation | program | |
| | | | factor | | |
| 141744_at | CG5772 | Sur | sulfonylurea receptor | — | 7.05 |
| 151670_at | CG2678 | — | — | — | 7.11 |
| 154760_at | CG4618 | — | — | — | 7.24 |
| 153910_at | CG8211 | — | — | — | 7.28 |
| 146413_at | CG6870 | — | electron transporter | — | 7.57 |
| 151899_at | CG11796 | — | 4-hydroxy-phenylpyruvate | — | 7.89 |
| | | | dioxygenase | | |
| 146180_at | CG17107 | similar to | — | — | 8.04 |
| | | CG17107 | | | |
| 151818_at | CG7051 | — | dynein ATPase\|motor | microtubule-based | 8.55 |
| | | | | movement | |
| 150683_at | CG6403 | — | — | — | 9.17 |
| 146850_at | CG13748 | — | serine protease inhibitor | — | 9.18 |
| 152279_at | CG6128 | — | α-L-fucosidase | O-glycoside | 9.93 |
| | | | | catabolism\|fucose | |
| | | | | metabolism | |
| 150711_at | CG17189 | — | — | — | 10.31 |
| 145061_at | CG5228 | — | — | — | 11.43 |
| 150551_at | CG11839 | — | — | — | 12.05 |
| 149394_at | CG15189 | — | — | — | 12.98 |
| 142318_at | CG17919 | — | phosphatidylethanolamine | — | 13.16 |
| | | | binding | | |
| 150552_at | CG9996 | — | — | — | 21.62 |
| 146990_at | CG13215 | — | — | — | 28.09 |
| 154014_at | CG3838 | — | — | — | 34.58 |

Note:
The listed DEGs are depicted by Affymetrix probe set, CG number, gene description including gene name, molecular function and biological process information.

TABLE 9
Selected Biological Process Classification of Genes Differentially-
Regulated Following Overexpression of ISP Components Using GO

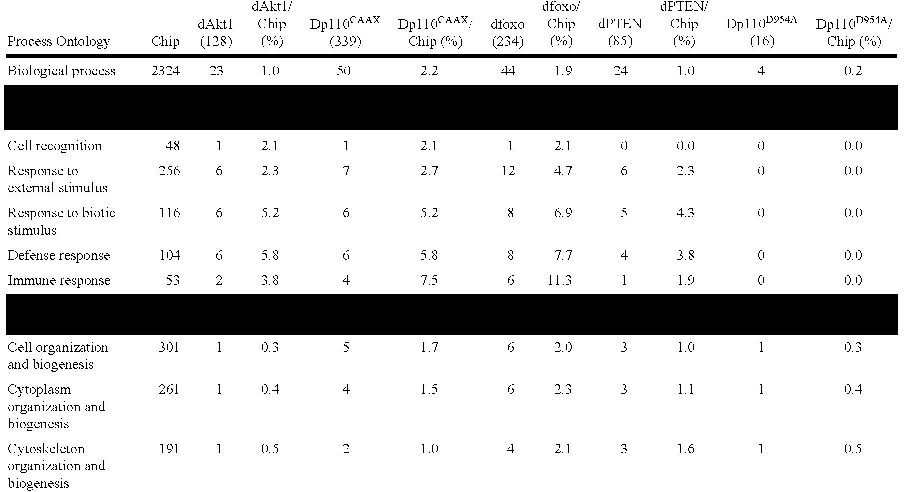

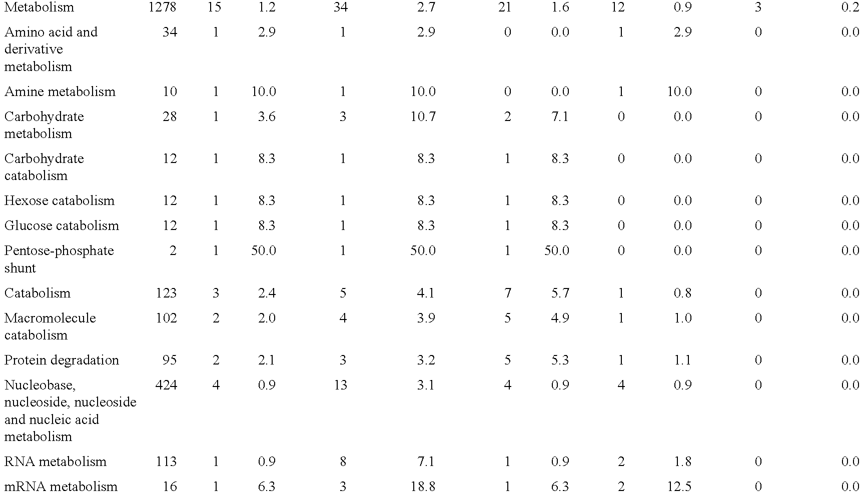

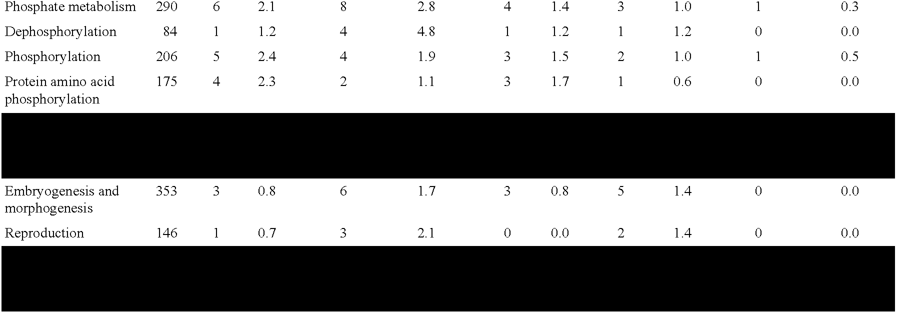

Note:
DEGs following overexpression of ISP components grouped according to GO biological process classes.
Columns from left to right:
(1) primary GO terms (in purple) under the GO term “biological process” and selected secondary GO terms showing more detailed annotation of the primary terms; and
(2) numbers of genes represented on the entire chip in each GO category.
Columns 3, 5, 7, 9 and 11 represent numbers of DEGs following overexpression of dAkt1, Dp110^{CAAX}, dfoxo, dPTEN and Dp110^{D954A}, respectively, in each GO category.
Numbers in the parenthesis in the column headers represent the total number of genes that were differentially-regulated following overexpression of each ISP gene.
Columns 4, 6, 8, 10 and 12 percentage of DEGs for each GO category relative to the total number of genes in that GO category represented on the chip for each ISP gene overexpression.

TABLE 10
Selected Molecular Function Classification of Genes Differentially-
Regulated Following Overexpression of ISP Components Using GO
One gene product may belong to more than one categories.

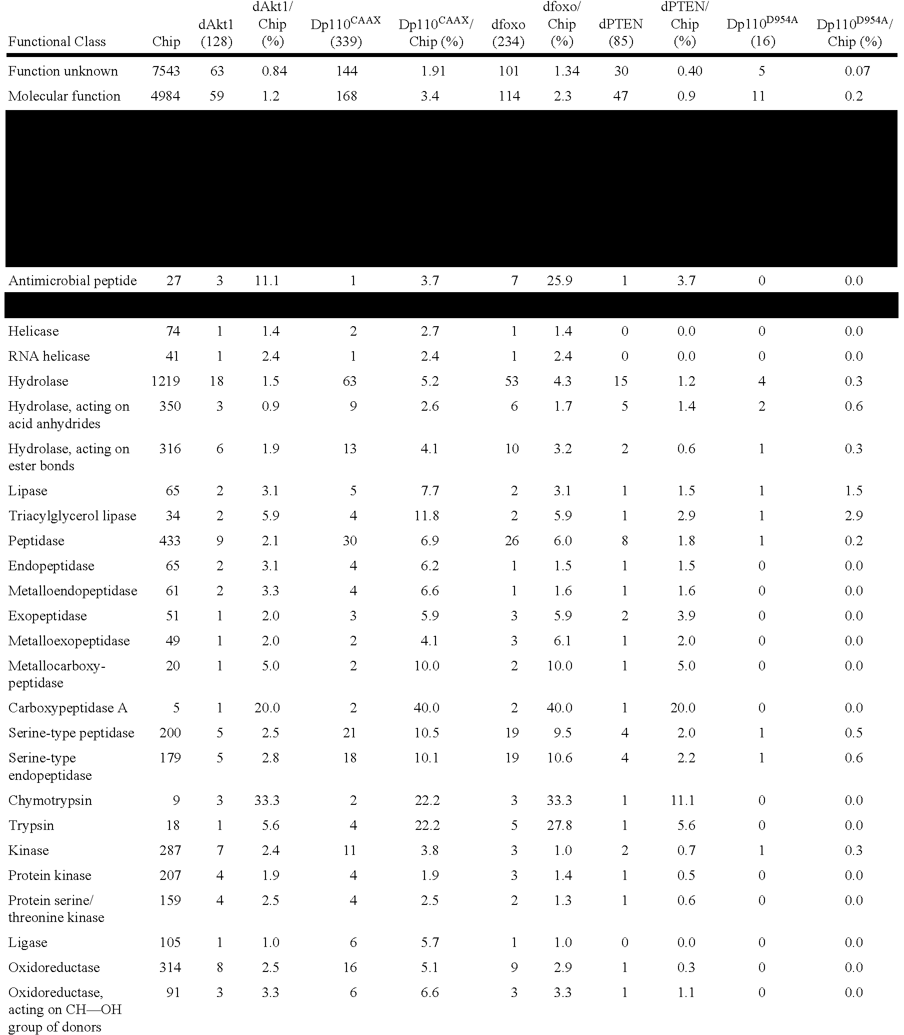

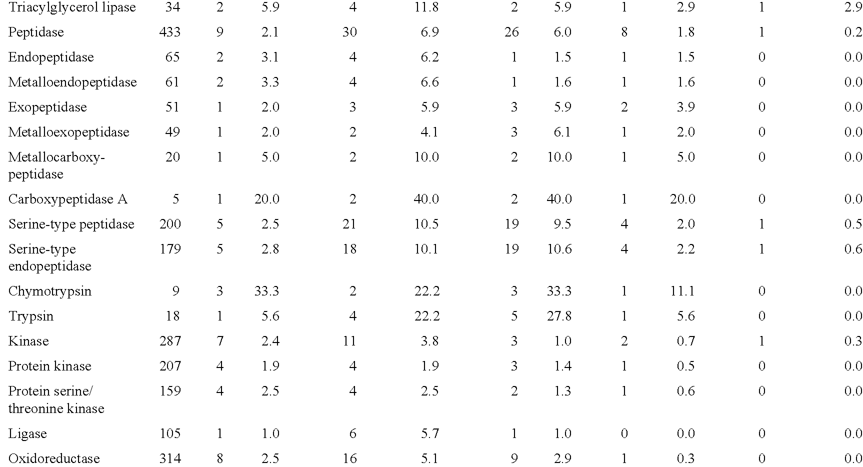

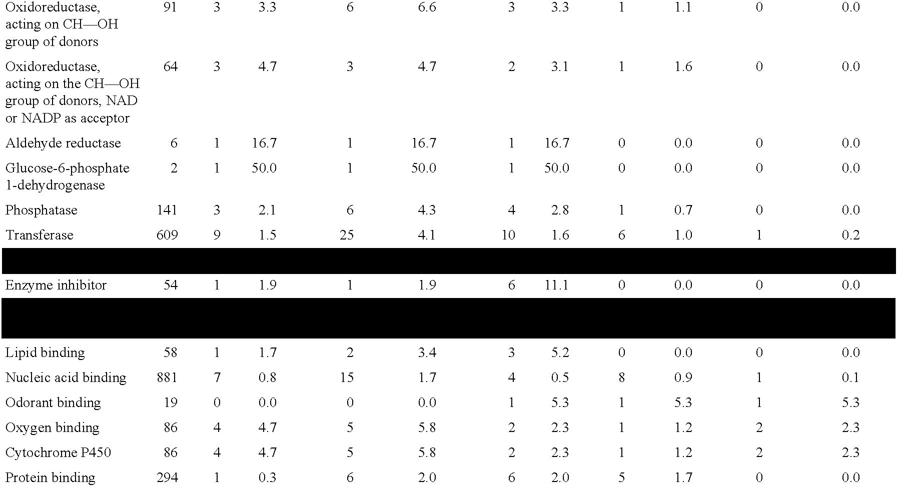

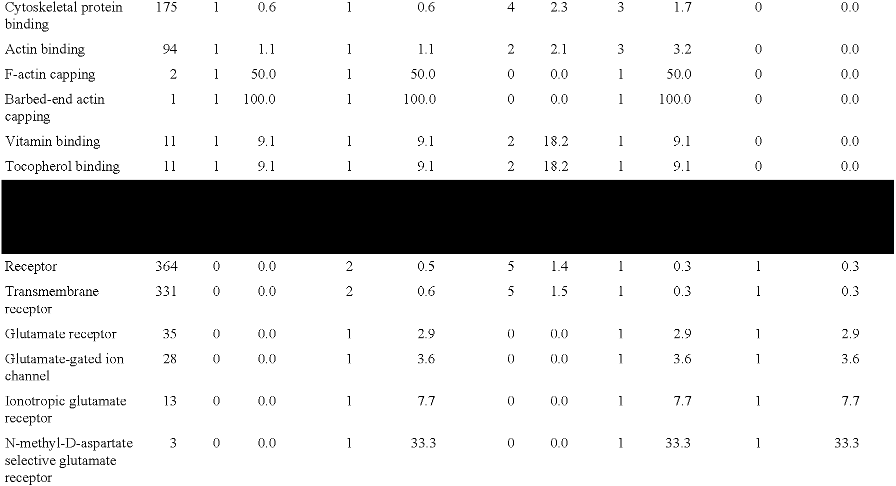

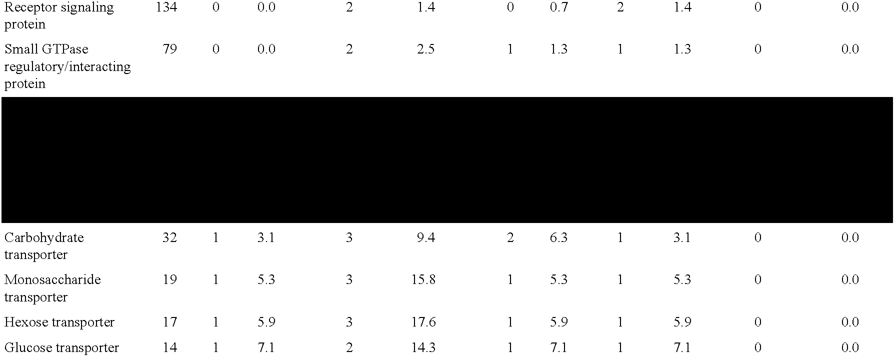

Note:
DEGs following overexpression of ISP components, grouped according to GO molecular function classes.
Columns from left to right:
(1) primary GO terms (in purple) under the GO term “molecular function” and selected secondary GO terms showing more detailed annotation of the primary terms; and
(2) numbers of genes represented on the entire chip in each GO category.
Columns 3, 5, 7, 9 and 11 represent numbers of DEGs following overexpression of dAKT1, Dp100^{CAAX}, dfoxo, dPTEN and Dp110^{D954A}, respectively, in each GO category.
Numbers in the parenthesis in the column headers represent the total number of genes that were differentially-regulated following overexpression of each ISP gene.
Columns 4, 6, 8, 10 and 12 represent percentage of genes that were differentially-regulated for each GO category relative to the total number of genes in that GO category represented on the chip for each ISP gene overexpression.

TABLE 11
DEGs Encoding Products as Classified as Defense/Immunity Proteins According to GO

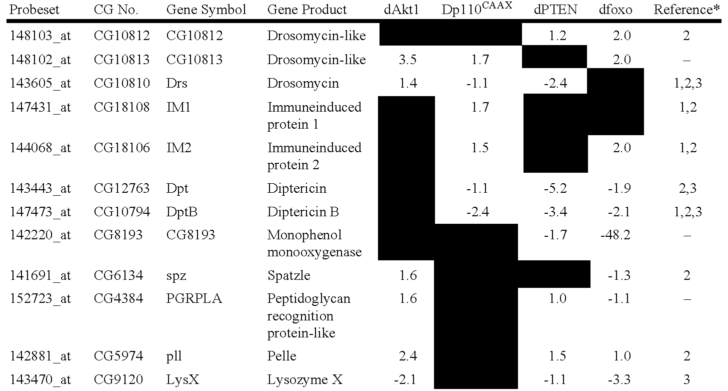

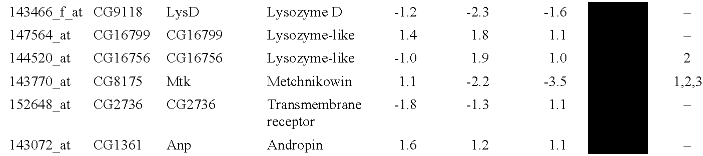

Summary of DEGs encoding products that are classified as defense response proteins according to GO biological process and molecular function classes.
Their corresponding average fold changes following overexpression of insulin signaling pathway components are shown.
Numbers in Green represent fold changes of DEGs in the corresponding experiments.
Numbers not highlighted are the fold changes of genes not satisfying the criteria of differential expression (see Materials and Methods) even though their fold changes may be greater than 2.
*References: 1) Boutros et al. (2002); 2) De Gregorio et al., (2001); and 3) Irving et al. (2001).

TABLE 12
Selected Metabolic Pathway Classification of Genes Differentially-Regulated Following Overexpression of ISP Components Using KEGG

| KEGG | KEGG | CGs in | | dAkt1/ | | | | dfoxo/ | | dPTEN/ | | |
|---|---|---|---|---|---|---|---|---|---|---|---|---|
| Primary | Secondary | the | dAkt1 | Path | Dp110^{CAAX} | Dp110^{CAAX} | dfoxo | Path | dPTEN | Path | Dp110^{D954A} | Dp110^{D954A} |
| Path | Path | Pathway | (128) | (%) | (339) | Path (%) | (234) | (%) | (85) | (%) | (16) | Path (%) |
| Amino | Phenyl- | 24 | 2 | 8.3 | 2 | 8.3 | 1 | 4.2 | 0 | 0.0 | 0 | 0.0 |
| acid | alanine | | | | | | | | | | | |
| metabo- | metabolism | | | | | | | | | | | |
| lism | | | | | | | | | | | | |
| | Tryptophan | 51 | 4 | 7.8 | 4 | 7.8 | 1 | 2.0 | 1 | 2.0 | 1 | 2.0 |
| | metabolism | | | | | | | | | | | |
| Carbo- | Galactose | 18 | 1 | 5.6 | 4 | 22.2 | 1 | 5.6 | 0 | 0.0 | 0 | 0.0 |
| hydrate | metabolism | | | | | | | | | | | |
| metabo- | | | | | | | | | | | | |
| lism | | | | | | | | | | | | |
| | Pentose | 18 | 2 | 11.1 | 1 | 5.6 | 3 | 16.7 | 0 | 0.0 | 0 | 0.0 |
| | phosphate | | | | | | | | | | | |
| | pathway | | | | | | | | | | | |
| Lipid | Fatty acid | 48 | 4 | 8.3 | 5 | 10.4 | 1 | 2.1 | 1 | 2.1 | 1 | 2.1 |
| metabo- | metabolism | | | | | | | | | | | |
| lism | | | | | | | | | | | | |
| Metabo- | Folate | 20 | 1 | 5.0 | 2 | 10.0 | 2 | 10.0 | 0 | 0.0 | 0 | 0.0 |
| lism | biosynthesis | | | | | | | | | | | |
| of co- | | | | | | | | | | | | |
| factors | | | | | | | | | | | | |
| and | | | | | | | | | | | | |
| vitamins | | | | | | | | | | | | |
| Metabo- | Glycero-lipid | 57 | 1 | 1.8 | 2 | 3.5 | 2 | 3.5 | 1 | 1.8 | 0 | 0.0 |
| lism of | metabolism | | | | | | | | | | | |
| complex | | | | | | | | | | | | |
| lipids | | | | | | | | | | | | |
| | Sphingogly | 25 | 2 | 8.0 | 1 | 4.0 | 1 | 4.0 | 0 | 0.0 | 0 | 0.0 |
| | co-lipid | | | | | | | | | | | |
| | metabolism | | | | | | | | | | | |
| Metabo- | Glutathione | 25 | 1 | 4.0 | 4 | 16.0 | 3 | 12.0 | 3 | 12.0 | 0 | 0.0 |
| lism of | metabolism | | | | | | | | | | | |
| other | | | | | | | | | | | | |
| amino | | | | | | | | | | | | |
| acids | | | | | | | | | | | | |
| Nucleo- | Purine | 65 | 2 | 3.1 | 4 | 6.2 | 2 | 3.1 | 0 | 0.0 | 0 | 0.0 |
| tide | metabolism | | | | | | | | | | | |
| metabo- | | | | | | | | | | | | |
| lism | | | | | | | | | | | | |

DEGs following overexpression of ISP components, grouped according to KEGG.
Columns from left to right: (1) KEGG primary path, (2) KEGG secondary path and (3) numbers of genes belonging to that secondary pathway.
Columns 4, 6, 8, 10 and 12 represent numbers of DEGs following overexpression of dAkt1, Dp110^{CAAX}, dfoxo, dPTEN and Dp110^{D954A}, respectively, in each KEGG category. Numbers in the parenthesis in the column headers represent the total number of genes that were differentially-regulated following overexpression of each ISP gene.
Columns 5, 7, 9, 11 and 13 represent percentage of genes that were differentially-regulated for each KEGG category relative to the total number of genes in that KEGG category for each ISP gene overexpression.

TABLE 13
Human Homologs of Drosophila Genes Regulated by Insulin Signaling Pathway

| Human Gene | FlyBase | FlyGene | Blast | Blast | Pct | Affy Chip |
|---|---|---|---|---|---|---|
| Locus ID | Gene ID | CG | Score | Prob | Ident | Probe Set |
| LocusLink: 10013 | FBgn0026428 | CG6170 | 687 | 0 | 45 | 155023_at |
| LocusLink: 10109 | FBgn0032859 | CG10954 | 439 | 1E−123 | 75 | 154503_at |
| LocusLink: 10113 | FBgn0031779 | CG9175 | 205 | 4.3E−53 | 32 | 152142_at |
| LocusLink: 102 | FBgn0015954 | CG7147 | 453 | 5E−127 | 41 | 141542_at |
| LocusLink: 10247 | FBgn0028510 | CG15261 | 139 | 5.7E−34 | 46 | 144220_at |
| LocusLink: 1036 | FBgn0034364 | CG5493 | 213 | 1.1E−55 | 52 | 147450_at |
| LocusLink: 10564 | FBgn0028538 | CG7578 | 1981 | 0 | 59 | 144234_at |
| LocusLink: 10565 | FBgn0028538 | CG7578 | 1769 | 0 | 62 | 144234_at |
| LocusLink: 10606 | FBgn0020513 | CG3989 | 491 | 5E−139 | 58 | 153428_at |
| LocusLink: 10797 | FBgn0010222 | CG18466 | 339 | 1.9E−93 | 56 | 151767_at |
| LocusLink: 10898 | FBgn0015621 | CG3642 | 322 | 2.4E−88 | 54 | 143833_at |
| LocusLink: 10946 | FBgn0014366 | CG2925 | 626 | 1E−179 | 63 | 154122_at |
| LocusLink: 10981 | FBgn0015788 | CG8024 | 288 | 1.9E−78 | 67 | 153524_at |
| LocusLink: 1103 | FBgn0000303 | CG12345 | 456 | 2E−128 | 38 | 142522_at |
| LocusLink: 11140 | FBgn0011573 | CG12019 | 327 | 1.1E−89 | 52 | 143662_at |
| LocusLink: 11143 | FBgn0031911 | CG5229 | 473 | 2E−133 | 50 | 153634_at |
| LocusLink: 1118 | FBgn0022702 | CG2054 | 246 | 1.9E−65 | 34 | 154429_at |
| LocusLink: 11182 | FBgn0031517 | CG15406 | 141 | 8.3E−34 | 26 | 145698_at |
| LocusLink: 116064 | FBgn0030209 | CG2892 | 171 | 2.3E−43 | 49 | 144806_at |
| LocusLink: 123169 | FBgn0019637 | CG1433 | 285 | 7.6E−77 | 56 | 154540_at |
| LocusLink: 124936 | FBgn0030099 | CG12056 | 156 | 2.2E−38 | 38 | 142420_at |
| LocusLink: 1360 | FBgn0032144 | CG17633 | 234 | 1.1E−61 | 33 | 146103_at |
| LocusLink: 1360 | FBgn0032144 | CG17633 | 234 | 1.1E−61 | 33 | 146103_at |
| LocusLink: 145226 | FBgn0033205 | CG2064 | 256 | 1.7E−68 | 47 | 141701_at |
| LocusLink: 7678 | FBgn0030010 | CG10959 | 185 | 4.6E−47 | 37 | 153749_at |
| LocusLink: 1510 | FBgn0032049 | CG13095 | 304 | 5.5E−83 | 45 | 152369_at |
| LocusLink: 1653 | FBgn0015075 | CG9054 | 872 | 0 | 58 | 141634_at |
| LocusLink: 1803 | FBgn0031741 | CG11034 | 396 | 4E−110 | 33 | 145847_at |
| LocusLink: 189 | FBgn0014031 | CG3926 | 173 | 5.7E−44 | 48 | 153046_at |
| LocusLink: 2052 | FBgn0034406 | CG15106 | 317 | 8.3E−87 | 38 | 154747_at |
| LocusLink: 217 | FBgn0032114 | CG3752 | 723 | 0 | 68 | 146084_at |
| LocusLink: 2191 | FBgn0031741 | CG11034 | 361 | 9E−100 | 31 | 145847_at |
| LocusLink: 2194 | FBgn0027571 | CG3523 | 1077 | 0 | 48 | 152126_at |
| LocusLink: 64137 | FBgn0031220 | CG4822 | 354 | 1E−117 | 40 | 143011_at |
| LocusLink: 2241 | FBgn0000723 | CG8874 | 523 | 1E−148 | 37 | 143164_at |
| LocusLink: 225689 | FBgn0030119 | CG2309 | 281 | 5.3E−76 | 50 | 144749_at |
| LocusLink: 22845 | FBgn0034141 | CG8311 | 147 | 1.6E−35 | 31 | 142795_at |
| LocusLink: 22858 | FBgn0032164 | CG4588 | 438 | 8E−123 | 65 | 146112_at |
| LocusLink: 22903 | FBgn0031098 | CG17068 | 137 | 1.9E−32 | 38 | 141427_at |
| LocusLink: 22911 | FBgn0034931 | CG2812 | 358 | 3E−99 | 52 | 147817_at |
| LocusLink: 22938 | FBgn0004856 | CG8264 | 521 | 5E−148 | 62 | 142686_at |
| LocusLink: 23080 | FBgn0030499 | CG11178 | 200 | 3.1E−51 | 30 | 153940_at |
| LocusLink: 231 | FBgn0033101 | CG9436 | 265 | 2.2E−71 | 44 | 142716_at |
| LocusLink: 23205 | FBgn0027348 | CG4501 | 474 | 6E−134 | 41 | 153318_at |
| LocusLink: 23762 | FBgn0020626 | CG6708 | 578 | 5E−165 | 44 | 154587_at |
| LocusLink: 246213 | FBgn0034394 | CG15096 | 133 | 1.2E−31 | 33 | 153069_at |
| LocusLink: 249 | FBgn0033423 | CG1809 | 351 | 5.8E−97 | 44 | 151956_at |
| LocusLink: 2539 | FBgn0004057 | CG12529 | 658 | 0 | 65 | 141547_at |
| LocusLink: 25766 | FBgn0031492 | CG3542 | 325 | 7.5E−89 | 52 | 154288_at |
| LocusLink: 2589 | FBgn0027558 | CG4445 | 440 | 1E−123 | 56 | 152222_at |
| LocusLink: 26085 | FBgn0031249 | CG11911 | 134 | 8.5E−32 | 34 | 141435_at |
| LocusLink: 2618 | FBgn0000053 | CG8761 | 597 | 2E−170 | 47 | 143062_at |
| LocusLink: 2639 | FBgn0031824 | CG9547 | 572 | 1E−163 | 68 | 152078_at |
| LocusLink: 2643 | FBgn0003162 | CG9441 | 259 | 1.3E−69 | 75 | 154812_at |
| LocusLink: 26503 | FBgn0031645 | CG3036 | 299 | 3.2E−81 | 36 | 151989_at |
| LocusLink: 27019 | FBgn0035100 | CG7051 | 143 | 5E−34 | 27 | 151818_at |
| LocusLink: 27244 | FBgn0034897 | CG11299 | 346 | 2.3E−95 | 43 | 154871_at |
| LocusLink: 27294 | FBgn0031417 | CG3597 | 220 | 1.2E−57 | 38 | 145636_at |
| LocusLink: 2752 | FBgn0001142 | CG2718 | 453 | 1E−127 | 58 | 141257_at |
| LocusLink: 2762 | FBgn0031661 | CG8890 | 526 | 1E−149 | 70 | 153281_at |
| LocusLink: 2764 | FBgn0028894 | CG5869 | 159 | 6.9E−40 | 53 | 141567_at |
| LocusLink: 28976 | FBgn0033637 | CG9006 | 197 | 2.4E−50 | 28 | 153329_at |
| LocusLink: 28996 | FBgn0035142 | CG17090 | 468 | 7E−132 | 55 | 151694_at |
| LocusLink: 2931 | FBgn0003371 | CG2621 | 582 | 4E−166 | 78 | 143343_i_at |
| LocusLink: 2932 | FBgn0003371 | CG2621 | 589 | 3E−168 | 75 | 143343_i_at |
| LocusLink: 29959 | FBgn0027497 | CG1098 | 479 | 2E−135 | 53 | 144182_at |
| LocusLink: 29988 | FBgn0034247 | CG6484 | 256 | 2.3E−68 | 34 | 142386_at |
| LocusLink: 3015 | FBgn0001197 | CG5499 | 190 | 2.2E−49 | 100 | 154022_at |
| LocusLink: 3654 | FBgn0010441 | CG5974 | 159 | 4.1E−39 | 34 | 142881_at |
| LocusLink: 3845 | FBgn0003205 | CG9375 | 266 | 4.5E−72 | 87 | 143317_at |
| LocusLink: 3988 | FBgn0032265 | CG18301 | 159 | 2.3E−39 | 29 | 146166_at |
| LocusLink: 4086 | FBgn0011648 | CG12399 | 662 | 0 | 74 | 143670_at |
| LocusLink: 4117 | FBgn0032164 | CG4588 | 430 | 2E−120 | 65 | 146112_at |
| LocusLink: 4125 | FBgn0027611 | CG6206 | 800 | 0 | 45 | 151851_at |
| LocusLink: 4285 | FBgn0033038 | CG7791 | 636 | 0 | 48 | 141715_at |
| LocusLink: 43 | FBgn0015568 | CG1031 | 208 | 6.6E−54 | 31 | 154652_at |
| LocusLink: 4547 | FBgn0032904 | CG9342 | 221 | 2.2E−57 | 25 | 141458_at |
| LocusLink: 4583 | FBgn0030561 | CG5228 | 367 | 2E−101 | 37 | 145061_at |
| LocusLink: 4640 | FBgn0010246 | CG9155 | 720 | 0 | 45 | 151960_at |
| LocusLink: 4677 | FBgn0028492 | CG10687 | 807 | 0 | 73 | 152066_at |
| LocusLink: 4848 | FBgn0017550 | CG2161 | 221 | 8E−58 | 54 | 153321_at |
| LocusLink: 5007 | FBgn0020626 | CG6708 | 549 | 3E−156 | 45 | 154587_at |
| LocusLink: 5066 | FBgn0033466 | CG12130 | 242 | 5.3E−64 | 33 | 154695_at |
| LocusLink: 5081 | FBgn0003145 | CG6716 | 318 | 8.7E−87 | 68 | 152870_at |
| LocusLink: 51116 | FBgn0031639 | CG2937 | 260 | 8.8E−70 | 61 | 145784_at |
| LocusLink: 51181 | FBgn0030968 | CG7322 | 192 | 2.1E−49 | 46 | 145332_at |
| LocusLink: 51200 | FBgn0033774 | CG12374 | 193 | 1.6E−49 | 30 | 147118_at |
| LocusLink: 51390 | FBgn0031245 | CG3625 | 173 | 1.1E−43 | 38 | 141831_at |
| LocusLink: 51533 | FBgn0031091 | CG9576 | 167 | 1.6E−41 | 35 | 155024_at |
| LocusLink: 5189 | FBgn0013563 | CG6760 | 313 | 2.9E−85 | 40 | 155032_at |
| LocusLink: 5225 | FBgn0032049 | CG13095 | 295 | 3.3E−80 | 43 | 152369_at |
| LocusLink: 5286 | FBgn0015278 | CG11621 | 719 | 0 | 36 | 142750_at |
| LocusLink: 5287 | FBgn0015278 | CG11621 | 719 | 0 | 39 | 142750_at |
| LocusLink: 5289 | FBgn0015277 | CG5373 | 520 | 1E−147 | 61 | 151517_at |
| LocusLink: 5331 | FBgn0004611 | CG4574 | 799 | 0 | 47 | 153733_at |
| LocusLink: 5428 | FBgn0004406 | CG8987 | 880 | 0 | 44 | 143008_at |
| LocusLink: 5439 | FBgn0032634 | CG6840 | 188 | 4E−49 | 78 | 146402_at |
| LocusLink: 54437 | FBgn0028679 | CG10913 | 589 | 5E−168 | 37 | 144243_at |
| LocusLink: 54933 | FBgn0030318 | CG1697 | 143 | 1.1E−34 | 34 | 141678_at |
| LocusLink: 5494 | FBgn0035425 | CG17746 | 214 | 5.7E−56 | 40 | 154425_at |
| LocusLink: 55349 | FBgn0001112 | CG1152 | 255 | 7.1E−68 | 34 | 143178_at |
| LocusLink: 55572 | FBgn0033093 | CG3270 | 374 | 6E−104 | 49 | 151882_at |
| LocusLink: 55632 | FBgn0005683 | CG5354 | 177 | 1.3E−44 | 33 | 153746_at |
| LocusLink: 5564 | FBgn0033383 | CG8057 | 255 | 1.8E−68 | 62 | 153026_at |
| LocusLink: 55677 | FBgn0030738 | CG9915 | 269 | 4.8E−72 | 43 | 145179_at |
| LocusLink: 55711 | FBgn0032055 | CG13091 | 220 | 1.5E−57 | 38 | 153064_at |
| LocusLink: 55920 | FBgn0031769 | CG9135 | 407 | 8E−114 | 48 | 154877_at |
| LocusLink: 5613 | FBgn0000489 | CG6117 | 441 | 5E−124 | 62 | 143130_at |
| LocusLink: 5645 | FBgn0032947 | CG17571 | 146 | 1.5E−35 | 37 | 146587_at |
| LocusLink: 5645 | FBgn0010425 | CG18681 | 144 | 5.6E−35 | 38 | 143624_at |
| LocusLink: 56997 | FBgn0030430 | CG4410 | 366 | 3E−101 | 51 | 151663_at |
| LocusLink: 57084 | FBgn0034394 | CG15096 | 144 | 4E−35 | 34 | 153069_at |
| LocusLink: 57122 | FBgn0027868 | CG6743 | 466 | 4E−131 | 33 | 144196_at |
| LocusLink: 57187 | FBgn0031390 | CG4263 | 355 | 6E−98 | 58 | 155165_at |
| LocusLink: 57508 | FBgn0030858 | CG8211 | 409 | 3E−114 | 43 | 153910_at |
| LocusLink: 57599 | FBgn0033607 | CG9062 | 699 | 0 | 56 | 147005_at |
| LocusLink: 5768 | FBgn0033814 | CG4670 | 263 | 2E−70 | 31 | 147135_at |
| LocusLink: 5770 | FBgn0003138 | CG9181 | 298 | 4.7E−81 | 49 | 141300_at |
| LocusLink: 5825 | FBgn0031069 | CG12703 | 719 | 0 | 59 | 145395_at |
| LocusLink: 58516 | FBgn0029738 | CG4068 | 147 | 1.6E−35 | 69 | 144501 _at |
| LocusLink: 5876 | FBgn0028970 | CG18627 | 415 | 2E−116 | 64 | 152961_at |
| LocusLink: 6262 | FBgn0011286 | CG10844 | 464 | 8E−131 | 58 | 143650_at |
| LocusLink: 6263 | FBgn0011286 | CG10844 | 465 | 4E−131 | 57 | 143650_at |
| LocusLink: 6342 | FBgn0032715 | CG17597 | 539 | 1E−153 | 63 | 141671_s_at |
| LocusLink: 6397 | FBgn0031814 | CG9528 | 580 | 1E−165 | 44 | 145899_at |
| LocusLink: 64137 | FBgn0031220 | CG4822 | 375 | 6E−104 | 37 | 143011_at |
| LocusLink: 64137 | FBgn0031516 | CG9663 | 240 | 3.2E−63 | 48 | 152940_at |
| LocusLink: 64172 | FBgn0031060 | CG14231 | 185 | 3E−47 | 37 | 154795_at |
| LocusLink: 6434 | FBgn0003742 | CG10128 | 137 | 7.1E−33 | 50 | 141588_at |
| LocusLink: 64426 | FBgn0031036 | CG14220 | 198 | 6E−51 | 42 | 145379_at |
| LocusLink: 64682 | FBgn0030639 | CG9198 | 251 | 1E−66 | 32 | 154789_at |
| LocusLink: 6515 | FBgn0033047 | CG7882 | 246 | 2.3E−65 | 35 | 146660_at |
| LocusLink: 6519 | FBgn0002570 | CG8696 | 347 | 1.1E−95 | 37 | 143242_at |
| LocusLink: 6519 | FBgn0033296 | CG11669 | 324 | 1.1E−88 | 36 | 146809_at |
| LocusLink: 6519 | FBgn0032382 | CG14935 | 362 | 3E−100 | 40 | 146257_at |
| LocusLink: 6519 | FBgn0033297 | CG8690 | 344 | 9.4E−95 | 38 | 146810_at |
| LocusLink: 6583 | FBgn0019952 | CG6331 | 196 | 2.1E−50 | 36 | 152843_at |
| LocusLink: 6583 | FBgn0033809 | CG4630 | 265 | 8E−71 | 32 | 153066_at |
| LocusLink: 6584 | FBgn0019952 | CG6331 | 196 | 1.6E−50 | 35 | 152843_at |
| LocusLink: 6584 | FBgn0033809 | CG4630 | 261 | 8.8E−70 | 32 | 153066_at |
| LocusLink: 6602 | FBgn0025463 | CG4303 | 587 | 8E−168 | 78 | 153648_at |
| LocusLink: 6652 | FBgn0024289 | CG1982 | 386 | 1E−107 | 55 | 141450_at |
| LocusLink: 6833 | FBgn0028675 | CG5772 | 205 | 3.2E−52 | 24 | 141744_at |
| LocusLink: 7694 | FBgn0034570 | CG10543 | 151 | 3.2E−36 | 34 | 147592_at |
| LocusLink: 79183 | FBgn0032783 | CG10237 | 151 | 6.4E−37 | 35 | 154963_at |
| LocusLink: 79258 | FBgn0027570 | CG9761 | 501 | 5E−142 | 37 | 152128_at |
| LocusLink: 79934 | FBgn0030430 | CG4410 | 372 | 4E−103 | 51 | 151663_at |
| LocusLink: 80013 | FBgn0030973 | CG7332 | 381 | 6E−106 | 42 | 154261_at |
| LocusLink: 80155 | FBgn0031020 | CG12202 | 627 | 8E−180 | 60 | 141774_at |
| LocusLink: 80221 | FBgn0031703 | CG12512 | 429 | 3E−120 | 39 | 145821_at |
| LocusLink: 8106 | FBgn0005648 | CG2163 | 211 | 3.9E−55 | 72 | 143566_i_at |
| LocusLink: 81492 | FBgn0034957 | CG3121 | 147 | 3.1E−35 | 29 | 152491_at |
| LocusLink: 81616 | FBgn0027348 | CG4501 | 520 | 2E−147 | 43 | 153318_at |
| LocusLink: 81796 | FBgn0034716 | CG3380 | 256 | 4.7E−68 | 28 | 142132_at |
| LocusLink: 829 | FBgn0034577 | CG10540 | 376 | 1E−104 | 64 | 152284_at |
| LocusLink: 830 | FBgn0034577 | CG10540 | 369 | 2E−102 | 61 | 152284_at |
| LocusLink: 8310 | FBgn0031813 | CG9527 | 510 | 2E−144 | 42 | 142194_at |
| LocusLink: 83544 | FBgn0028858 | CG10839 | 162 | 1.1E−40 | 52 | 144269_at |
| LocusLink: 836 | FBgn0028381 | CG14902 | 161 | 5.8E−40 | 37 | 144201_at |
| LocusLink: 840 | FBgn0028381 | CG14902 | 167 | 1.1E−41 | 32 | 144201_at |
| LocusLink: 84188 | FBgn0033464 | CG1441 | 316 | 2.1E−86 | 34 | 152521_at |
| LocusLink: 84191 | FBgn0034543 | CG10404 | 156 | 1.1E−38 | 57 | 147569_at |
| LocusLink: 8424 | FBgn0030575 | CG5321 | 256 | 2.6E−68 | 39 | 145074_at |
| LocusLink: 84312 | FBgn0030434 | CG4400 | 156 | 1.6E−38 | 42 | 144972_at |
| LocusLink: 84466 | FBgn0027594 | CG2086 | 280 | 2E−75 | 37 | 151918_s_at |
| LocusLink: 84947 | FBgn0032699 | CG10383 | 154 | 1.4E−37 | 37 | 152901_at |
| LocusLink: 85365 | FBgn0035401 | CG1291 | 398 | 4E−111 | 50 | 153189_at |
| LocusLink: 85415 | FBgn0026374 | CG8497 | 317 | 1.2E−86 | 37 | 144124_at |
| LocusLink: 85465 | FBgn0031948 | CG7149 | 217 | 7.4E−57 | 46 | 152382_at |
| LocusLink: 8708 | FBgn0031988 | CG8668 | 150 | 2.3E−36 | 33 | 154502_at |
| LocusLink: 8882 | FBgn0030096 | CG9060 | 407 | 6E−114 | 48 | 142846_at |
| LocusLink: 89978 | FBgn0030336 | CG1578 | 318 | 6.3E−87 | 60 | 154432_at |
| LocusLink: 4799 | FBgn0001978 | CG3647 | 224 | 0 | 41 | 151662_s_at |
| LocusLink: 9060 | FBgn0020389 | CG8363 | 786 | 0 | 62 | 154327_at |
| LocusLink: 9061 | FBgn0020389 | CG8363 | 788 | 0 | 62 | 154327_at |
| LocusLink: 9079 | FBgn0013764 | CG3924 | 430 | 1E−120 | 62 | 154317_at |
| LocusLink: 9100 | FBgn0035174 | CG13903 | 299 | 5.6E−81 | 40 | 142762_at |
| LocusLink: 9150 | FBgn0035026 | CG12252 | 382 | 7E−106 | 35 | 141451_at |
| LocusLink: 94233 | FBgn0014019 | CG5279 | 187 | 1.3E−47 | 33 | 153085_at |
| LocusLink: 94233 | FBgn0019940 | CG5192 | 194 | 7.9E−50 | 38 | 143905_at |
| LocusLink: 9444 | FBgn0017397 | CG10293 | 277 | 1E−74 | 50 | 143885_at |
| LocusLink: 9535 | FBgn0028894 | CG5869 | 150 | 3.2E−37 | 52 | 141567_at |
| LocusLink: 9619 | FBgn0031220 | CG4822 | 378 | 6E−105 | 37 | 143011_at |
| LocusLink: 9619 | FBgn0031516 | CG9663 | 252 | 8.1E−67 | 42 | 152940_at |
| LocusLink: 9619 | FBgn0003996 | CG2759 | 280 | 1.8E−75 | 32 | 143410_at |
| LocusLink: 9632 | FBgn0031408 | CG10882 | 800 | 0 | 52 | 141363_at |
| LocusLink: 9690 | FBgn0034989 | CG3356 | 673 | 0 | 36 | 141336_at |
| LocusLink: 9717 | FBgn0031814 | CG9528 | 570 | 8E−163 | 43 | 145899_at |
| LocusLink: 9785 | FBgn0030550 | CG1405 | 1354 | 0 | 58 | 154291_at |
| LocusLink: 9924 | FBgn0033352 | CG8232 | 726 | 0 | 39 | 154804_at |
| LocusLink: 994 | FBgn0003525 | CG1395 | 193 | 1.9E−49 | 38 | 141264_at |

Human genes are listed by Locus identification number (www.ncbi.nlm.nih.gov).
Drosophila genes are listed by Flybase gene identification number (FBgn) and its synonymous CG identification number (CG) (www.flybase.org).
The homology between the human and the Drosophila genes are demonstrated by the blast score, blast probability and percentage of protein sequence homology based on BLASP.

### Example 5

Additional Transgene Expression and Total RNA Isolation

Two additional genes in the insulin signaling pathway, dS6K and dPDK1. See Alessi et al.

3-phosphoinositide-dependent protein kinase-1 (PDK1): structural and functional homology with the Drosophila DSTPK61 kinase, Curr Biol, Vol. 7, No. 10, pp. 776-789 (1997) are overexpressed in flies using the UAS-dS6K transgene. See Stewart and Barcelo, Genesis, Vol. 34, pp. 83-85 (2002) and EP(3)3553 (http://flybase.bio.indiana.edu/). UAS-dPDK1 transgenes, and total RNAs were isolated, by the same method as described in the “EXAMPLES” section, above, and illustrated in FIG. 1.

### Example 6

Alternative Method of Microarray Data Analysis

Experimental Flies and Control Flies

Male progeny flies carrying both hsp-Gal4 and UAS-transgene are served as the experimental flies for each overexpression experiment. Male flies carrying only hsp-Gal4 are served as the control flies for each overexpression experiment.

Male flies carrying both hsp-Gal4 and UAS-GFP are used to filter out genes whose transcription is affected by the induction of protein unrelated to ISP.

Identification of DEGs

The GeneChip™ Drosophila genome array used in this study contains 13,966 probe sets representing approximately 13,282 genes (many genes are represented by more than one probe set). The hybridization intensity data is calculated from the images generated by the Gene Array scanner (Affymetrix), using the Affymetrix Microarray Suite (MAS) 5.0. Microarrays are normalized by Affymetrix default settings in a way that the trimmed mean is set to a constant value and that the resulting scale factor is applied to all expression values of each chip. The trimmed mean is the average expression value after removing the 2% lowest and 2% highest observations. As the constant target value an average expression value of 150 is used. For each overexpression experiment, the identification of DEGs is performed on the signals obtained from the 4 samples of the experimental group versus the 4 samples of the control group using an R package [see Schwender (2003)] implementing the SAM algorithm as described in Tusher et al. (2001). The number of random experiment-label permutations is set to 100. The factor “s0” is computed as the minimum co-efficient of variation of the relative distance as a function of the gene specific scatter and turns out to be 0 in most cases. Affymetrix control probe sets and probe sets with “absent” calls in all 8 samples related to one experiment are discarded in the SAM analysis of the respective experiment. The selection criteria of the DEGs for each overexpression experiment are shown in Table 14. Briefly, the initial cut-off criteria is set as SAM q-value of ≦3% and fold change of ≧1.5. Furthermore, to limit the total number of DEGs for the follow-up analysis, the upper bound of the percentage of probe sets passing the SAM q-value and fold-change cutoffs in all the probe sets passing Affy MAS5 absent-call filtering is set at <10% for each experiment. SAM q-values are adjusted to meet this criterion for some experiments. Final numbers of differentially-expressed probe sets from each overexpression experiment are obtained by further filtering-out probe sets affected by GFP overexpression using the respective actually used cut-off criteria (see Table 14).

Gene Ontology Analysis

Every probe set on the GeneChip™ Drosophila genome array is annotated by integrating the information on the gene ontology (GO) web site (http://www.geneontology.org) with the information available from NetAFFX. See Liu et al. (2003). Each probe set on the chip is associated with its current gene entry (FBgn number). Next, each gene is associated to its available GO annotations for biological processes. The GO tree structure is restored and the number of genes that are annotated as belonging to a particular GO term and its child GO terms are annotated. Next, we calculate binomial probabilities to determine whether there is a strong association between a particular GO term and differential gene expression by the overexpression of an ISP component. For example, when overexpressing dPTEN, there are 493 differentially-expressed probe sets identified, corresponding to 488 genes. This is approximately 3.7% of the genes represented on the chip. We then compare the number of genes associated at and below a specific GO term from the differentially-expressed genes in response to dPTEN overexpression with the analogous number for the entire chip. If the DEGs from dPTEN overexpression experiment comprise 3.7% of the whole genome, then under the null hypothesis of no relationship between differential gene expression regulated by dPTEN overexpression and biological process, we expect (for each GO term) 3.7/100 as many genes from the DEGs in response to dPTEN overexpression as there are on the entire chip. For example, for the GO term protein metabolism, 671 genes are found on the chip, and 52 are represented in the DEGs from dPTEN overexpression experiment (see Table 16). The p-value associated with the null hypothesis of no association is obtained from binomial distribution with 671 tries, 0.037 probability of success, and >52 successes (p=4.84E−08). After multiple test correction by Benjamini and Hochberg false discovery rate [see Benjamini and Hochberg (1995)], we obtain a p-value of 7.00E−06. Thus, in this case, there is a significant association between protein metabolism and differential gene expression by the overexpression of dPTEN.

Identification of ISP-Regulated Genes

In this alternative analysis, a total of 631, 384, 493, 662, 710, 540 and 837 probe sets are found to be differentially-regulated following dAkt1, Dp110^{CAAX}, dPTEN, Dp110^{D954A}, dfoxo, dPDK1 and dS6K overexpression, respectively (see Table 15). These correspond to approximately 4.52%, 2.75%, 3.53%, 4.74%, 5.08%, 3.87% and 5.99% of the probe sets represented on the array, respectively. The complete DEG lists from different experiments are shown in Tables 18-25.

Biological Process Classification of DEGs

Molecular genetic studies have demonstrated that ISP in Drosophila regulates growth, cell proliferation, metabolism and aging. We were also interested in finding out what biological processes were affected by the overexpression of the known ISP components in Drosophila at transcription level. We used the annotation project directed by the GO to functionally classify these DEGs.

Although rapidly evolving, the GO contains information about biological processes on approximately 19% (2,477/13,282) of the genes represented on our array according to this analysis. Several biological processes are significantly over-represented in the sets of DEGs identified from different overexpression experiments (see details in Supplementary). A summary of biological process classification of the ISP-regulated DEGs is presented in Table 17.

Metabolism

ISP has been shown to regulate cellular metabolism. Therefore it is not surprising to see that certain metabolic processes are overrepresented by overexpression of ISP components. For example, there are significant associations between protein biosynthesis and metabolism and differential gene regulation by dPTEN and Dp110^{D954A}. There are 26 and 31 ribosomal or ribosomal-like proteins differentially-regulated by dPTEN and Dp110^{D954A}, respectively, 22 of them regulated by both genes. These genes are generally down-regulated by dPTEN, Dp110^{D954A} and dfoxo, and relatively no change by dAkt1, Dp110^{CAAX}, dPDK1 and dS6K. Moreover, two genes [Su(var)3-9 and eRF1] involved in translational initiation and termination, respectively, are differentially-regulated by both dPTEN and Dp110^{D954A}, respectively. Three other genes (Paip2, elF3-S8 and elF3-S9) involved in negative regulation of translation and translational initiation are also differentially-regulated by Dp110^{D954A}. Taken together, it clearly shows that protein biosynthesis and the general translational machinery are regulated by ISP components.

Also interestingly, several kinase-encoding genes involved in protein amino acid phosphorylation are differentially-regulated by either dPTEN or Dp110^{D954A}, or by both genes. For example, SAK, which is up-regulated by Dp110^{D954A}, encodes a protein serine/threonine kinase that is required for appropriate exit from mitosis. See Hudson et al. (2001). MAPk-Ak2, which is up-regulated by Dp110^{D954A}, encodes a MAP kinase activated, protein serine/threonine kinase that phosphorylates small heat-shock proteins. See Rouse et al. (1994); and Larochelle and Suter (1995). CaMKII, which is also up-regulated by Dp110^{D954A}, encodes a calcium/calmodulin-dependent, protein serine/threonine kinase that is one of the major protein kinases coordinating cellular responses to neurotransmitters and hormones. See Ohsako et al. (1993); and Griffith et al. (1993). Wee, which is up-regulated by both dPTEN and Dp110^{D954A}, encodes a protein tyrosine kinase that is a Cdc2 inhibitory kinase required for preventing premature activation of the mitotic program. See Campbell et al. (1995). Interestingly, generally speaking, these genes are up-regulated by dPTEN, Dp110^{D954A} and dfoxo, and relatively no change or slightly down-regulated by dAkt1, Dp110^{CAAX}, dPDK1 and dS6K.

PI3K-PKB-Forkhead signaling has been shown to protect quiescent cells from oxidative stress in mammalian systems. See Kops et al. (2002). Reactive oxygen species are a primary cause of cellular damage that leads to cell death. PKB-regulated Forkhead transcription factor FOXO3a has been shown to be able to protect quiescent cells from oxidative stress by directly increasing their quantities of manganese superoxide dismutase (MnSOD, encoded by SOD2) mRNA and protein. Consistent with this observation from mammalian study, the fly homolog of human SOD2 is also up-regulated by dfoxo overexpression, and the biological process “superoxide metabolism” is over-represented (multiple test adjusted p=0.077). Also interestingly, electron transport process is over-represented by dfoxo overexpression, with CoVa, CG4769, Cyt-c-p (encode a cytochrome c oxidase, a Cytochrome_C1-like electron transporter and an electron transporter, respectively) up-regulated whereas Cyt-b5 and Trxr-1 (encode an electron transporter and a thioredoxin reductase) down-regulated.

IMP metabolism/biosynthesis was found to be significantly associated with differential gene expression by dS6K overexpression. Three genes ade2, ade3 and Prat, encoding a phosphoribosylformylglycinamidine synthase, a phosphoribosylamine-glycine ligase, and a amidophosphoribosyltransferase, respectively, are up-regulated by dS6K overexpression.

TABLE 14
Overview of the Filter and Cut-Offs Used to Identify Differentially-Expressed
Probe Sets for Each Overexpression Experiment

| | Probe Sets Passing | Cut-Off Criteria | | | |
|---|---|---|---|---|---|
| Overexpression | Affy MAS5 Absent- | SAM | | | Probe Sets Passing |
| Experiment | Call Filtering^{a} | q-value | Fold | %^{b} | Cut-Offs^{c} |
| dPDK1 | 9568 | ≦3% | ≧1.5 | 5.6 | 540 |
| dS6K | 9548 | ≦3% | ≧1.5 | 8.8 | 837 |
| Dp110^{CAAX} | 7166 | ≦3% | ≧1.5 | 5.4 | 384 |
| dAkt1 | 7275 | ≦3% | ≧1.5 | 8.7 | 631 |
| Dp110^{D954A} | 7926 | ≦2% | ≧1.5 | 8.4 | 662 |
| dPTEN | 7412 | ≦1.5% | ≧1.5 | 6.7 | 493 |
| dfoxo | 7648 | ≦0.59% | ≧1.5 | 9.3 | 710 |
| ^{a}Probe sets with MAS5 absent calls in all samples of each experiment were discarded. | | | | | |
| ^{b}Represents percentage of probe sets passing the SAM q-value and fold-change cut-offs in all the probe sets passing Affy MAS5 absent-call filtering for each experiment. The SAM q-value and fold-change cutoffs were | | | | | |
| # initially set at 3% and 1.5, respectively. Furthermore, the upper bound of the percentage of probe sets passing the SAM q-value and fold-change cut-offs in all the probe sets passing Affy MAS5 absent-call filtering was set | | | | | |
| # at 10% for each experiment. SAM q-values were adjusted to meet this criterion. | | | | | |

^{c}Final numbers of differentially-expressed probe sets from each overexpression experiment were obtained by filtering out probe sets affected by GFP overexpression using the respective actually used cut-off criteria.

TABLE 15
Overview of the Numbers of Probe Sets Differentially-Expressed
Following Overexpression of Major ISP Components

| Overexpression | | Up- | Down- |
|---|---|---|---|
| Experiment | Total | Regulated | Regulated |
| dAkt1 | 631 | 243 | 388 |
| Dp110^{CAAX} | 384 | 187 | 197 |
| dfoxo | 710 | 321 | 389 |
| dPTEN | 493 | 192 | 301 |
| Dp110^{D954A} | 662 | 275 | 387 |
| dPDK1 | 540 | 292 | 248 |
| dS6K | 837 | 482 | 355 |

TABLE 16
Numbers of Overlapped Differentially-Regulated Genes Between
Different Overexpression Experiments

| | Dp110^{CAAX} | (384) | dfoxo | (710) | dPTEN | (493) | Dp110^{D954A} | (662) | dPDK1 | (540) | dS6K | (837) |
|---|---|---|---|---|---|---|---|---|---|---|---|---|
| | ↑ | ↓ | ↑ | ↓ | ↑ | ↓ | ↑ | ↓ | ↑ | ↓ | ↑ | ↓ |
| | (187) | (197) | (321) | (389) | (192) | (301) | (275) | (387) | (292) | (248) | (482) | (355) |
| dAkt1 (631) | | | | | | | | | | | | |
| ↑ (243) | 0 | | 10 | | 6 | | 10 | | 15 | 14 | 4 | 51 |
| ↓ (388) | 1 | 105 | 9 | 128 | 4 | 102 | 4 | 98 | 54 | 12 | 104 | 7 |
| Dp110^{CAAX} (384) | | | | | | | | | | | | |
| ↑ (187) | | | 27 | | 6 | | 15 | | 8 | 25 | 7 | 43 |
| ↓ (197) | | | 9 | 82 | 1 | 51 | 1 | 54 | 55 | 6 | 80 | 9 |
| dfoxo (710) | | | | | | | | | | | | |
| ↑ (321) | | | | | 1 | | 2 | | 20 | 6 | 34 | 10 |
| ↓ (389) | | | | | 0 | 182 | 1 | 169 | 58 | 18 | 78 | 30 |
| dPTEN (493) | | | | | | | | | | | | |
| ↑ (192) | | | | | | | 1 | | 8 | 15 | 9 | 7 |
| ↓ (301) | | | | | | | 0 | 192 | 30 | 13 | 50 | 9 |
| Dp110^{D954A} (662) | | | | | | | | | | | | |
| ↑ (275) | | | | | | | | | 13 | 13 | 23 | 5 |
| ↓ (387) | | | | | | | | | 25 | 29 | 41 | 30 |
| dPDK1 (540) | | | | | | | | | | | | |
| ↑ (292) | | | | | | | | | | | 135 | 5 |
| ↓ (248) | | | | | | | | | | | 17 | 60 |

Overview of the numbers of overlapped genes that were differentially-expressed following overexpression of ISP components.
↑ = up-regulation following overexpression
↓ = down-regulation following overexpression
Numbers in parentheses represent the number of differentially-regulated genes in each category.

TABLE 17
Biological Process Classification of Genes Significantly Differentially-Regulated
Following Overexpression of ISP Components Using GO

| Process Ontology | GO ID | Chip | dPTEN | Dp110^{D954A} | dS6K | dfoxo | Dp110^{CAAX} | dAKT1 | dPDK1 |
|---|---|---|---|---|---|---|---|---|---|
| Metabolism | GO: 0008152 | 1347 | 75** | 95** | | | | | |
| Biosynthesis | GO: 0009058 | 305 | 29** | 42** | | | | | |
| Macromolecule | GO: 0009059 | 213 | 29** | 37** | | | | | |
| biosynthesis | | | | | | | | | |
| Protein biosynthesis | GO: 0006412 | 213 | 29** | 37** | | | | | |
| Protein metabolism | GO: 0019538 | 671 | 52** | 64** | | | | | |
| Hormone metabolism | GO: 0042445 | 9 | 3* | | | | | | |
| Hormone catabolism | GO: 0042447 | 2 | 2* | | | | | | |
| Lipid catabolism | GO: 0016042 | 2 | 2* | | | | | | |
| Isoprenoid catabolism | GO: 0008300 | 2 | 2* | | | | | | |
| Polyisoprenoid | GO: 0016097 | 2 | 2* | | | | | | |
| catabolism | | | | | | | | | |
| Terpenoid catabolism | GO: 0016115 | 2 | 2* | | | | | | |
| Sesquiterpenoid | GO: 0016107 | 2 | 2* | | | | | | |
| catabolism | | | | | | | | | |
| Juvenile hormone | GO: 0006719 | 2 | 2* | | | | | | |
| catabolism | | | | | | | | | |
| Polyisoprenoid | GO: 0016096 | 2 | 2* | | | | | | |
| metabolism | | | | | | | | | |
| Terpenoid metabolism | GO: 0006721 | 2 | 2* | | | | | | |
| Sesquiterpenoid | GO: 0006714 | 2 | 2* | | | | | | |
| metabolism | | | | | | | | | |
| Juvenile hormone | GO: 0006716 | 2 | 2* | | | | | | |
| metabolism | | | | | | | | | |
| Terpene metabolism | GO: 0042214 | 2 | 2* | | | | | | |
| Terpene catabolism | GO: 0046247 | 2 | 2* | | | | | | |
| Nucleobase, nucleoside, | | | | | | | | | |
| nucleotide and nucleic | | | | | | | | | |
| acid metabolism | | | | | | | | | |
| Nucleotide metabolism | | | | | | | | | |
| Nucleoside | GO: 0009123 | 5 | | | 3* | | | | |
| monophosphate | | | | | | | | | |
| metabolism | | | | | | | | | |
| Nucleoside | GO: 0009124 | 5 | | | 3* | | | | |
| monophosphate | | | | | | | | | |
| biosynthesis | | | | | | | | | |
| Purine nucleoside | GO: 0009127 | 5 | | | 3* | | | | |
| monophosphate | | | | | | | | | |
| biosynthesis | | | | | | | | | |
| Purine nucleoside | GO: 0009126 | 5 | | | 3* | | | | |
| monophosphate | | | | | | | | | |
| metabolism | | | | | | | | | |
| Ribonucleoside | GO: 0009161 | 5 | | | 3* | | | | |
| monophosphate | | | | | | | | | |
| metabolism | | | | | | | | | |
| Ribonucleoside | GO: 0009156 | 5 | | | 3* | | | | |
| monophosphate | | | | | | | | | |
| biosynthesis | | | | | | | | | |
| Purine ribonucleoside | GO: 0009167 | 5 | | | 3* | | | | |
| monophosphate | | | | | | | | | |
| metabolism | | | | | | | | | |
| Purine ribonucleoside | GO: 0009168 | 5 | | | 3* | | | | |
| monophosphate | | | | | | | | | |
| biosynthesis | | | | | | | | | |
| IMP metabolism | GO: 0046040 | 4 | | | 3* | | | | |
| IMP biosynthesis | GO: 0006188 | 4 | | | 3* | | | | |
| De novo IMP biosynthesis | GO: 0006189 | 4 | | | 3* | | | | |
| Electron transport | GO: 0006118 | 16 | | | | 5* | | | |
| Cell growth and/or | | | | | | | | | |
| maintenance | | | | | | | | | |
| Cell growth | | | | | | | | | |
| Regulation of cell growth | | | | | | | | | |
| Positive regulation of | GO: 0030307 | 7 | 3* | | | | | | |
| cell growth | | | | | | | | | |
| Transport | | | | | | | | | |
| Lipid transport | GO: 0006869 | 2 | 2* | | | | | | |
| Hydrogen transport | GO: 0006818 | 14 | | | | 5* | | | |
| Response to external | GO: 0009605 | 272 | | | 31* | 30** | 17* | 34** | |
| stimulus | | | | | | | | | |
| Response to abiotic | | | | | | | | | |
| stimulus | | | | | | | | | |
| Response to chemical | | | | | | | | | |
| substance | | | | | | | | | |
| Response to toxin | GO: 0009636 | 14 | 4* | | | | | | |
| Response to insecticide | GO: 0017085 | 12 | 4* | | | | | | |
| Response to temperature | GO: 0009266 | 22 | 5* | 7** | | 6* | | 9** | |
| Response to heat | GO: 0009408 | 21 | 5* | 7** | | | | 8** | |
| Response to biotic | GO: 0009607 | 116 | | | 17* | 18** | 13* | 19** | |
| stimulus | | | | | | | | | |
| Defense response | GO: 0006952 | 101 | | | 17* | 17** | 12* | 17** | |
| Immune response | GO: 0006955 | 54 | | | 11* | 11** | 7* | 10* | |
| Humoral immune response | GO: 0006959 | 45 | | | 11* | 10** | 7* | 8* | |
| Antimicrobial humoral | GO: 0019730 | 43 | | | 10* | 9* | 7* | | |
| response | | | | | | | | | |
| Humoral defense | GO: 0016065 | 36 | | | 10* | 9** | 6* | 7* | |
| mechanism (sensu | | | | | | | | | |
| Invertebrata) | | | | | | | | | |
| Antimicrobial humoral | GO: 0006960 | 34 | | | 9* | 8* | 6* | | |
| response (sensu | | | | | | | | | |
| Invertebrata) | | | | | | | | | |
| Response to | GO: 0009613 | 49 | | | 12* | 11** | 7* | 9* | |
| pest/pathogen/parasite | | | | | | | | | |
| Response to stress | GO: 0006950 | 96 | | 15** | 17* | 18** | 9* | 20** | |
| Homeostasis | GO: 0042592 | 15 | | | | | | | 6** |
| Cell homeostasis | GO: 0019725 | 15 | | | | | | | 6** |
| Development | | | | | | | | | |
| Oogenesis (sensu Insecta) | GO: 0009993 | 113 | | 15* | | | | | |

GO IDs and terms are provided for the pathways that show significant over-representation in the sets of DEGs identified from different overexpression experiments.
Data comprise the number of genes in each GO category present on the entire chip and the number present in the set of DEGs from each overexpression experiment.
**= a higher significant association between the functional group and overexpression of an insulin pathway component (p < 0.05, multiple test correction by Benjamini and Hochberg false discovery rate).
*= a significant association between the functional group and overexpression of an insulin pathway component (p < 0.005, multiple test correction by Benjamini and Hochberg false discovery rate).

TABLE 18
The Top 20 Down-Regulated and Top 20 Up-Regulated Genes in Response to dAKT1 Overexpression Ranked by Fold Change

| | SAM | Fold | | Gene | | | |
|---|---|---|---|---|---|---|---|
| Probe Set | q-value | Change | FBgn | Symbol | EC Number | Biological Process (GO) | Molecular Function (GO) |
| 152766_at | 0.011 | 47.8 | FBgn0002563 | Lsp1&bgr; | — | | nutrient reservoir activity |
| 146819_at | 0.015 | 20.8 | FBgn0033307 | CG14752 | — | | |
| 143239_at | 0.010 | 19.4 | FBgn0002565 | Lsp2 | — | | nutrient reservoir activity |
| 141264_at | 0.020 | 11.9 | FBgn0003525 | stg | EC: 3.1.3.48 | tracheal cell fate | protein tyrosine |
| | | | | | | determination | phosphatase activity |
| | | | | | | (sensu Insecta) | |
| 146958_at | 0.021 | 11.4 | FBgn0033541 | CG12934 | — | | |
| 142220_at | 0.017 | 9.8 | FBgn0033367 | CG8193 | EC: 1.14.18.1 | defense response | monophenol |
| | | | | | | | monooxygenase activity |
| 152245_at | 0.011 | 9.3 | FBgn0033857 | CG13335 | — | | |
| 152078_at | 0.011 | 9.1 | FBgn0031824 | CG9547 | EC: 1.3.99.7 | | acyl-CoA dehydrogenase |
| | | | | | | | activity |
| 142733_at | 0.013 | 8.1 | FBgn0036948 | CG7298 | — | | chitin binding activity |
| 147430_at | 0.012 | 7.8 | FBgn0034328 | CG15066 | — | | |
| 149371_at | 0.013 | 7.7 | FBgn0037405 | CG1077 | — | | |
| 152687_at | 0.011 | 6.5 | FBgn0030688 | CG8952 | EC: 3.4.21 | | serine-type endopeptidase |
| | | | | | | | activity |
| 143112_at | 0.030 | 6.2 | FBgn0000360 | Cp38 | — | insect chorion | structural constituent of |
| | | | | | | formation | chorion (sensu Insecta) |
| 151927_at | 0.019 | 6.1 | FBgn0036619 | CG4784 | — | | |
| 141389_at | 0.024 | 6.1 | FBgn0034582 | CG10531 | EC: 3.2.1.14 | | hydrolase activity |
| 145820_at | 0.027 | 6.0 | FBgn0031701 | TotM | — | humoral defense | |
| | | | | | | mechanism (sensu | |
| | | | | | | Invertebrata) | |
| 146096_at | 0.027 | 5.7 | FBgn0032132 | CG4382 | EC: 3.1.1.1 | | carboxylic ester hydrolase |
| | | | | | | | activity |
| 149304_at | 0.015 | 5.7 | FBgn0037289 | CG2016 | — | | |
| 142217_at | 0.014 | 5.5 | FBgn0011276 | HLH3B | — | | transcription factor activity |
| 152747_at | 0.029 | 5.1 | FBgn0011828 | Pxn | EC: 1.11.1.7 | | peroxidase activity |
| 141292_at | 0.016 | 5.0 | FBgn0037370 | CG1236 | EC: 1.1.1.95 | | phosphoglycerate |
| | | | | | | | dehydrogenase activity |
| 147118_at | 0.011 | −4.6 | FBgn0033774 | CG12374 | EC: 3.4.17.1 | | carboxypeptidase A activity |
| 143295_at | 0.011 | −4.8 | FBgn0003046 | Pcp | — | | structural constituent of |
| | | | | | | | pupal cuticle (sensu |
| | | | | | | | Insecta) activity |
| 150937_at | 0.011 | −4.8 | FBgn0039840 | CG11340 | — | | extracellular ligand-gated |
| | | | | | | | ion channel activity |
| 148963_at | 0.012 | −5.1 | FBgn0036737 | CG6298 | EC: 3.4.21.1 | | trypsin activity |
| 154976_at | 0.016 | −5.2 | FBgn0004901 | Prat | EC: 2.4.2.14 | metabolism | amidophosphoribosyl- |
| | | | | | | | transferase activity |
| 152313_at | 0.025 | −5.3 | FBgn0015714 | Cyp6a17 | EC: 1.14.14.1 | electron transport | cytochrome P450 activity |
| 149767_at | 0.011 | −5.4 | FBgn0038033 | CG10097 | — | | |
| 143835_at | 0.010 | −5.4 | FBgn0015657 | DnaJ-1 | — | | chaperone activity |
| 151932_at | 0.010 | −5.6 | FBgn0000406 | Cyt-b5-r | — | electron transport | electron transporter activity |
| 143468_at | 0.011 | −5.9 | FBgn0004429 | LysP | EC: 3.2.1.17 | antimicrobial | lysozyme activity |
| | | | | | | humoral response | |
| | | | | | | (sensu | |
| | | | | | | Invertebrata) | |
| 141450_at | 0.010 | −7.9 | FBgn0024289 | Sodh-1 | EC: 1.1.1.14 | | L-iditol 2-dehydrogenase |
| | | | | | | | activity |
| 150574_at | 0.010 | −8.6 | FBgn0039309 | CG11891 | — | | |
| 145061_at | 0.017 | −8.8 | FBgn0030561 | CG5228 | — | | |
| 146257_at | 0.011 | −8.9 | FBgn0032382 | CG14935 | EC: 3.2.1.20 | | alpha-amylase activity |
| 151781_at | 0.016 | −9.1 | FBgn0039342 | CG5107 | — | | |
| 143038_at | 0.010 | −9.6 | FBgn0037207 | Mes2 | — | | |
| 151967_at | 0.011 | −9.8 | FBgn0032287 | CG6415 | EC: 2.1.2.10 | | aminomethyltransferase |
| | | | | | | | activity |
| 150206_at | 0.010 | −11.2 | FBgn0038718 | CG17752 | — | | transporter activity |
| 150699_at | 0.010 | −14.7 | FBgn0039471 | CG6295 | EC: 3.1.1.3 | | enzyme activity |
| 143789_at | 0.010 | −18.9 | FBgn0015279 | Pi3K92E | EC: 2.7.1.137 | phosphorylation | phosphatidylinositol 3- |
| | | | | | | | kinase activity |

TABLE 19
The Top 20 Down-Regulated and Top 20 Up-Regulated Genes in Response
to Dp110^{CAAX} Overexpression Ranked by Fold Change

| | SAM | Fold | | Gene | | Biological | |
|---|---|---|---|---|---|---|---|
| Probe Set | q-value | Change | FBgn | Symbol | EC Number | Process (GO) | Molecular Function (GO) |
| 152766_at | 0.022 | 12.6 | FBgn0002563 | Lsp1&bgr; | — | | nutrient reservoir activity |
| 144058_at | 0.020 | 11.9 | FBgn0025390 | EG: 56G7.1 | — | | chitin binding activity |
| 146819_at | 0.024 | 9.6 | FBgn0033307 | CG14752 | — | | |
| 143470_at | 0.024 | 9.2 | FBgn0004431 | LysX | EC: 3.2.1.17 | antimicrobial | lysozyme activity |
| | | | | | | humoral response | |
| | | | | | | (sensu | |
| | | | | | | Invertebrata) | |
| 143239_at | 0.021 | 7.8 | FBgn0002565 | Lsp2 | — | | nutrient reservoir activity |
| 149371_at | 0.024 | 6.9 | FBgn0037405 | CG1077 | — | | |
| 149304_at | 0.023 | 6.6 | FBgn0037289 | CG2016 | — | | |
| 149808_at | 0.021 | 6.6 | FBgn0038095 | Cyp304a1 | — | electron transport | cytochrome P450 activity |
| 146141_at | 0.027 | 6.3 | FBgn0032221 | CG5375 | — | | |
| 143112_at | 0.029 | 6.2 | FBgn0000360 | Cp38 | — | insect chorion | structural constituent of |
| | | | | | | formation | chorion (sensu Insecta) |
| 142220_at | 0.022 | 5.6 | FBgn0033367 | CG8193 | EC: 1.14.18.1 | defense response | monophenol |
| | | | | | | | monooxygenase activity |
| 152078_at | 0.028 | 5.4 | FBgn0031824 | CG9547 | EC: 1.3.99.7 | | acyl-CoA dehydrogenase |
| | | | | | | | activity |
| 154291_at | 0.023 | 5.1 | FBgn0030550 | CG1405 | — | | |
| 141331_at | 0.020 | 4.9 | FBgn0033137 | Tsp42Ep | — | | |
| 152687_at | 0.021 | 4.8 | FBgn0030688 | CG8952 | EC: 3.4.21 | | serine-type |
| | | | | | | | endopeptidase activity |
| 144238_at | 0.025 | 4.7 | FBgn0028573 | prc | — | heart development | |
| 141389_at | 0.020 | 4.6 | FBgn0034582 | CG10531 | EC: 3.2.1.14 | | hydrolase activity |
| 146780_at | 0.024 | 4.6 | FBgn0033242 | CG2916 | — | | |
| 150760_at | 0.020 | 4.5 | FBgn0039564 | CG5527 | — | | endothelin-converting |
| | | | | | | | enzyme activity |
| 141292_at | 0.028 | 4.4 | FBgn0037370 | CG1236 | EC: 1.1.1.95 | | phosphoglycerate |
| | | | | | | | dehydrogenase activity |
| 148400_at | 0.024 | 4.3 | FBgn0035886 | CG7118 | EC: 3.4.21 | | trypsin activity |
| 150702_at | 0.024 | −4.3 | FBgn0039474 | CG6283 | EC: 3.1.1.3 | | enzyme activity |
| 152351_at | 0.022 | −4.3 | FBgn0035950 | CG5288 | EC: 2.7.1.6 | | galactokinase activity |
| 143385_at | 0.021 | −4.3 | FBgn0003863 | &agr; Try | EC: 3.4.21.4 | proteolysis and | trypsin activity |
| | | | | | | peptidolysis | |
| 149914_at | 0.021 | −4.4 | FBgn0038257 | smp-30 | — | | |
| 151091_at | 0.020 | −4.5 | FBgn0040606 | CG6503 | — | | |
| 150031_at | 0.022 | −4.6 | FBgn0038450 | CG17560 | — | | |
| 151797_at | 0.025 | −4.8 | FBgn0032820 | fbp | EC: 3.1.3.11 | | fructose-bisphosphatase |
| | | | | | | | activity |
| 150699_at | 0.021 | −4.9 | FBgn0039471 | CG6295 | EC: 3.1.1.3 | | enzyme activity |
| 143468_at | 0.021 | −5.0 | FBgn0004429 | LysP | EC: 3.2.1.17 | antimicrobial | lysozyme activity |
| | | | | | | humoral response | |
| | | | | | | (sensu | |
| | | | | | | Invertebrata) | |
| 150403_at | 0.022 | −5.0 | FBgn0039030 | CG6660 | — | | |
| 151490_s_at | 0.021 | −5.3 | GH04896.3 | CG12138 | — | | |
| | | | prime-hit | | | | |
| 153064_at | 0.021 | −5.5 | FBgn0032055 | CG13091 | — | | |
| 148253_at | 0.022 | −5.7 | FBgn0035664 | CG6467 | EC: 3.4.21 | | serine-type |
| | | | | | | | endopeptidase activity |
| 147459_at | 0.021 | −7.0 | FBgn0034382 | CG18609 | — | | |
| 146569_at | 0.022 | −7.1 | FBgn0032913 | CG9259 | — | | |
| 151776_at | 0.020 | −7.6 | FBgn0037763 | CG16904 | — | | |
| 151781_at | 0.027 | −8.4 | FBgn0039342 | CG5107 | — | | |
| 150206_at | 0.021 | −8.8 | FBgn0038718 | CG17752 | — | | transporter activity |
| 152623_at | 0.023 | −9.3 | FBgn0015570 | Est2 | EC: 3.1.1.1 | | carboxylesterase activity |
| 147334_at | 0.023 | −9.7 | FBgn0034160 | CG5550 | — | | |
| 149767_at | 0.021 | −9.9 | FBgn0038033 | CG10097 | — | | |
| 152313_at | 0.026 | −10.1 | FBgn0015714 | Cyp6a17 | EC: 1.14.14.1 | electron transport | cytochrome P450 activity |

TABLE 20
The Top 20 Down-Regulated and Top 20 Up-Regulated Genes in
Response to dPTEN Overexpression Ranked by Fold Change

| | SAM | Fold | | Gene | | Biological Process | |
|---|---|---|---|---|---|---|---|
| Probe Set | q-value | change | FBgn | Symbol | EC Number | (GO) | Molecular Function (GO) |
| 144474_at | 0.012 | 8.7 | FBgn0029703 | CG12692 | — | | |
| 143239_at | 0.012 | 8.0 | FBgn0002565 | Lsp2 | — | | nutrient reservoir activity |
| 153741_at | 0.012 | 6.5 | FBgn0028509 | cenG1A | — | small GTPase | ARF GTPase activator |
| | | | | | | mediated signal | activity |
| | | | | | | transduction | |
| 148259_at | 0.011 | 6.0 | FBgn0035673 | CG6602 | — | | |
| 151514_at | 0.012 | 5.3 | GH12677.3 | — | — | | |
| | | | prime-hit | | | | |
| 142040_at | 0.014 | 5.0 | LD33980.3 | CG10623 | — | | |
| | | | prime-hit | | | | |
| 142259_at | 0.013 | 4.6 | FBgn0000028 | acj6 | — | synaptic target | RNA polymerase II |
| | | | | | | recognition | transcription factor activity |
| 151899_at | 0.013 | 4.5 | FBgn0036992 | CG11796 | EC: 1.13.11.27 | | 4-hydroxyphenylpyruvate |
| | | | | | | | dioxygenase activity |
| 151644_at | 0.012 | 4.5 | LD16427.3 | — | — | | |
| | | | prime-hit | | | | |
| 154969_at | 0.014 | 4.2 | FBgn0037644 | CG11964 | — | | |
| 152021_at | 0.013 | 4.2 | FBgn0004237 | Hrb87F | — | | RNA binding activity |
| 153539_at | 0.013 | 4.2 | FBgn0026077 | Gasp | — | | structural constituent of |
| | | | | | | | peritrophic membrane |
| | | | | | | | (sensu Insecta) |
| 142441_at | 0.012 | 4.1 | FBgn0001122 | G-o&agr; | EC: 3.6.1.46 | G-protein coupled | heterotrimeric G-protein |
| | | | | 47A | | receptor protein | GTPase activity |
| | | | | | | signaling pathway | |
| 147491_at | 0.012 | 3.8 | FBgn0034435 | CG9975 | — | | |
| 147598_at | 0.014 | 3.7 | FBgn0034581 | CG3986 | — | | |
| 143858_at | 0.014 | 3.7 | FBgn0015924 | crq | — | defense response | scavenger receptor activity |
| 154590_at | 0.013 | 3.6 | FBgn0036378 | CG10046 | — | | |
| 142202_at | 0.014 | 3.5 | FBgn0034664 | CG4377 | — | | |
| 153758_at | 0.012 | 3.5 | FBgn0000338 | cnc | — | regulation of pole | RNA polymerase II |
| | | | | | | plasm oskar mRNA | transcription factor activity |
| | | | | | | localization | |
| 143588_at | 0.012 | 3.5 | FBgn0010228 | HmgZ | — | | DNA binding activity |
| 146646_at | 0.012 | 3.5 | FBgn0033028 | CG11665 | — | | monocarboxylic acid |
| | | | | | | | transporter activity |
| 152129_at | 0.014 | 3.3 | FBgn0027569 | BcDNA: | EC: 2.7.1.37 | | protein serine |
| | | | | GH07688 | | | |
| 149230_at | 0.013 | −5.5 | FBgn0037163 | CG11440 | EC: 3.1.3.4 | dephosphorylation | phosphatidate phosphatase |
| | | | | | | | activity |
| 150206_at | 0.012 | −5.5 | FBgn0038718 | CG17752 | — | | transporter activity |
| 149206_at | 0.012 | −5.7 | FBgn0037126 | CG14567 | — | | |
| 152137_at | 0.012 | −5.9 | FBgn0039754 | CG9747 | — | | acyl-CoA delta(11)- |
| | | | | | | | desaturase activity |
| 142139_at | 0.013 | −6.0 | FBgn0029549 | CG3699 | — | | |
| 150947_at | 0.014 | −6.1 | FBgn0039853 | CG11518 | — | | |
| 148056_at | 0.012 | −6.1 | FBgn0035358 | CG14949 | — | | |
| 149329_at | 0.012 | −6.4 | FBgn0037323 | CG2663 | — | | tocopherol binding activity |
| 151094_at | 0.012 | −6.4 | FBgn0040609 | CG3348 | — | | |
| 143295_at | 0.012 | −6.6 | FBgn0003046 | Pcp | — | | structural constituent of |
| | | | | | | | pupal cuticle (sensu |
| | | | | | | | Insecta) activity |
| 145474_at | 0.012 | −6.9 | FBgn0031176 | CG1678 | — | | |
| 147409_at | 0.012 | −7.1 | FBgn0034294 | CG5765 | — | | |
| 154890_at | 0.012 | −7.1 | FBgn0036289 | CG10657 | — | | retinal binding activity |
| 148553_at | 0.012 | −7.1 | FBgn0036129 | CG6261 | — | | |
| 141450_at | 0.011 | −7.2 | FBgn0024289 | Sodh-1 | EC: 1.1.1.14 | | L-iditol 2-dehydrogenase |
| | | | | | | | activity |
| 153258_at | 0.012 | −7.6 | FBgn0037999 | CG4860 | EC: 1.3.99.3 | | butyryl-CoA dehydrogenase |
| | | | | | | | activity |
| 152049_at | 0.013 | −7.8 | FBgn0028493 | BcDNA: | — | | |
| | | | | GH06048 | | | |
| 143038_at | 0.012 | −8.1 | FBgn0037207 | Mes2 | — | | |
| 143437_at | 0.013 | −8.8 | FBgn0004181 | Peb | — | | |
| 147520_at | 0.012 | −8.9 | FBgn0034474 | Obp56g | — | | odorant binding activity |
| 143789_at | 0.012 | −9.4 | FBgn0015279 | Pi3K92E | EC: 2.7.1.137 | phosphorylation | phosphatidylinositol |
| | | | | | | | 3-kinase activity |
| 147334_at | 0.012 | −32.1 | FBgn0034160 | CG5550 | — | | |

TABLE 21
The Top 20 Down-Regulated and Top 20 Up-Regulated Genes in Response
to Dp110^{D954A} Overexpression Ranked by Fold Change

| | SAM | Fold | | Gene | | Biological Process | |
|---|---|---|---|---|---|---|---|
| Probe Set | q-value | change | FBgn | Symbol | EC Number | (GO) | Molecular Function (GO) |
| 143239_at | 0.005 | 11.3 | FBgn0002565 | Lsp2 | — | | nutrient reservoir activity |
| 148259_at | 0.007 | 7.0 | FBgn0035673 | CG6602 | — | | |
| 146054_at | 0.015 | 5.4 | FBgn0032080 | CG9525 | — | | |
| 153741_at | 0.007 | 4.6 | FBgn0028509 | cenG1A | — | small GTPase | ARF GTPase activator |
| | | | | | | mediated signal | activity |
| | | | | | | transduction | |
| 152747_at | 0.006 | 4.4 | FBgn0011828 | Pxn | EC: 1.11.1.7 | | peroxidase activity |
| 151598_at | 0.018 | 4.2 | HL01868.3 | CG10077 | — | | |
| | | | prime-hit | | | | |
| 153758_at | 0.003 | 3.7 | FBgn0000338 | cnc | — | regulation of pole | RNA polymerase II |
| | | | | | | plasm oskar mRNA | transcription factor activity |
| | | | | | | localization | |
| 143162_at | 0.020 | 3.6 | FBgn0000711 | flw | EC: 3.1.3.16 | muscle attachment | protein phosphatase type 1, |
| | | | | | | | catalyst activity |
| 153539_at | 0.010 | 3.6 | FBgn0026077 | Gasp | — | | structural constituent |
| | | | | | | | of peritrophic membrane |
| | | | | | | | (sensu Insecta) |
| 153370_at | 0.005 | 3.5 | FBgn0035467 | CG1079 | — | | |
| 154944_at | 0.017 | 3.5 | FBgn0030856 | CG8267 | — | | |
| 152093_at | 0.016 | 3.4 | FBgn0031674 | CG5822 | — | | |
| 145018_at | 0.012 | 3.2 | FBgn0030498 | CG15759 | — | | |
| 153383_at | 0.012 | 3.1 | FBgn0037277 | CG17735 | — | | ligand-dependent nuclear |
| | | | | | | | receptor interactor activity |
| 151899_at | 0.014 | 3.0 | FBgn0036992 | CG11796 | EC: 1.13.11.27 | | 4-hydroxyphenylpyruvate |
| | | | | | | | dioxygenase activity |
| 151499_at | 0.011 | 3.0 | GH08192.3 | — | — | | |
| | | | prime-hit | | | | |
| 153833_at | 0.016 | 2.9 | FBgn0029873 | CG3918 | — | | nucleic acid binding activity |
| 152189_at | 0.013 | 2.9 | FBgn0038475 | Keap1 | — | | actin binding activity |
| 154042_at | 0.003 | 2.9 | FBgn0027835 | Dp1 | — | | single-stranded DNA |
| | | | | | | | binding activity |
| 152303_at | 0.005 | 2.8 | FBgn0030183 | CG15309 | — | | |
| 143145_at | 0.007 | 2.8 | FBgn0000562 | egl | — | oocyte cell fate | nucleic acid binding activity |
| | | | | | | determination | |
| 142793_at | 0.005 | 2.8 | FBgn0004624 | CaMKII | EC: 2.7.1.123 | neuromuscular | protein serine |
| | | | | | | junction | |
| | | | | | | development | |
| 153724_at | 0.006 | −5.1 | FBgn0039244 | CG11069 | EC: 3.6.3 | | ATP-binding cassette (ABC) |
| | | | | | | | transporter activity |
| 146331_at | 0.003 | −5.2 | FBgn0032505 | CG16826 | — | | |
| 148640_at | 0.004 | −5.2 | FBgn0036264 | CG11529 | EC: 3.4.21 | | trypsin activity |
| 144320_at | 0.006 | −5.3 | FBgn0028936 | BG: | — | | |
| | | | | DS00180.9 | | | |
| 150717_at | 0.003 | −5.5 | FBgn0039495 | CG5909 | EC: 3.4.21 | | serine-type endopeptidase |
| | | | | | | | activity |
| 153258_at | 0.005 | −5.5 | FBgn0037999 | CG4860 | EC: 1.3.99.3 | | butyryl-CoA dehydrogenase |
| | | | | | | | activity |
| 148553_at | 0.004 | −5.7 | FBgn0036129 | CG6261 | — | | |
| 147520_at | 0.006 | −6.1 | FBgn0034474 | Obp56g | — | | odorant binding activity |
| 147409_at | 0.005 | −6.2 | FBgn0034294 | CG5765 | — | | |
| 148056_at | 0.005 | −6.2 | FBgn0035358 | CG14949 | — | | |
| 144239_at | 0.003 | −6.2 | FBgn0028583 | Ics | — | | |
| 147903_at | 0.005 | −6.3 | FBgn0035089 | CG9358 | — | | |
| 143295_at | 0.005 | −6.6 | FBgn0003046 | Pcp | — | | structural constituent of |
| | | | | | | | pupal cuticle (sensu |
| | | | | | | | Insecta) activity |
| 145474_at | 0.003 | −6.8 | FBgn0031176 | CG1678 | — | | |
| 151094_at | 0.005 | −7.3 | FBgn0040609 | CG3348 | — | | |
| 149206_at | 0.006 | −7.4 | FBgn0037126 | CG14567 | — | | |
| 154890_at | 0.005 | −8.4 | FBgn0036289 | CG10657 | — | | retinal binding activity |
| 149791_at | 0.005 | −9.1 | FBgn0038070 | CG6753 | EC: 3.1.1.3 | | triacylglycerol lipase activity |
| 150947_at | 0.006 | −12.7 | FBgn0039853 | CG11518 | — | | |
| 147334_at | 0.005 | −14.1 | FBgn0034160 | CG5550 | — | | |

TABLE 22
The Top 20 Down-Regulated and Top 20 Up-Regulated Genes in Response to dfoxo Overexpression Ranked by Fold Change

| | SAM | Fold | | Gene | | Biological Process | |
|---|---|---|---|---|---|---|---|
| Probe Set | q-value | Change | FBgn | Symbol | EC Number | (GO) | Molecular Function (GO) |
| 146850_at | 0.005 | 27.0 | FBgn0033355 | CG13748 | — | | serine protease |
| | | | | | | | inhibitor activity |
| 143942_at | 0.006 | 24.2 | FBgn0020765 | Acp65Aa | — | | structural constituent of adult |
| | | | | | | | cuticle (sensu Insecta) |
| | | | | | | | activity |
| 154014_at | 0.005 | 23.8 | FBgn0032130 | CG3838 | — | | DNA binding activity |
| 146180_at | 0.005 | 19.5 | FBgn0032281 | CG17107 | — | | |
| 150552_at | 0.005 | 19.0 | FBgn0039273 | CG9996 | — | | transferase activity, |
| | | | | | | | transferring hexosyl groups |
| 148259_at | 0.005 | 14.5 | FBgn0035673 | CG6602 | — | | |
| 149394_at | 0.006 | 13.3 | FBgn0037429 | CG15189 | — | | |
| 153029_at | 0.006 | 11.2 | FBgn0005633 | fln | — | | |
| 143747_at | 0.006 | 10.3 | FBgn0014033 | Sr-Cl | — | response to bacteria | scavenger receptor activity |
| 153761_at | 0.005 | 10.1 | FBgn0039241 | CG11089 | EC: 2.1.2.3 | | phosphoribosylamino- |
| | | | | | | | imidazole-carboxamide |
| | | | | | | | formyltransferase activity |
| 141426_at | 0.006 | 8.5 | FBgn0036996 | CG5932 | EC: 3.1.1.3 | | triacylglycerol lipase activity |
| 146645_at | 0.005 | 8.4 | FBgn0033027 | CG12408 | — | | calcium ion binding activity |
| 151899_at | 0.005 | 7.3 | FBgn0036992 | CG11796 | EC: 1.13.11.27 | | 4-hydroxyphenylpyruvate |
| | | | | | | | dioxygenase activity |
| 142318_at | 0.006 | 7.1 | FBgn0037433 | CG17919 | — | | phosphatidylethanolamine |
| | | | | | | | binding activity |
| 141213_at | 0.006 | 6.5 | FBgn0033728 | CG8505 | — | | structural constituent of |
| | | | | | | | cuticle (sensu Insecta) |
| | | | | | | | activity |
| 141353_at | 0.005 | 6.5 | FBgn0038299 | CG6687 | — | | serpin |
| 146256_at | 0.005 | 6.2 | FBgn0032381 | CG14934 | EC: 3.2.1.20 | | alpha-amylase activity |
| 143605_at | 0.005 | 5.8 | FBgn0010381 | Drs | — | antifungal humoral | antifungal peptide activity |
| | | | | | | response (sensu | |
| | | | | | | Invertebrata) | |
| 142969_at | 0.005 | 5.7 | FBgn0004629 | Cys | — | | cysteine protease |
| | | | | | | | inhibitor activity |
| 154884_at | 0.005 | 5.5 | FBgn0035763 | CG8602 | — | | transporter activity |
| 153741_at | 0.006 | 5.5 | FBgn0028509 | cenG1A | — | small GTPase | ARF GTPase activator |
| | | | | | | mediated signal | activity |
| | | | | | | transduction | |
| 148253_at | 0.005 | −11.1 | FBgn0035664 | CG6467 | EC: 3.4.21 | | serine-type endopeptidase |
| | | | | | | | activity |
| 147118_at | 0.005 | −11.6 | FBgn0033774 | CG12374 | EC: 3.4.17.1 | | carboxypeptidase A activity |
| 152644_at | 0.006 | −12.4 | FBgn0038398 | CG4979 | EC: 3.1.1.32 | | phosphatidylserine-specific |
| | | | | | | | phospholipase A1 activity |
| 143437_at | 0.006 | −12.4 | FBgn0004181 | Peb | — | | |
| 142538_at | 0.005 | −13.2 | FBgn0034341 | CG17531 | EC: 2.5.1.18 | | glutathione transferase |
| | | | | | | | activity |
| 147334_at | 0.006 | −14.6 | FBgn0034160 | CG5550 | — | | |
| 148048_at | 0.006 | −15.1 | FBgn0035343 | CG16762 | — | | |
| 151776_at | 0.005 | −15.3 | FBgn0037763 | CG16904 | — | | |
| 152448_at | 0.005 | −20.1 | FBgn0031860 | CG11236 | EC: 1.4.3.1 | | oxidoreductase activity |
| 149230_at | 0.006 | −22.7 | FBgn0037163 | CG11440 | EC: 3.1.3.4 | dephosphorylation | phosphatidate phosphatase |
| | | | | | | | activity |
| 149767_at | 0.005 | −22.9 | FBgn0038033 | CG10097 | — | | |
| 146902_at | 0.005 | −24.1 | FBgn0033444 | CG1652 | — | | |
| 143984_at | 0.005 | −24.2 | FBgn0023415 | Acp32CD | — | negative regulation | hormone activity |
| | | | | | | of female receptivity, | |
| | | | | | | post-mating | |
| 152849_at | 0.006 | −25.4 | FBgn0033445 | CG1656 | — | | |
| 144006_at | 0.005 | −28.5 | FBgn0023550 | EG: | — | | |
| | | | | 103B4.2 | | | |
| 150699_at | 0.005 | −28.5 | FBgn0039471 | CG6295 | EC: 3.1.1.3 | | enzyme activity |
| 150575_at | 0.006 | −29.4 | FBgn0039310 | CG11878 | — | | |
| 154985_at | 0.005 | −37.1 | FBgn0035077 | CG9083 | — | | |
| 147520_at | 0.006 | −39.3 | FBgn0034474 | Obp56g | — | | odorant binding activity |
| 150574_at | 0.005 | −43.5 | FBgn0039309 | CG11891 | — | | |
| 142419_at | 0.005 | −48.0 | FBgn0030098 | CG12057 | — | | |

TABLE 23
The top 20 Down-regulated and top 20 up-regulated genes in response to dPDK1 over-expression ranked by fold change

| | SAM | Fold | | Gene | | Biological Process | |
|---|---|---|---|---|---|---|---|
| Probe Set | q-value | Change | FBgn | Symbol | EC Number | (GO) | Molecular Function (GO) |
| 143781_at | 0.020 | 20.3 | FBgn0015035 | Cyp4e3 | — | electron transport | cytochrome P450 activity |
| 149938_at | 0.015 | 7.9 | FBgn0038291 | CG3984 | — | | |
| 150261_at | 0.020 | 7.5 | FBgn0038795 | CG4335 | EC: 1.14.11.1 | | gamma-butyrobetaine, |
| | | | | | | | 2-oxoglutarate |
| | | | | | | | dioxygenase activity |
| 141374_at | 0.015 | 6.8 | FBgn0012042 | AttA | — | antibacterial humoral | antibacterial peptide activity |
| | | | | | | response (sensu | |
| | | | | | | Invertebrata) | |
| 152313_at | 0.015 | 6.4 | FBgn0015714 | Cyp6a17 | EC: 1.14.14.1 | electron transport | cytochrome P450 activity |
| 142677_s_at | 0.018 | 5.3 | FBgn0020386 | Pk61C | EC: 2.7.1.37 | protein amino acid | protein serine |
| | | | | | | phosphorylation | |
| 146461_at | 0.017 | 4.8 | FBgn0032726 | CG10621 | EC: 2.1.1 | | homocysteine |
| | | | | | | | S-methyltransferase activity |
| 143470_at | 0.015 | 4.4 | FBgn000443l | LysX | EC: 3.2.1.17 | antimicrobial humoral | lysozyme activity |
| | | | | | | response (sensu | |
| | | | | | | Invertebrata) | |
| 143807_at | 0.015 | 4.3 | FBgn0015402 | ksr | EC: 2.7.1.37 | protein amino acid | protein serine |
| | | | | | | phosphorylation | |
| 150588_at | 0.017 | 4.3 | FBgn0039330 | CG11909 | EC: 3.2.1.20 | | hydrolase activity, |
| | | | | | | | hydrolyzing O-glycosyl |
| | | | | | | | compounds |
| 143770_at | 0.015 | 3.9 | FBgn0014865 | Mtk | — | antifungal humoral | Gram-positive antibacterial |
| | | | | | | response (sensu | peptide activity |
| | | | | | | Invertebrata) | |
| 148310_i_at | 0.015 | 3.8 | FBgn0035744 | CG8628 | — | cell acyl-CoA | acyl-CoA binding activity |
| | | | | | | homeostasis | |
| 148352_at | 0.019 | 3.7 | FBgn0035806 | PGRP-SD | — | immune response | N-acetylmuramoyl-L-alanine |
| | | | | | | | amidase activity |
| 150398_at | 0.016 | 3.7 | FBgn0039022 | CG4725 | — | | endothelin-converting |
| | | | | | | | enzyme activity |
| 145647_at | 0.015 | 3.5 | FBgn0031432 | Cyp309a1 | — | electron transport | cytochrome P450 activity |
| 143699_at | 0.018 | 3.4 | FBgn0011834 | Ser6 | EC: 3.4.16 | | serine-type endopeptidase |
| | | | | | | | activity |
| 150050_at | 0.022 | 3.4 | FBgn0038481 | CG17475 | EC: 3.4.21 | | trypsin activity |
| 149808_at | 0.015 | 3.3 | FBgn0038095 | Cyp304a1 | — | electron transport | cytochrome P450 activity |
| 144220_at | 0.015 | 3.2 | FBgn0028510 | BG: | — | negative regulation | protein biosynthesis |
| | | | | DS07851.3 | | of protein | inhibitor activity |
| | | | | | | biosynthesis | |
| 145253_at | 0.017 | 3.2 | FBgn0030859 | CG12990 | — | | transferase activity, |
| | | | | | | | transferring groups other |
| | | | | | | | than amino-acyl groups |
| 148573_at | 0.018 | 3.2 | FBgn0036157 | CG7560 | EC: 1.5.1.20 | | methylenetetrahydrofolate |
| | | | | | | | reductase (NADPH) activity |
| 145847_at | 0.016 | −2.4 | FBgn0031741 | CG11034 | EC: 3.4.14.5 | | dipeptidyl-peptidase |
| | | | | | | | IV activity |
| 143476_at | 0.030 | −2.4 | FBgn0004513 | Mdr65 | EC: 3.6.3.44 | | multidrug transporter activity |
| 142217_at | 0.017 | −2.4 | FBgn0011276 | HLH3B | — | | transcription factor activity |
| 153476_at | 0.021 | −2.5 | FBgn0036907 | CG8533 | — | | glutamate-gated ion |
| | | | | | | | channel activity |
| 143272_at | 0.028 | −2.6 | FBgn0002855 | Acp26Aa | — | oviposition | hormone activity |
| 153085_at | 0.023 | −2.6 | FBgn0014019 | Rh5 | — | G-protein coupled | short-wave-sensitive opsin |
| | | | | | | receptor protein | |
| | | | | | | signaling pathway | |
| 142279_at | 0.016 | −2.8 | FBgn0000241 | bw | EC: 3.6.3 | pteridine | ATP-binding cassette (ABC) |
| | | | | | | biosynthesis | transporter activity |
| 143485_at | 0.023 | −2.9 | FBgn0004575 | Syn | — | neurotransmitter | |
| | | | | | | secretion | |
| 141511_at | 0.025 | −2.9 | FBgn0016794 | dos | — | sevenless receptor | SH3 |
| | | | | | | signaling pathway | |
| 145319_at | 0.027 | −3.0 | FBgn0030948 | Cyp306a1 | — | electron transport | cytochrome P450 activity |
| 143787_at | 0.021 | −3.0 | FBgn0015271 | Orc5 | — | mitotic chromosome | DNA binding activity |
| | | | | | | condensation | |
| 143320_at | 0.025 | −3.1 | FBgn0003248 | Rh2 | — | G-protein coupled | opsin |
| | | | | | | receptor protein | |
| | | | | | | signaling pathway | |
| 154188_at | 0.015 | −3.5 | FBgn0030817 | CG4991 | — | | amino acid-polyamine |
| | | | | | | | transporter activity |
| 148150_at | 0.021 | −3.5 | FBgn0035505 | CG15004 | — | | sodium channel auxiliary |
| | | | | | | | protein activity |
| 149775_at | 0.018 | −3.7 | FBgn0038045 | Neu5Ac | — | carbohydrate | N-acetylneuraminic acid |
| | | | | | | biosynthesis | phosphate synthase activity |
| 154692_at | 0.019 | −3.8 | FBgn0033577 | CG18239 | — | | |
| 146780_at | 0.016 | −3.9 | FBgn0033242 | CG2916 | — | | |
| 145820_at | 0.015 | −4.5 | FBgn0031701 | TotM | — | humoral defense | |
| | | | | | | mechanism (sensu | |
| | | | | | | Invertebrata) | |
| 154548_at | 0.022 | −4.6 | FBgn0034098 | CG15707 | — | | nucleic acid binding activity |
| 154821_at | 0.019 | −5.3 | FBgn0037699 | CG8147 | — | | |
| 148087_at | 0.015 | −5.7 | FBgn0035412 | CG14957 | — | | chitin binding activity |
| 151740_at | 0.015 | −6.1 | SD02216.3 | — | — | | |
| | | | prime-hit | | | | |
| 151630_at | 0.023 | −10.3 | LD10776.3 | — | — | | |
| | | | prime-hit | | | | |

TABLE 24
The Top 20 Down-Regulated and Top 20 Up-Regulated Genes in Response to dS6K Overexpression Ranked by Fold Change

| | SAM | Fold | | Gene | | Biological Process | |
|---|---|---|---|---|---|---|---|
| Probe Set | q-value | Change | FBgn | Symbol | EC Number | (GO) | Molecular Function (GO) |
| 143942_at | 0.005 | 46.7 | FBgn0020765 | Acp65Aa | — | | structural constituent |
| | | | | | | | of adult cuticle (sensu |
| | | | | | | | Insecta) activity |
| 148762_at | 0.006 | 27.2 | FBgn0036440 | CG17177 | — | | |
| 149624_at | 0.014 | 18.7 | FBgn0037801 | CG3999 | EC: 1.4.4.2 | | glycine dehydrogenase |
| | | | | | | | (decarboxylating) activity |
| 150273_at | 0.005 | 14.1 | FBgn0038819 | CG5494 | — | | structural constituent |
| | | | | | | | of cuticle (sensu |
| | | | | | | | Insecta) activity |
| 150206_at | 0.005 | 13.8 | FBgn0038718 | CG17752 | — | | transporter activity |
| 145450_at | 0.005 | 12.6 | FBgn0031141 | CG1304 | EC: 3.4.21 | | trypsin activity |
| 152313_at | 0.005 | 9.2 | FBgn0015714 | Cyp6a17 | EC: 1.14.14.1 | electron transport | cytochrome P450 activity |
| 143944_at | 0.006 | 8.8 | FBgn0020906 | Ser4 | EC: 3.4.21 | | serine-type endopeptidase |
| | | | | | | | activity |
| 145236_at | 0.006 | 8.6 | FBgn0030829 | CG12998 | — | | |
| 148870_at | 0.005 | 8.2 | FBgn0036597 | CG4962 | — | | |
| 147237_at | 0.006 | 8.0 | FBgn0034003 | CG8094 | — | | |
| 149799_at | 0.011 | 7.0 | FBgn0038083 | CG5999 | EC: 2.4.1.17 | | glucuronosyltransferase |
| | | | | | | | activity |
| 150205_at | 0.006 | 6.8 | FBgn0038717 | CG17751 | — | | transporter activity |
| 141213_at | 0.006 | 5.9 | FBgn0033728 | CG8505 | — | | structural constituent |
| | | | | | | | of cuticle (sensu |
| | | | | | | | Insecta) activity |
| 145795_at | 0.006 | 5.9 | FBgn0031653 | CG8871 | EC: 3.4.21.1 | | trypsin activity |
| 143759_at | 0.006 | 5.6 | FBgn0014454 | Acp1 | — | | structural constituent |
| | | | | | | | of adult cuticle (sensu |
| | | | | | | | Insecta) activity |
| 150261_at | 0.015 | 5.1 | FBgn0038795 | CG4335 | EC: 1.14.11.1 | | gamma-butyrobetaine,2- |
| | | | | | | | oxoglutarate dioxygenase |
| | | | | | | | activity |
| 151932_at | 0.006 | 4.9 | FBgn0000406 | Cyt-b5-r | — | electron transport | electron transporter activity |
| 143781_at | 0.007 | 4.8 | FBgn0015035 | Cyp4e3 | — | electron transport | cytochrome P450 activity |
| 143876_at | 0.006 | 4.3 | FBgn0016675 | Lectin- | — | defense response | galactose binding activity |
| | | | | galC1 | | | |
| 141450_at | 0.005 | 4.3 | FBgn0024289 | Sodh-1 | EC: 1.1.1.14 | | L-iditol 2-dehydrogenase |
| | | | | | | | activity |
| 152766_at | 0.005 | −3.0 | FBgn0002563 | Lsp1&bgr; | — | | nutrient reservoir activity |
| 144332_at | 0.014 | −3.3 | FBgn0028950 | BG: | EC: 3.4.24 | | metalloendopeptidase |
| | | | | BACR44L22.1 | | | activity |
| 143603_i_at | 0.005 | −3.3 | FBgn0010359 | &ggr; Try | EC: 3.4.21.4 | proteolysis and | trypsin activity |
| | | | | | | peptidolysis | |
| 141435_at | 0.006 | −3.5 | FBgn0031249 | CG11911 | EC: 3.4.21 | | trypsin activity |
| 148401_at | 0.006 | −3.5 | FBgn0035887 | CG7170 | EC: 3.4.21.1 | | chymotrypsin activity |
| 152026_at | 0.016 | −3.6 | FBgn0027584 | BcDNA: | EC: 3.1.1.1 | | carboxylesterase activity |
| | | | | GH05741 | | | |
| 148150_at | 0.010 | −3.7 | FBgn0035505 | CG15004 | — | | sodium channel auxiliary |
| | | | | | | | protein activity |
| 141389_at | 0.014 | −3.7 | FBgn0034582 | CG10531 | EC: 3.2.1.14 | | hydrolase activity |
| 154188_at | 0.006 | −3.9 | FBgn0030817 | CG4991 | — | | amino acid-polyamine |
| | | | | | | | transporter activity |
| 143470_at | 0.006 | −4.6 | FBgn0004431 | LysX | EC: 3.2.1.17 | antimicrobial | lysozyme activity |
| | | | | | | humoral response | |
| | | | | | | (sensu Invertebrata) | |
| 143112_at | 0.006 | −4.7 | FBgn0000360 | Cp38 | — | insect chorion | structural constituent of |
| | | | | | | formation | chorion (sensu Insecta) |
| 142217_at | 0.005 | −4.7 | FBgn0011276 | HLH3B | — | | transcription factor activity |
| 142220_at | 0.006 | −5.0 | FBgn0033367 | CG8193 | EC: 1.14.18.1 | defense response | monophenol |
| | | | | | | | monooxygenase activity |
| 155132_at | 0.026 | −5.3 | FBgn0000520 | dwg | — | | transcription factor activity |
| 141245_at | 0.012 | −6.3 | FBgn0014861 | Mcm2 | — | cell proliferation | chromatin binding activity |
| 149491_at | 0.009 | −7.0 | FBgn0037577 | CG7443 | — | | |
| 148309_at | 0.006 | −7.1 | FBgn0035743 | CG15829 | — | cell acyl-CoA | acyl-CoA binding activity |
| | | | | | | homeostasis | |
| 144590_at | 0.016 | −8.2 | FBgn0029860 | CG15891 | — | | |
| 146093_at | 0.009 | −8.8 | FBgn0032127 | CG13114 | — | | |
| 142368_at | 0.012 | −10.3 | FBgn0037196 | CG8220 | — | | |
| 148400_at | 0.005 | −10.3 | FBgn0035886 | CG7118 | EC: 3.4.21 | | trypsin activity |

TABLE 25
Human Homologs of Drosophila Genes Regulated by ISP

| Human gene | FlyBase | | | | Affy chip |
|---|---|---|---|---|---|
| Locus ID | GeneID | FlyGene CG | Blast Prob | Pct Ident | Probe Set |
| 6767 | FBgn0029676 | CG2947 | 6E−72 | 40 | 141203_at |
| 79903 | FBgn0036039 | CG18177 | 5E−57 | 50 | 141205_at |
| 5447 | FBgn0015623 | CG11567 | 0 | 59 | 141208_at |
| 537 | FBgn0004868 | CG4422 | 1E−177 | 67 | 141214_at |
| 26528 | FBgn0004838 | CG10377 | 4E−62 | 36 | 141218_at |
| 2975 | FBgn0032517 | CG7099 | 3E−40 | 21 | 141219_at |
| 51154 | FBgn0033485 | CG1381 | 4E−70 | 54 | 141230_at |
| 5408 | FBgn0029831 | CG5966 | 9E−63 | 35 | 141233_at |
| 4171 | FBgn0014861 | CG7538 | 0 | 64 | 141245_at |
| 7508 | FBgn0004698 | CG8153 | 1E−100 | 38 | 141256_at |
| 54504 | FBgn0038738 | CG4572 | 1E−103 | 44 | 141260_at |
| 994 | FBgn0003525 | CG1395 | 3E−52 | 37 | 141264_at |
| 9380 | FBgn0037370 | CG1236 | 2E−90 | 53 | 141292_at |
| 55186 | FBgn0031359 | CG18317 | 8E−85 | 49 | 141322_at |
| 1738 | FBgn0036762 | CG7430 | 0 | 67 | 141356_at |
| 6204 | FBgn0031035 | CG14206 | 7E−52 | 63 | 141358_at |
| 1209 | FBgn0031590 | CG3702 | 0 | 56 | 141359_at |
| 3938 | FBgn0036659 | CG9701 | 1E−125 | 47 | 141360_at |
| 5298 | FBgn0004373 | CG7004 | 1E−171 | 42 | 141383_at |
| 63826 | FBgn0037684 | CG8129 | 3E−43 | 36 | 141406_at |
| 23406 | FBgn0030955 | CG6891 | 6E−29 | 43 | 141412_at |
| 1058 | FBgn0037971 | CG10007 | 2E−50 | 35 | 141417_at |
| 10134 | FBgn0035165 | CG13887 | 1E−41 | 39 | 141434_at |
| 51380 | FBgn0000153 | CG7811 | 1E−151 | 52 | 141440_at |
| 6652 | FBgn0024289 | CG1982 | 4E−84 | 54 | 141450_at |
| 4547 | FBgn0032904 | CG9342 | 7E−60 | 24 | 141458_at |
| 212 | FBgn0020764 | CG3017 | 1E−155 | 56 | 141461_at |
| 9846 | FBgn0016794 | CG1044 | 1E−24 | 39 | 141511_at |
| 64699 | FBgn0023479 | CG4821 | 1E−44 | 36 | 141527_at |
| 60487 | FBgn0037250 | CG1074 | 5E−97 | 41 | 141546_at |
| 2539 | FBgn0004057 | CG12529 | 0 | 65 | 141547_at |
| 90850 | FBgn0035024 | CG11414 | 1E−103 | 29 | 141554_at |
| 25912 | FBgn0034172 | CG6665 | 4E−27 | 39 | 141562_at |
| 54676 | FBgn0037391 | CG2017 | 1E−148 | 51 | 141564_at |
| 2764 | FBgn0028894 | CG5869 | 6E−40 | 53 | 141567_at |
| 9909 | FBgn0025864 | CG12737 | 1E−130 | 37 | 141584_at |
| 2354 | FBgn0001297 | CG15509 | 7E−18 | 42 | 141592_at |
| 347734 | FBgn0038524 | CG7623 | 1E−101 | 48 | 141596_at |
| 80303 | FBgn0032731 | CG10641 | 3E−56 | 59 | 141598_at |
| 5829 | FBgn0051794 | CG31794 | 1E−140 | 76 | 141605_at |
| 55697 | FBgn0038058 | CG5608 | 1E−112 | 41 | 141609_at |
| 60412 | FBgn0037373 | CG2095 | 1E−155 | 34 | 141611_at |
| 3843 | FBgn0011341 | CG1059 | 0 | 52 | 141618_at |
| 10999 | FBgn0021953 | CG7400 | 1E−162 | 47 | 141619_at |
| 23301 | FBgn0034180 | CG15609 | 1E−63 | 27 | 141621_at |
| 1173 | FBgn0024832 | CG7057 | 0 | 87 | 141628_at |
| 206358 | FBgn0032036 | CG13384 | 5E−79 | 43 | 141633_at |
| 1653 | FBgn0015075 | CG9054 | 0 | 59 | 141634_at |
| 84706 | FBgn0030478 | CG1640 | 1E−156 | 55 | 141639_at |
| 55622 | FBgn0036772 | CG5290 | 4E−78 | 33 | 141652_at |
| 3329 | FBgn0015245 | CG12101 | 0 | 73 | 141664_at |
| 6935 | FBgn0004606 | CG1322 | 7E−75 | 28 | 141676_at |
| 10642 | FBgn0030235 | CG1691 | 2E−89 | 41 | 141681_at |
| 7469 | FBgn0038872 | CG5874 | 2E−66 | 37 | 141683_at |
| 10390 | FBgn0033844 | CG6016 | 1E−120 | 54 | 141694_at |
| 81502 | FBgn0031260 | CG11840 | 1E−130 | 62 | 141698_at |
| 112724 | FBgn0033205 | CG2064 | 2E−86 | 54 | 141701_at |
| 23524 | FBgn0035253 | CG7971 | 6E−85 | 28 | 141708_at |
| 7278 | FBgn0003885 | CG2512 | 0 | 97 | 141710_r_at |
| 4285 | FBgn0033038 | CG7791 | 0 | 49 | 141715_at |
| 55278 | FBgn0027658 | CG6007 | 1E−147 | 52 | 141724_at |
| 8399 | FBgn0039655 | CG14507 | 0.000000000000001 | 35 | 141728_at |
| 50999 | FBgn0030606 | CG9053 | 5E−42 | 41 | 141747_at |
| 80206 | FBgn0052030 | CG32030 | 1E−173 | 50 | 141748_at |
| 5426 | FBgn0020756 | CG6768 | 0 | 55 | 141750_at |
| 116541 | FBgn0034579 | CG9353 | 2E−18 | 45 | 141755_at |
| 9128 | FBgn0036733 | CG6322 | 1E−157 | 50 | 141764_at |
| 57143 | FBgn0035039 | CG3608 | 1E−129 | 47 | 141773_at |
| 54677 | FBgn0039543 | CG12428 | 2E−96 | 31 | 141776_at |
| 10000 | FBgn0010379 | CG4006 | 0 | 63 | 141791_at |
| 10129 | CG32045 | CG32045 | 0 | 46 | 141794_at |
| 112 | FBgn0052158 | CG32158 | 0 | 50 | 141806_at |
| 9520 | FBgn0035226 | CG1009 | 0 | 59 | 141811_at |
| 51390 | FBgn0031245 | CG3625 | 8E−44 | 38 | 141831_at |
| 292 | FBgn0003360 | CG16944 | 1E−139 | 81 | 141933_at |
| 8667 | FBgn0022023 | CG9124 | 3E−77 | 46 | 141937_at |
| 5170 | FBgn0020386 | CG1210 | 3E−43 | 42 | 141986_at |
| 375 | FBgn0010348 | CG8385 | 3E−99 | 96 | 142008_at |
| 4199 | FBgn0002719 | CG10120 | 0 | 58 | 142016_at |
| 22848 | FBgn0015772 | CG10637 | 1E−117 | 52 | 142040_at |
| 113278 | FBgn0039882 | CG11576 | 2E−76 | 36 | 142134_at |
| 4125 | FBgn0032068 | CG9466 | 0 | 40 | 142147_at |
| 51390 | FBgn0031245 | CG3625 | 8E−44 | 38 | 142165_at |
| 57577 | FBgn0031496 | CG17258 | 1E−29 | 26 | 142166_at |
| 677 | FBgn0011837 | CG4070 | 8E−38 | 46 | 142170_at |
| 81796 | FBgn0032123 | CG3811 | 1E−137 | 37 | 142176_at |
| 6886 | FBgn0011276 | CG2655 | 1E−21 | 73 | 142217_at |
| 3631 | FBgn0030553 | CG1846 | 7E−62 | 38 | 142224_at |
| 91050 | FBgn0038330 | CG14868 | 0.0000000000002 | 39 | 142229_at |
| 5592 | FBgn0000721 | CG10033 | 0 | 65 | 142251_at |
| 5458 | FBgn0000028 | CG9151 | 3E−81 | 53 | 142259_at |
| 7417 | FBgn0004363 | CG6647 | 1E−101 | 62 | 142269_at |
| 63971 | FBgn0019968 | CG8183 | 0 | 57 | 142277_at |
| 26047 | FBgn0013997 | CG6827 | 1E−180 | 32 | 142283_at |
| 117247 | FBgn0001296 | CG12286 | 6E−97 | 44 | 142293_at |
| 8774 | FBgn0028552 | CG3988 | 5E−55 | 38 | 142296_at |
| 3745 | FBgn0003383 | CG1066 | 0 | 70 | 142312_at |
| 3612 | FBgn0037063 | CG9391 | 1E−66 | 46 | 142335_at |
| 2184 | FBgn0016013 | CG14993 | 1E−144 | 60 | 142340_at |
| 8394 | FBgn0034789 | CG3682 | 1E−151 | 61 | 142409_at |
| 124936 | FBgn0030099 | CG12056 | 2E−38 | 38 | 142420_at |
| 2775 | FBgn0001122 | CG2204 | 1E−174 | 83 | 142441_at |
| 2286 | FBgn0037930 | CG14715 | 6E−37 | 57 | 142486_at |
| 292 | FBgn0003360 | CG16944 | 1E−139 | 81 | 142494_at |
| 517 | FBgn0039830 | CG1746 | 1E−41 | 64 | 142498_at |
| 8667 | FBgn0022023 | CG9124 | 3E−77 | 46 | 142505_at |
| 1655 | FBgn0035720 | CG10077 | 1E−177 | 56 | 142516_at |
| 146880 | FBgn0036511 | CG6498 | 0 | 40 | 142528_at |
| 9532 | FBgn0036505 | CG7945 | 3E−23 | 36 | 142529_s_at |
| 6117 | FBgn0010173 | CG9633 | 1E−139 | 42 | 142545_at |
| 10269 | FBgn0034176 | CG9000 | 1E−118 | 49 | 142548_at |
| 22919 | FBgn0027066 | CG3265 | 5E−82 | 78 | 142558_s_at |
| 4830 | FBgn0000150 | CG2210 | 3E−65 | 77 | 142560_at |
| 5520 | FBgn0004889 | CG6235 | 0 | 79 | 142582_at |
| 6780 | FBgn0003520 | CG5753 | 2E−63 | 31 | 142592_at |
| 55011 | FBgn0032455 | CG5792 | 4E−26 | 28 | 142601_at |
| 8799 | FBgn0034058 | CG8315 | 4E−33 | 32 | 142653_at |
| 10437 | FBgn0039099 | CG10157 | 0.000000000002 | 41 | 142672_at |
| 254122 | FBgn0032005 | CG8282 | 1E−120 | 55 | 142674_at |
| 5170 | FBgn0020386 | CG1210 | 3E−43 | 42 | 142677_s_at |
| 5255 | FBgn0030087 | CG7766 | 0 | 48 | 142687_at |
| 64754 | FBgn0011566 | CG13761 | 1E−47 | 27 | 142710_at |
| 55751 | FBgn0032172 | CG5850 | 2E−93 | 46 | 142714_at |
| 53371 | FBgn0033737 | CG8831 | 2E−95 | 39 | 142722_at |
| 3157 | FBgn0010611 | CG4311 | 1E−169 | 64 | 142736_s_at |
| 4199 | FBgn0002719 | CG10120 | 0 | 58 | 142746_at |
| 91689 | FBgn0034303 | CG17680 | 6E−16 | 58 | 142776_at |
| 57128 | FBgn0013432 | CG3717 | 1E−18 | 50 | 142778_at |
| 23327 | FBgn0036736 | CG7555 | 0 | 67 | 142789_at |
| 817 | FBgn0004624 | CG18069 | 0 | 77 | 142793_at |
| 2872 | FBgn0017581 | CG17342 | 1E−117 | 54 | 142794_at |
| 22845 | FBgn0034141 | CG8311 | 5E−47 | 31 | 142795_at |
| 7088 | FBgn0001139 | CG8384 | 0 | 61 | 142820_at |
| 8882 | FBgn0030096 | CG9060 | 1E−119 | 48 | 142846_at |
| 3704 | FBgn0031663 | CG8891 | 1E−69 | 66 | 142851_at |
| 51397 | FBgn0030323 | CG2371 | 0.0000000000002 | 28 | 142870_s_at |
| 10102 | FBgn0032646 | CG6412 | 2E−45 | 39 | 142875_at |
| 2678 | FBgn0030932 | CG6461 | 1E−109 | 45 | 142878_at |
| 3654 | FBgn0010441 | CG5974 | 2E−39 | 32 | 142881_at |
| 129138 | FBgn0036052 | CG10809 | 3E−25 | 38 | 142905_at |
| 6804 | CG31136 | CG31136 | 1E−112 | 70 | 142907_at |
| 8801 | FBgn0029118 | CG10622 | 1E−128 | 59 | 142911_at |
| 7465 | FBgn0011737 | CG4488 | 1E−104 | 43 | 142924_at |
| 2065 | FBgn0003731 | CG10079 | 1E−179 | 34 | 142966_at |
| 8470 | FBgn0033504 | CG18408 | 2E−67 | 46 | 142967_at |
| 4640 | FBgn0011673 | CG7438 | 0 | 45 | 142975_at |
| 10576 | FBgn0030086 | CG7033 | 0 | 71 | 142977_at |
| 90993 | FBgn0004396 | CG7450 | 5E−37 | 50 | 143003_at |
| 10058 | FBgn0038376 | CG4225 | 0 | 50 | 143022_at |
| 10845 | FBgn0038745 | CG4538 | 1E−177 | 58 | 143035_at |
| 60 | FBgn0000043 | CG12051 | 0 | 97 | 143059_at |
| 60 | FBgn0000047 | CG5178 | 0 | 95 | 143061_at |
| 55252 | FBgn0000142 | CG8787 | 1E−36 | 29 | 143080_at |
| 1746 | FBgn0000157 | CG3629 | 1E−37 | 40 | 143081_at |
| 56288 | FBgn0000163 | CG5055 | 3E−83 | 31 | 143083_at |
| 847 | FBgn0000261 | CG6871 | 0 | 66 | 143093_at |
| 54205 | FBgn0000409 | CG17903 | 1E−45 | 78 | 143115_at |
| 5966 | FBgn0000462 | CG6667 | 1E−72 | 46 | 143125_at |
| 5613 | FBgn0000489 | CG6117 | 1E−124 | 62 | 143130_at |
| 2128 | FBgn0000606 | CG2328 | 7E−39 | 42 | 143153_at |
| 5500 | FBgn0000711 | CG2096 | 1E−178 | 91 | 143162_at |
| 2597 | FBgn0001092 | CG8893 | 1E−147 | 76 | 143175_at |
| 2752 | FBgn0001145 | CG1743 | 1E−150 | 65 | 143181_at |
| 3320 | FBgn0001233 | CG1242 | 0 | 78 | 143198_at |
| 4212 | FBgn0001235 | CG17117 | 1E−111 | 57 | 143199_at |
| 7023 | FBgn0001994 | CG7664 | 3E−27 | 40 | 143223_at |
| 4001 | FBgn0002525 | CG6944 | 1E−111 | 36 | 143231_at |
| 6176 | FBgn0002593 | CG4087 | 2E−34 | 61 | 143247_at |
| 6161 | FBgn0002626 | CG7939 | 6E−58 | 77 | 143250_at |
| 5105 | FBgn0003067 | CG17725 | 0 | 68 | 143299_at |
| 5187 | FBgn0003068 | CG2647 | 5E−29 | 25 | 143300_at |
| 5501 | FBgn0003134 | CG6593 | 1E−172 | 88 | 143310_at |
| 3845 | FBgn0003205 | CG9375 | 2E−82 | 79 | 143317_at |
| 94233 | FBgn0003248 | CG16740 | 2E−53 | 38 | 143320_at |
| 6181 | FBgn0003274 | CG4918 | 3E−32 | 60 | 143326_at |
| 6647 | FBgn0003462 | CG11793 | 3E−50 | 61 | 143358_at |
| 1959 | FBgn0003499 | CG7847 | 4E−63 | 42 | 143365_at |
| 3921 | FBgn0003517 | CG14792 | 2E−94 | 61 | 143369_at |
| 6950 | FBgn0003676 | CG5374 | 0 | 73 | 143374_at |
| 4297 | FBgn0003862 | CG8651 | 5E−98 | 23 | 143384_at |
| 7280 | FBgn0003887 | CG9277 | 0 | 97 | 143390_at |
| 7280 | FBgn0003888 | CG3401 | 0 | 89 | 143392_r_at |
| 5793 | FBgn0004369 | CG2005 | 1E−161 | 46 | 143448_at |
| 11127 | FBgn0004380 | CG10642 | 0 | 58 | 143453_at |
| 6208 | FBgn0004404 | CG1527 | 3E−71 | 86 | 143461_at |
| 6231 | FBgn0004413 | CG10305 | 2E−47 | 82 | 143462_at |
| 5244 | FBgn0004513 | CG10181 | 0 | 43 | 143476_at |
| 6853 | FBgn0004575 | CG3985 | 1E−107 | 47 | 143485_at |
| 579 | FBgn0004862 | CG7902 | 2E−29 | 74 | 143523_at |
| 6218 | FBgn0005533 | CG3922 | 5E−52 | 81 | 143542_at |
| 811 | FBgn0005585 | CG9429 | 1E−163 | 66 | 143547_at |
| 5218 | FBgn0005640 | CG10579 | 1E−124 | 65 | 143565_at |
| 506 | FBgn0010217 | CG11154 | 0 | 89 | 143586_at |
| 166929 | FBgn0010383 | CG6816 | 1E−82 | 34 | 143606_at |
| 6142 | FBgn0010409 | CG6510 | 3E−68 | 68 | 143616_at |
| 6157 | FBgn0010410 | CG15442 | 4E−60 | 69 | 143617_at |
| 6222 | FBgn0010411 | CG8900 | 1E−69 | 77 | 143618_at |
| 6230 | FBgn0010413 | CG6684 | 4E−40 | 70 | 143620_at |
| 9296 | FBgn0010426 | CG8210 | 3E−47 | 72 | 143625_at |
| 6742 | FBgn0010438 | CG4337 | 3E−25 | 41 | 143628_at |
| 10952 | FBgn0010638 | CG10130 | 7E−31 | 67 | 143631_at |
| 1730 | FBgn0011202 | CG1768 | 0 | 40 | 143636_at |
| 6137 | FBgn0011272 | CG4651 | 1E−68 | 63 | 143642_at |
| 6261 | FBgn0011286 | CG10844 | 0 | 50 | 143650_at |
| 11140 | FBgn0011573 | CG12019 | 1E−105 | 51 | 143662_at |
| 5888 | FBgn0011700 | CG7948 | 1E−123 | 71 | 143679_at |
| 3385 | FBgn0011769 | CG4205 | 6E−46 | 61 | 143690_at |
| 81888 | FBgn0011770 | CG2227 | 8E−46 | 35 | 143691_at |
| 54433 | FBgn0011824 | CG4038 | 4E−70 | 59 | 143696_at |
| 1781 | FBgn0013761 | CG18000 | 0 | 50 | 143720_at |
| 56171 | FBgn0013810 | CG5526 | 0 | 59 | 143725_at |
| 8294 | FBgn0013981 | CG3379 | 4E−54 | 99 | 143733_at |
| 9261 | FBgn0013987 | CG3086 | 1E−132 | 63 | 143735_at |
| 4638 | FBgn0013988 | CG18255 | 1E−119 | 41 | 143736_at |
| 5034 | FBgn0014002 | CG6988 | 1E−155 | 60 | 143739_at |
| 4217 | FBgn0014006 | CG4720 | 0 | 41 | 143740_at |
| 6130 | FBgn0014026 | CG3314 | 1E−102 | 68 | 143746_at |
| 1820 | FBgn0004795 | CG5403 | 8E−59 | 43 | 143753_at |
| 3020 | FBgn0014857 | CG5825 | 7E−72 | 100 | 143766_i_at |
| 5001 | FBgn0015271 | CG7833 | 6E−71 | 35 | 143787_at |
| 5291 | FBgn0015279 | CG4141 | 0 | 37 | 143789_at |
| 5898 | FBgn0015286 | CG2849 | 2E−83 | 79 | 143790_at |
| 283455 | FBgn0015402 | CG2899 | 2E−87 | 50 | 143807_at |
| 6227 | FBgn0015521 | CG2986 | 2E−30 | 73 | 143812_at |
| 63973 | FBgn0015550 | CG7659 | 7E−20 | 37 | 143817_at |
| 324 | FBgn0015589 | CG1451 | 1E−133 | 53 | 143828_at |
| 11080 | FBgn0015657 | CG10578 | 1E−102 | 55 | 143835_at |
| 6133 | FBgn0015756 | CG6141 | 1E−66 | 65 | 143838_at |
| 8766 | FBgn0015790 | CG5771 | 1E−101 | 85 | 143843_at |
| 6198 | FBgn0015806 | CG10539 | 1E−158 | 72 | 143848_at |
| 358 | FBgn0015872 | CG9023 | 5E−50 | 41 | 143854_at |
| 122786 | FBgn0004583 | CG4114 | 2E−17 | 33 | 143858_at |
| 10419 | FBgn0015925 | CG3730 | 1E−120 | 40 | 143859_at |
| 1282 | FBgn0016075 | CG16858 | 0 | 42 | 143868_at |
| 522 | FBgn0016119 | CG4412 | 0.000000000000007 | 40 | 143870_at |
| 10476 | FBgn0016120 | CG6030 | 2E−36 | 45 | 143871_at |
| 3628 | FBgn0016672 | CG3028 | 2E−65 | 38 | 143875_at |
| 6159 | FBgn0016726 | CG10071 | 0.00000000000007 | 46 | 143880_at |
| 6014 | FBgn0017549 | CG8418 | 4E−65 | 66 | 143893_at |
| 9045 | FBgn0017579 | CG6253 | 7E−30 | 42 | 143898_at |
| 9377 | FBgn0019624 | CG14724 | 6E−31 | 52 | 143899_at |
| 6224 | FBgn0019936 | CG15693 | 5E−50 | 81 | 143904_at |
| 868 | FBgn0020224 | CG7037 | 1E−178 | 72 | 143910_at |
| 509 | FBgn0020235 | CG7610 | 2E−85 | 58 | 143911_at |
| 10417 | FBgn0020269 | CG10145 | 2E−43 | 34 | 143915_at |
| 2678 | FBgn0023167 | CG8427 | 3E−49 | 80 | 143973_at |
| 23312 | FBgn0023458 | CG3585 | 0 | 38 | 143986_at |
| 58508 | FBgn0023518 | CG3848 | 1E−173 | 53 | 143994_at |
| 83657 | FBgn0024196 | CG10751 | 3E−38 | 75 | 144010_at |
| 9611 | FBgn0024308 | CG4013 | 6E−73 | 30 | 144015_at |
| 6132 | FBgn0024939 | CG1263 | 1E−119 | 80 | 144032_at |
| 48 | FBgn0024957 | CG6342 | 0 | 67 | 144037_at |
| 7355 | FBgn0024994 | CG2675 | 9E−84 | 51 | 144045_at |
| 10425 | FBgn0025186 | CG5709 | 1E−165 | 53 | 144051_at |
| 8804 | FBgn0025456 | CG5413 | 2E−23 | 34 | 144063_at |
| 64080 | FBgn0025640 | CG13369 | 8E−64 | 44 | 144081_at |
| 4088 | FBgn0025800 | CG2262 | 1E−165 | 61 | 144094_at |
| 4257 | FBgn0025814 | CG1742 | 4E−27 | 45 | 144097_at |
| 10733 | FBgn0026371 | CG7186 | 3E−99 | 56 | 144123_at |
| 5728 | FBgn0026379 | CG5671 | 9E−77 | 44 | 144125_at |
| 6507 | FBgn0026439 | CG3747 | 1E−111 | 45 | 144146_at |
| 8404 | FBgn0026562 | CG6378 | 5E−28 | 31 | 144147_at |
| 10273 | FBgn0027052 | CG5203 | 2E−91 | 57 | 144169_at |
| 26521 | FBgn0027359 | CG1728 | 7E−23 | 58 | 144179_at |
| 23493 | FBgn0027788 | CG11194 | 7E−42 | 45 | 144192_at |
| 9567 | FBgn0027836 | CG5729 | 0 | 71 | 144194_at |
| 57122 | FBgn0027868 | CG6743 | 1E−131 | 33 | 144196_at |
| 10247 | FBgn0028510 | CG15261 | 4E−34 | 46 | 144220_at |
| 1992 | FBgn0028983 | CG10913 | 2E−61 | 36 | 144243_at |
| 1787 | FBgn0028707 | CG10692 | 8E−62 | 34 | 144249_at |
| 57019 | FBgn0001977 | CG4180 | 2E−45 | 38 | 144311_at |
| 1112 | FBgn0029504 | CG12690 | 2E−31 | 36 | 144337_at |
| 26580 | FBgn0040336 | CG9904 | 1E−50 | 40 | 144412_at |
| 55761 | FBgn0029713 | CG11436 | 4E−18 | 21 | 144481_at |
| 10436 | FBgn0029714 | CG3527 | 4E−76 | 63 | 144482_at |
| 58516 | FBgn0029738 | CG4068 | 6E−38 | 34 | 144501_at |
| 51337 | FBgn0029838 | CG4666 | 5E−27 | 35 | 144572_at |
| 10785 | FBgn0029857 | CG15897 | 2E−31 | 31 | 144588_at |
| 4714 | FBgn0029888 | CG3192 | 6E−22 | 38 | 144608_at |
| 2271 | FBgn0029889 | CG4095 | 0 | 70 | 144609_at |
| 223082 | FBgn0029911 | CG14435 | 3E−31 | 34 | 144622_at |
| 51144 | FBgn0029975 | CG1444 | 2E−68 | 54 | 144664_at |
| 2729 | FBgn0040319 | CG2259 | 0 | 59 | 144677_at |
| 3074 | FBgn0041629 | CG1787 | 2E−62 | 34 | 144712_at |
| 513 | FBgn0030184 | CG2968 | 6E−33 | 48 | 144787_at |
| 25796 | FBgn0030239 | CG17333 | 3E−46 | 43 | 144831_at |
| 114771 | FBgn0030310 | CG11709 | 5E−38 | 44 | 144882_at |
| 55836 | FBgn0030311 | CG11699 | 6E−16 | 34 | 144883_at |
| 79154 | FBgn0030332 | CG9360 | 2E−47 | 40 | 144893_at |
| 23423 | FBgn0030341 | CG1967 | 1E−49 | 50 | 144899_at |
| 124801 | FBgn0030364 | CG15735 | 2E−21 | 33 | 144912_at |
| 11073 | FBgn0002878 | CG11156 | 8E−67 | 24 | 145028_at |
| 83877 | FBgn0030522 | CG11103 | 6E−56 | 56 | 145032_at |
| 6898 | FBgn0030558 | CG1461 | 1E−127 | 51 | 145058_at |
| 4713 | FBgn0030605 | CG5548 | 6E−28 | 54 | 145097_at |
| 51228 | FBgn0030641 | CG6299 | 5E−17 | 31 | 145121_at |
| 7381 | FBgn0030733 | CG3560 | 2E−33 | 62 | 145177_at |
| 51084 | FBgn0030737 | CG9914 | 1E−79 | 50 | 145178_at |
| 7327 | FBgn0015374 | CG4443 | 3E−78 | 81 | 145193_at |
| 10094 | FBgn0030818 | CG8936 | 2E−61 | 60 | 145228_at |
| 2267 | FBgn0030880 | CG6788 | 8E−42 | 42 | 145267_at |
| 23413 | FBgn0030897 | CG5744 | 3E−75 | 71 | 145279_at |
| 51181 | FBgn0030968 | CG7322 | 8E−55 | 49 | 145332_at |
| 51207 | FBgn0030976 | CG7378 | 4E−34 | 47 | 145337_at |
| 2671 | FBgn0031068 | CG12534 | 2E−35 | 43 | 145394_at |
| 5825 | FBgn0031069 | CG12703 | 0 | 59 | 145395_at |
| 9325 | FBgn0031115 | CG11710 | 2E−84 | 39 | 145428_at |
| 21 | FBgn0031170 | CG1718 | 0 | 37 | 145471_at |
| 3960 | FBgn0031213 | CG11372 | 4E−29 | 30 | 145507_at |
| 55773 | FBgn0031304 | CG4552 | 1E−162 | 45 | 145558_at |
| 8241 | FBgn0031318 | CG4887 | 3E−99 | 30 | 145565_at |
| 25822 | FBgn0031322 | CG5001 | 1E−104 | 53 | 145566_at |
| 7386 | FBgn0021906 | CG7361 | 5E−77 | 68 | 145592_at |
| 10577 | FBgn0031381 | CG7291 | 5E−24 | 37 | 145612_at |
| 25901 | FBgn0031395 | CG10874 | 4E−18 | 36 | 145623_at |
| 27294 | FBgn0031418 | CG3609 | 2E−68 | 42 | 145637_at |
| 55967 | FBgn0031436 | CG3214 | 4E−27 | 42 | 145650_at |
| 7866 | FBgn0051694 | CG31694 | 2E−66 | 36 | 145680_at |
| 130617 | FBgn0031535 | CG12795 | 4E−47 | 41 | 145713_at |
| 51659 | FBgn0031599 | CG18013 | 5E−45 | 45 | 145755_at |
| 64747 | FBgn0031636 | CG12194 | 1E−142 | 54 | 145783_at |
| 8780 | FBgn0031643 | CG3008 | 1E−106 | 42 | 145788_at |
| 9526 | FBgn0031662 | CG3792 | 9E−62 | 48 | 145801_at |
| 6168 | FBgn0028696 | CG5827 | 1E−38 | 76 | 145808_at |
| 6293 | FBgn0031710 | CG7371 | 0 | 50 | 145825_at |
| 1803 | FBgn0031741 | CG11034 | 1E−115 | 33 | 145847_at |
| 10093 | FBgn0031781 | CG5972 | 8E−75 | 82 | 145872_at |
| 9693 | FBgn0031798 | CG9491 | 1E−128 | 52 | 145888_at |
| 5784 | FBgn0031799 | CG9493 | 1E−106 | 26 | 145889_at |
| 84844 | FBgn0031822 | CG9548 | 8E−63 | 96 | 145902_at |
| 135228 | FBgn0041182 | CG7052 | 1E−177 | 31 | 145971_at |
| 6166 | FBgn0031980 | CG7424 | 9E−44 | 76 | 146006_at |
| 4125 | FBgn0032069 | CG9468 | 0 | 40 | 146050_at |
| 967 | FBgn0032074 | CG9494 | 1E−33 | 32 | 146052_at |
| 51764 | FBgn0028433 | CG3694 | 0.000000000004 | 41 | 146078_at |
| 217 | FBgn0032114 | CG3752 | 0 | 71 | 146084_at |
| 1540 | FBgn0032210 | CG5603 | 1E−121 | 45 | 146137_at |
| 29970 | FBgn0032221 | CG5375 | 0.00000000004 | 24 | 146141_at |
| 64285 | FBgn0041723 | CG5364 | 1E−112 | 40 | 146149_at |
| 27079 | FBgn0051719 | CG31719 | 1E−118 | 47 | 146163_at |
| 3988 | FBgn0032264 | CG6113 | 8E−73 | 37 | 146165_at |
| 9354 | FBgn0010612 | CG6105 | 2E−25 | 54 | 146216_at |
| 219771 | FBgn0032378 | CG14939 | 1E−104 | 64 | 146253_at |
| 3652 | FBgn0032485 | CG9426 | 1E−109 | 37 | 146316_at |
| 80308 | FBgn0032522 | CG16848 | 1E−47 | 43 | 146337_at |
| 8649 | FBgn0032642 | CG5110 | 2E−28 | 45 | 146407_at |
| 56996 | FBgn0032689 | CG10413 | 0 | 42 | 146441_at |
| 64755 | FBgn0032700 | CG10338 | 1E−69 | 40 | 146445_at |
| 6156 | FBgn0015745 | CG10652 | 2E−46 | 81 | 146467_at |
| 54902 | FBgn0032744 | CG15173 | 8E−21 | 25 | 146470_at |
| 5829 | FBgn0051794 | CG31794 | 1E−140 | 76 | 146489_at |
| 6317 | FBgn0028986 | CG9334 | 5E−58 | 34 | 146555_at |
| 51202 | FBgn0032919 | CG9253 | 0 | 71 | 146573_at |
| 5274 | FBgn0028985 | CG9453 | 3E−61 | 35 | 146699_at |
| 55840 | FBgn0033166 | CG11166 | 8E−26 | 48 | 146736_at |
| 81577 | FBgn0033174 | CG11125 | 0.00000000002 | 31 | 146743_at |
| 57708 | FBgn0033183 | CG1620 | 1E−49 | 30 | 146745_at |
| 1528 | FBgn0033189 | CG2140 | 3E−30 | 48 | 146746_at |
| 54834 | FBgn0042135 | CG18812 | 1E−105 | 39 | 146764_at |
| 54855 | FBgn0050497 | CG30497 | 1E−110 | 61 | 146765_at |
| 63874 | FBgn0033226 | CG1882 | 2E−94 | 49 | 146771_at |
| 8993 | FBgn0043575 | CG14745 | 5E−41 | 47 | 146832_at |
| 60682 | FBgn0033349 | CG8243 | 6E−55 | 33 | 146846_at |
| 9939 | FBgn0033378 | CG8781 | 2E−56 | 63 | 146865_at |
| 84137 | FBgn0033380 | CG8069 | 6E−27 | 30 | 146867_at |
| 10567 | FBgn0033468 | CG1418 | 1E−24 | 36 | 146913_at |
| 7247 | FBgn0033528 | CG11761 | 8E−59 | 52 | 146953_at |
| 55505 | FBgn0033548 | CG7637 | 4E−20 | 66 | 146963_at |
| 4700 | FBgn0033570 | CG7712 | 1E−31 | 48 | 146973_at |
| 57599 | FBgn0033607 | CG9062 | 0 | 56 | 147005_at |
| 80218 | FBgn0024188 | CG12352 | 6E−70 | 70 | 147018_at |
| 8446 | FBgn0033647 | CG13197 | 3E−43 | 38 | 147032_at |
| 79648 | FBgn0033664 | CG8981 | 0.0000000000003 | 39 | 147046_at |
| 23480 | FBgn0033691 | CG8860 | 4E−29 | 86 | 147060_at |
| 8729 | FBgn0033714 | CG8487 | 0 | 45 | 147079_at |
| 2799 | FBgn0033836 | CG18278 | 1E−112 | 45 | 147151_at |
| 9145 | FBgn0033876 | CG10808 | 1E−43 | 40 | 147172_at |
| 444 | FBgn0034075 | CG8421 | 1E−109 | 35 | 147279_at |
| 6209 | FBgn0034138 | CG8332 | 2E−59 | 76 | 147317_at |
| 8662 | FBgn0034237 | CG4878 | 0 | 51 | 147373_at |
| 5105 | FBgn0034356 | CG10924 | 0 | 65 | 147444_at |
| 1036 | FBgn0034364 | CG5493 | 4E−56 | 52 | 147450_at |
| 2052 | FBgn0034405 | CG15102 | 1E−95 | 42 | 147472_at |
| 26090 | FBgn0034419 | CG15111 | 2E−64 | 37 | 147479_at |
| 8491 | FBgn0034421 | CG7097 | 1E−138 | 75 | 147480_at |
| 135138 | FBgn0034454 | CG15120 | 5E−44 | 57 | 147502_at |
| 10914 | FBgn0015949 | CG9854 | 0 | 52 | 147511_at |
| 25828 | FBgn0034472 | CG8517 | 9E−28 | 48 | 147518_at |
| 80344 | FBgn0034527 | CG9945 | 3E−82 | 37 | 147558_at |
| 10559 | FBgn0034532 | CG13436 | 1E−20 | 21 | 147559_at |
| 84191 | FBgn0050152 | CG30152 | 5E−39 | 57 | 147569_at |
| 11157 | FBgn0034564 | CG9344 | 1E−33 | 85 | 147588_at |
| 126328 | FBgn0034576 | CG9350 | 8E−16 | 35 | 147595_at |
| 6217 | FBgn0034743 | CG4046 | 2E−69 | 88 | 147686_at |
| 79643 | FBgn0034744 | CG4071 | 6E−21 | 41 | 147687_at |
| 5479 | FBgn0034753 | CG2852 | 3E−76 | 73 | 147691_at |
| 10588 | FBgn0034851 | CG11079 | 2E−32 | 35 | 147768_at |
| 1984 | FBgn0034967 | CG3186 | 2E−57 | 68 | 147834_at |
| 6136 | FBgn0002611 | CG3195 | 6E−75 | 79 | 147835_at |
| 79657 | FBgn0015544 | CG13570 | 8E−31 | 25 | 147842_at |
| 4702 | FBgn0035046 | CG3683 | 6E−38 | 46 | 147881_at |
| 4323 | FBgn0035049 | CG4859 | 1E−92 | 38 | 147882_at |
| 79020 | FBgn0035163 | CG13886 | 6E−55 | 34 | 147941_at |
| 51691 | FBgn0035271 | CG2021 | 2E−34 | 69 | 148002_at |
| 1130 | FBgn0035296 | CG11814 | 1E−126 | 36 | 148016_at |
| 6150 | FBgn0035335 | CG1320 | 4E−28 | 43 | 148041_at |
| 50488 | FBgn0010909 | CG16973 | 1E−178 | 46 | 148051_at |
| 219670 | FBgn0035354 | CG16984 | 3E−22 | 31 | 148053_at |
| 55856 | FBgn0035356 | CG16986 | 1E−21 | 39 | 148055_at |
| 3423 | FBgn0035445 | CG12014 | 1E−118 | 44 | 148109_at |
| 9669 | FBgn0026259 | CG10840 | 0 | 65 | 148111_r_at |
| 51072 | FBgn0035491 | CG11591 | 0.0000000000005 | 39 | 148143_at |
| 755 | FBgn0035497 | CG14995 | 3E−37 | 53 | 148145_at |
| 129831 | FBgn0035526 | CG1316 | 8E−74 | 35 | 148165_at |
| 2230 | FBgn0035529 | CG1319 | 4E−35 | 57 | 148166_at |
| 25809 | FBgn0052238 | CG32238 | 1E−166 | 67 | 148193_at |
| 23658 | FBgn0035675 | CG6610 | 5E−36 | 85 | 148261_at |
| 130589 | FBgn0035679 | CG10467 | 7E−75 | 44 | 148265_at |
| 55245 | FBgn0035722 | CG10075 | 5E−36 | 37 | 148291_at |
| 1628 | FBgn0035753 | CG8615 | 3E−71 | 69 | 148318_at |
| 142891 | FBgn0052380 | CG32380 | 1E−108 | 67 | 148329_at |
| 2052 | FBgn0035827 | CG8268 | 0.00000000000004 | 41 | 148367_at |
| 23144 | FBgn0035900 | CG6694 | 1E−32 | 28 | 148406_at |
| 6059 | FBgn0035946 | CG5651 | 0 | 77 | 148436_at |
| 64699 | FBgn0023479 | CG4821 | 1E−44 | 36 | 148446_at |
| 51151 | FBgn0035968 | CG4484 | 1E−73 | 30 | 148450_at |
| 5631 | FBgn0036030 | CG6767 | 1E−161 | 88 | 148484_at |
| 51571 | FBgn0052066 | CG32066 | 1E−92 | 54 | 148527_at |
| 4698 | FBgn0036100 | CG6463 | 1E−28 | 50 | 148532_at |
| 51647 | FBgn0036107 | CG7949 | 3E−59 | 71 | 148536_at |
| 51069 | FBgn0036135 | CG7636 | 4E−36 | 54 | 148557_at |
| 4524 | FBgn0036157 | CG7560 | 4E−21 | 27 | 148573_at |
| 23464 | FBgn0036208 | CG10361 | 1E−152 | 66 | 148605_at |
| 4711 | FBgn0011455 | CG9762 | 9E−31 | 49 | 148634_at |
| 51386 | FBgn0036258 | CG5642 | 1E−169 | 52 | 148635_at |
| 55586 | FBgn0036262 | CG6910 | 7E−88 | 56 | 148639_at |
| 203 | FBgn0022709 | CG17146 | 4E−46 | 48 | 148641_at |
| 210 | FBgn0036271 | CG10335 | 1E−109 | 61 | 148647_at |
| 6611 | FBgn0036272 | CG4300 | 3E−76 | 43 | 148648_at |
| 11065 | FBgn0027936 | CG10682 | 4E−53 | 60 | 148664_at |
| 51364 | FBgn0036338 | CG11253 | 2E−65 | 31 | 148692_at |
| 28988 | FBgn0036372 | CG10083 | 5E−62 | 30 | 148717_at |
| 55578 | FBgn0036374 | CG17689 | 8E−32 | 28 | 148719_at |
| 2029 | FBgn0036401 | CG6513 | 2E−19 | 45 | 148739_at |
| 5507 | FBgn0036428 | CG9238 | 2E−35 | 42 | 148753_at |
| 81550 | FBgn0036450 | CG13472 | 5E−47 | 31 | 148768_at |
| 283375 | FBgn0036461 | CG10006 | 6E−50 | 36 | 148775_at |
| 286053 | FBgn0036730 | CG13732 | 0.00000000003 | 25 | 148959_at |
| 80153 | FBgn0036735 | CG6311 | 5E−48 | 32 | 148962_at |
| 977 | FBgn0036769 | CG5492 | 4E−30 | 28 | 148983_at |
| 79017 | FBgn0036787 | CG4306 | 2E−26 | 37 | 148995_at |
| 6154 | FBgn0036825 | CG6846 | 9E−60 | 72 | 149019_at |
| 10282 | FBgn0036855 | CG14084 | 1E−17 | 39 | 149035_at |
| 55324 | FBgn0036888 | CG9330 | 0 | 56 | 149061_at |
| 84669 | FBgn0036913 | CG8334 | 1E−101 | 47 | 149075_at |
| 10101 | FBgn0037011 | CG4858 | 1E−84 | 55 | 149138_at |
| 8661 | FBgn0037249 | CG9805 | 0 | 44 | 149279_at |
| 9827 | FBgn0037297 | CG1116 | 4E−57 | 36 | 149311_at |
| 51097 | FBgn0037298 | CG2604 | 1E−81 | 39 | 149312_at |
| 9451 | FBgn0037327 | CG2087 | 1E−86 | 35 | 149333_at |
| 6165 | FBgn0037328 | CG2099 | 2E−35 | 61 | 149334_at |
| 3141 | FBgn0037332 | CG14670 | 9E−70 | 31 | 149335_at |
| 152007 | CG31482 | CG31482 | 5E−31 | 50 | 149411_at |
| 9317 | FBgn0037683 | CG18473 | 1E−89 | 45 | 149552_s_at |
| 6164 | FBgn0037686 | CG9354 | 9E−30 | 58 | 149554_at |
| 4857 | FBgn0026188 | CG8144 | 3E−95 | 43 | 149559_at |
| 89782 | FBgn0002478 | CG3953 | 1E−136 | 42 | 149618_at |
| 2731 | FBgn0037801 | CG3999 | 0 | 64 | 149624_at |
| 50 | FBgn0037862 | CG4706 | 0 | 70 | 149664_at |
| 25819 | CG31299 | CG31299 | 3E−72 | 47 | 149672_at |
| 6391 | FBgn0037873 | CG6666 | 1E−25 | 41 | 149673_at |
| 25994 | FBgn0037890 | CG17734 | 7E−17 | 52 | 149681_at |
| 83874 | FBgn0037917 | CG5344 | 5E−85 | 45 | 149694_at |
| 83733 | FBgn0037969 | CG18347 | 3E−87 | 53 | 149719_at |
| 8464 | FBgn0037981 | CG3169 | 1E−21 | 31 | 149726_at |
| 54187 | FBgn0038045 | CG5232 | 9E−91 | 47 | 149775_at |
| 79157 | FBgn0038053 | CG18549 | 1E−101 | 43 | 149779_at |
| 3312 | FBgn0038059 | CG6489 | 0 | 76 | 149782_at |
| 27232 | FBgn0038074 | CG6188 | 1E−91 | 57 | 149794_at |
| 54788 | FBgn0038195 | CG3061 | 2E−81 | 42 | 149868_at |
| 94121 | FBgn0038263 | CG7343 | 3E−87 | 43 | 149919_at |
| 11235 | FBgn0038331 | CG5073 | 2E−46 | 48 | 149953_at |
| 80070 | FBgn0038341 | CG14869 | 1E−152 | 30 | 149959_at |
| 97 | FBgn0038363 | CG18505 | 4E−20 | 45 | 149974_at |
| 29985 | FBgn0038412 | CG6898 | 8E−27 | 28 | 150003_at |
| 8293 | FBgn0038421 | CG17931 | 0.000000000001 | 57 | 150009_at |
| 51573 | FBgn0038432 | CG14883 | 0.0000000000001 | 23 | 150018_at |
| 158 | FBgn0038467 | CG3590 | 0 | 67 | 150041_at |
| 64963 | FBgn0038474 | CG5184 | 7E−26 | 49 | 150046_at |
| 5831 | FBgn0038516 | CG5840 | 2E−59 | 50 | 150080_at |
| 4191 | FBgn0038587 | CG7998 | 1E−114 | 59 | 150121_at |
| 83942 | FBgn0038630 | CG14305 | 2E−64 | 46 | 150150_at |
| 51692 | FBgn0038636 | CG7698 | 0 | 67 | 150154_at |
| 54840 | FBgn0038704 | CG5316 | 1E−31 | 47 | 150195_at |
| 9818 | FBgn0038722 | CG7360 | 4E−49 | 29 | 150209_at |
| 10257 | FBgn0038740 | CG4562 | 0 | 45 | 150223_at |
| 35 | FBgn0038742 | CG4703 | 1E−160 | 69 | 150225_at |
| 5523 | FBgn0038744 | CG4733 | 1E−141 | 56 | 150227_at |
| 22933 | FBgn0038788 | CG5085 | 1E−96 | 49 | 150255_at |
| 55217 | FBgn0038795 | CG4335 | 6E−41 | 32 | 150261_at |
| 6727 | FBgn0038808 | CG5417 | 5E−16 | 37 | 150266_at |
| 51592 | FBgn0023097 | CG5206 | 4E−94 | 26 | 150269_at |
| 2197 | FBgn0038834 | CG15697 | 5E−32 | 55 | 150281_at |
| 81889 | FBgn0038858 | CG5793 | 3E−53 | 49 | 150297_at |
| 1175 | FBgn0043012 | CG6056 | 3E−76 | 95 | 150308_at |
| 11267 | FBgn0038908 | CG6637 | 2E−69 | 50 | 150331_at |
| 403 | FBgn0038916 | CG6560 | 5E−65 | 65 | 150337_at |
| 670 | FBgn0038974 | CG5377 | 3E−65 | 45 | 150370_at |
| 8626 | FBgn0039044 | CG10873 | 0.00000000002 | 26 | 150416_at |
| 26973 | FBgn0029503 | CG6198 | 3E−84 | 43 | 150490_at |
| 5441 | FBgn0039218 | CG13628 | 8E−34 | 92 | 150520_at |
| 11285 | FBgn0039258 | CG11780 | 7E−77 | 52 | 150544_at |
| 51441 | FBgn0039261 | CG6422 | 6E−78 | 41 | 150546_at |
| 9147 | FBgn0039281 | CG11847 | 0 | 45 | 150553_at |
| 7436 | CG31092 | CG31092 | 0 | 42 | 150606_at |
| 1800 | FBgn0039420 | CG6154 | 3E−98 | 51 | 150654_at |
| 90522 | FBgn0039450 | CG5484 | 8E−66 | 43 | 150680_at |
| 9203 | FBgn0010328 | CG5965 | 1E−108 | 37 | 150721_at |
| 7468 | FBgn0039559 | CG4976 | 1E−133 | 31 | 150758_at |
| 7280 | FBgn0039567 | CG4869 | 0 | 73 | 150762_at |
| 1937 | FBgn0029176 | CG11901 | 1E−141 | 56 | 150815_at |
| 135 | FBgn0039747 | CG9753 | 7E−53 | 36 | 150872_at |
| 6128 | FBgn0039857 | CG11522 | 7E−58 | 47 | 150949_at |
| 4720 | FBgn0039909 | CG1970 | 0 | 76 | 150980_at |
| 6529 | FBgn0039915 | CG1732 | 0 | 61 | 150984_at |
| 29855 | FBgn0005596 | CG14513 | 2E−37 | 24 | 151107_r_at |
| 4695 | FBgn0040705 | CG15434 | 1E−17 | 50 | 151182_at |
| 10287 | FBgn0005613 | CG8404 | 7E−36 | 51 | 151227_f_at |
| 150678 | FBgn0040754 | CG17059 | 0.00000000001 | 52 | 151231_at |
| 153339 | FBgn0040890 | CG14199 | 1E−26 | 73 | 151363_at |
| 140838 | FBgn0029801 | CG15771 | 1E−31 | 41 | 151499_at |
| 1609 | CG31140 | CG31140 | 1E−131 | 36 | 151514_at |
| 5581 | FBgn0003093 | CG1954 | 0 | 57 | 151525_at |
| 1778 | FBgn0010349 | CG7507 | 0 | 73 | 151529_at |
| 6519 | FBgn0050359 | CG30359 | 1E−104 | 37 | 151539_at |
| 23428 | FBgn0039844 | CG1607 | 1E−141 | 49 | 151567_at |
| 23137 | FBgn0052438 | CG32438 | 4E−45 | 27 | 151585_at |
| 1655 | FBgn0035720 | CG10077 | 1E−177 | 56 | 151598_at |
| 25769 | FBgn0028704 | CG18660 | 1E−166 | 53 | 151600_at |
| 23046 | FBgn0032243 | CG5300 | 0 | 41 | 151611_at |
| 10422 | FBgn0028372 | CG11025 | 2E−29 | 28 | 151625_at |
| 5625 | FBgn0003423 | CG1417 | 1E−147 | 50 | 151627_s_at |
| 64083 | FBgn0031402 | CG7085 | 1E−117 | 70 | 151629_at |
| 6450 | FBgn0035772 | CG8582 | 2E−17 | 32 | 151636_at |
| 6780 | FBgn0003520 | CG5753 | 2E−63 | 31 | 151638_s_at |
| 5287 | FBgn0015278 | CG11621 | 0 | 37 | 151675_s_at |
| 64779 | FBgn0037245 | CG14648 | 2E−43 | 28 | 151683_at |
| 817 | FBgn0004624 | CG18069 | 0 | 77 | 151686_s_at |
| 28996 | FBgn0035142 | CG17090 | 0 | 51 | 151694_at |
| 7170 | FBgn0003721 | CG4898 | 3E−82 | 56 | 151700_at |
| 10904 | FBgn0040239 | CG4867 | 2E−22 | 62 | 151711_at |
| 84988 | FBgn0036801 | CG6896 | 9E−93 | 41 | 151721_at |
| 4907 | FBgn0050104 | CG30104 | 1E−111 | 40 | 151726_at |
| 7094 | FBgn0035910 | CG6831 | 0 | 47 | 151754_at |
| 4520 | FBgn0040305 | CG3743 | 5E−75 | 44 | 151761_at |
| 10797 | FBgn0010222 | CG18466 | 4E−93 | 57 | 151767_at |
| 9512 | FBgn0038271 | CG3731 | 0 | 70 | 151769_at |
| 11069 | FBgn0033102 | CG3427 | 0 | 50 | 151791_at |
| 57515 | FBgn0028399 | CG4672 | 1E−110 | 43 | 151794_at |
| 3417 | FBgn0013617 | CG7176 | 1E−177 | 72 | 151795_at |
| 2203 | FBgn0051692 | CG31692 | 1E−120 | 64 | 151797_at |
| 10527 | FBgn0026252 | CG7935 | 0 | 54 | 151799_at |
| 8722 | FBgn0037303 | CG12163 | 5E−75 | 45 | 151801_at |
| 5862 | FBgn0014009 | CG3269 | 1E−107 | 89 | 151803_at |
| 84263 | FBgn0039537 | CG5590 | 2E−53 | 43 | 151807_at |
| 55968 | FBgn0033179 | CG11139 | 7E−68 | 37 | 151810_at |
| 117283 | FBgn0034644 | CG10082 | 9E−45 | 48 | 151811_at |
| 10445 | FBgn0035489 | CG1135 | 1E−111 | 55 | 151816_at |
| 6576 | CG31305 | CG31305 | 2E−81 | 68 | 151826_at |
| 4942 | FBgn0036898 | CG8782 | 1E−179 | 70 | 151832_at |
| 9531 | FBgn0052130 | CG32130 | 3E−16 | 23 | 151834_at |
| 10418 | FBgn0033710 | CG17739 | 1E−109 | 31 | 151836_at |
| 706 | FBgn0031263 | CG2789 | 1E−36 | 47 | 151838_at |
| 7347 | FBgn0010288 | CG4265 | 1E−60 | 50 | 151849_at |
| 4125 | FBgn0027611 | CG6206 | 0 | 47 | 151851_at |
| 1468 | FBgn0027610 | CG8790 | 3E−85 | 58 | 151853_at |
| 54968 | FBgn0035805 | CG7506 | 3E−26 | 35 | 151858_at |
| 339 | CG31170 | CG31170 | 5E−88 | 77 | 151866_at |
| 6648 | FBgn0010213 | CG8905 | 1E−74 | 62 | 151875_at |
| 9777 | FBgn0028541 | CG7364 | 0 | 64 | 151878_at |
| 55572 | FBgn0033093 | CG3270 | 1E−107 | 49 | 151882_at |
| 2194 | FBgn0042627 | CG3524 | 0 | 45 | 151887_at |
| 23585 | FBgn0035528 | CG15012 | 3E−26 | 38 | 151896_at |
| 2730 | FBgn0039001 | CG4919 | 5E−27 | 30 | 151897_at |
| 23633 | FBgn0024889 | CG8548 | 0 | 62 | 151898_at |
| 3242 | FBgn0036992 | CG11796 | 1E−140 | 63 | 151899_at |
| 11162 | FBgn0030668 | CG8128 | 2E−34 | 38 | 151908_at |
| 26227 | FBgn0032350 | CG6287 | 2E−93 | 52 | 151909_at |
| 84466 | FBgn0027594 | CG2086 | 0 | 37 | 151918_s_at |
| 9351 | FBgn0010620 | CG10939 | 1E−19 | 40 | 151959_at |
| 4640 | FBgn0010246 | CG9155 | 0 | 49 | 151960_at |
| 275 | FBgn0032287 | CG6415 | 1E−100 | 52 | 151967_at |
| 51160 | FBgn0021814 | CG12770 | 3E−78 | 64 | 151970_at |
| 10989 | FBgn0019960 | CG6455 | 2E−62 | 37 | 151979_at |
| 7866 | FBgn0037747 | CG8481 | 2E−18 | 39 | 151982_at |
| 126326 | FBgn0034797 | CG12781 | 2E−31 | 34 | 151996_at |
| 57118 | FBgn0016126 | CG1495 | 1E−119 | 61 | 151997_at |
| 10048 | FBgn0033522 | CG11763 | 1E−144 | 49 | 152018_at |
| 3178 | FBgn0004237 | CG12749 | 1E−99 | 50 | 152021_at |
| 10550 | FBgn0032704 | CG10373 | 1E−28 | 36 | 152035_at |
| 54539 | FBgn0027785 | CG6008 | 0.00000000001 | 32 | 152036_at |
| 84064 | FBgn0032032 | CG17294 | 1E−60 | 49 | 152042_at |
| 57130 | FBgn0027582 | CG6230 | 0 | 55 | 152048_at |
| 91768 | FBgn0027581 | CG6191 | 5E−58 | 37 | 152052_at |
| 5091 | FBgn0027580 | CG1516 | 0 | 71 | 152059_at |
| 4677 | FBgn0028492 | CG10687 | 0 | 72 | 152066_at |
| 2639 | FBgn0031824 | CG9547 | 1E−163 | 68 | 152078_at |
| 11112 | FBgn0034390 | CG15093 | 1E−79 | 46 | 152083_at |
| 57678 | FBgn0027579 | CG5508 | 2E−82 | 30 | 152088_at |
| 9200 | FBgn0032394 | CG6746 | 5E−39 | 33 | 152090_at |
| 51239 | FBgn0051919 | CG31919 | 9E−36 | 45 | 152093_at |
| 59353 | FBgn0025712 | CG13920 | 1E−24 | 37 | 152097_at |
| 28970 | FBgn0035189 | CG9119 | 2E−76 | 47 | 152102_at |
| 51 | FBgn0027572 | CG5009 | 1E−151 | 43 | 152121_s_at |
| 2194 | FBgn0027571 | CG3523 | 0 | 48 | 152126_at |
| 10087 | FBgn0027569 | CG7207 | 1E−139 | 42 | 152129_at |
| 10113 | FBgn0031779 | CG9175 | 8E−54 | 31 | 152142_at |
| 36 | FBgn0036824 | CG3902 | 1E−141 | 62 | 152151_at |
| 26065 | FBgn0045828 | CG10686 | 2E−40 | 30 | 152168_at |
| 6723 | FBgn0037723 | CG8327 | 8E−93 | 55 | 152178_at |
| 5162 | FBgn0039635 | CG11876 | 1E−135 | 67 | 152185_at |
| 9817 | FBgn0038475 | CG3962 | 1E−160 | 49 | 152189_at |
| 10745 | FBgn0028579 | CG3268 | 2E−76 | 43 | 152190_at |
| 821 | FBgn0015622 | CG11958 | 1E−162 | 48 | 152192_at |
| 55033 | FBgn0010470 | CG9847 | 3E−45 | 45 | 152208_at |
| 4729 | FBgn0030853 | CG5703 | 2E−90 | 71 | 152209_at |
| 56267 | FBgn0037955 | CG6950 | 1E−129 | 54 | 152211_at |
| 80224 | FBgn0032986 | CG3262 | 3E−63 | 44 | 152214_at |
| 9990 | FBgn0025698 | CG5594 | 0 | 58 | 152231_at |
| 10352 | FBgn0036763 | CG7441 | 1E−92 | 52 | 152250_at |
| 6557 | CG31547 | CG31547 | 0 | 41 | 152262_at |
| 149465 | FBgn0034745 | CG4329 | 2E−60 | 27 | 152270_at |
| 25873 | FBgn0002579 | CG7622 | 2E−33 | 62 | 152271_at |
| 6506 | FBgn0026438 | CG3159 | 1E−121 | 42 | 152272_at |
| 2108 | FBgn0010516 | CG8996 | 1E−123 | 70 | 152276_at |
| 9851 | FBgn0003218 | CG11111 | 0 | 45 | 152278_at |
| 829 | FBgn0034577 | CG10540 | 1E−105 | 64 | 152284_at |
| 8721 | FBgn0026208 | CG4143 | 1E−49 | 65 | 152287_at |
| 6009 | FBgn0041191 | CG1081 | 2E−62 | 63 | 152298_at |
| 79031 | FBgn0036237 | CG18593 | 8E−65 | 49 | 152300_at |
| 8803 | FBgn0037643 | CG11963 | 1E−140 | 59 | 152304_at |
| 2820 | FBgn0034048 | CG8256 | 0 | 63 | 152312_at |
| 1777 | FBgn0000477 | CG7780 | 3E−46 | 32 | 152336_at |
| 2585 | FBgn0035950 | CG5288 | 5E−95 | 43 | 152351_at |
| 79989 | FBgn0038358 | CG4525 | 1E−166 | 52 | 152353_at |
| 26235 | FBgn0030555 | CG1839 | 3E−78 | 32 | 152361_at |
| 5222 | FBgn0032049 | CG13095 | 2E−87 | 43 | 152369_at |
| 1152 | FBgn0052031 | CG32031 | 9E−76 | 44 | 152383_at |
| 793 | FBgn0004580 | CG6702 | 2E−58 | 46 | 152384_at |
| 4666 | FBgn0017565 | CG8759 | 9E−60 | 61 | 152402_at |
| 31 | FBgn0033246 | CG11198 | 0 | 63 | 152407_at |
| 9772 | FBgn0022153 | CG3054 | 1E−64 | 36 | 152413_at |
| 5702 | FBgn0028684 | CG10370 | 0 | 86 | 152417_at |
| 2326 | FBgn0033079 | CG3174 | 2E−33 | 36 | 152425_at |
| 1727 | FBgn0036211 | CG5946 | 4E−94 | 62 | 152426_at |
| 83723 | FBgn0028480 | CG17841 | 2E−25 | 30 | 152431_at |
| 56241 | CG31004 | CG31004 | 5E−80 | 30 | 152434_at |
| 84067 | FBgn0025681 | CG3558 | 9E−95 | 41 | 152450_at |
| 5832 | FBgn0037146 | CG7470 | 0 | 58 | 152455_at |
| 23636 | FBgn0034118 | CG6251 | 2E−64 | 37 | 152469_at |
| 55277 | FBgn0035484 | CG11594 | 1E−152 | 49 | 152482_at |
| 83861 | FBgn0052392 | CG32392 | 7E−54 | 39 | 152495_at |
| 90 | FBgn0003317 | CG1891 | 1E−143 | 54 | 152507_at |
| 80148 | FBgn0031897 | CG13784 | 1E−31 | 58 | 152508_at |
| 11181 | FBgn0003748 | CG9364 | 1E−140 | 44 | 152512_at |
| 9819 | FBgn0010460 | CG5461 | 1E−41 | 26 | 152525_at |
| 4953 | FBgn0013307 | CG8721 | 3E−87 | 46 | 152531_at |
| 6311 | FBgn0038338 | CG5166 | 2E−54 | 23 | 152532_at |
| 6389 | FBgn0017539 | CG17246 | 0 | 73 | 152538_at |
| 128866 | FBgn0033385 | CG8055 | 5E−64 | 57 | 152547_at |
| 5547 | FBgn0032864 | CG2493 | 1E−116 | 46 | 152568_at |
| 54363 | FBgn0050019 | CG30019 | 2E−98 | 48 | 152569_at |
| 8991 | FBgn0038115 | CG7966 | 1E−140 | 51 | 152571_at |
| 11319 | FBgn0035324 | CG5714 | 2E−41 | 31 | 152574_at |
| 2109 | FBgn0039697 | CG7834 | 1E−94 | 70 | 152584_at |
| 55858 | FBgn0038297 | CG4196 | 3E−73 | 60 | 152602_at |
| 3295 | FBgn0030731 | CG3415 | 1E−175 | 52 | 152603_at |
| 140690 | FBgn0035769 | CG8591 | 4E−88 | 41 | 152607_at |
| 132 | FBgn0036337 | CG11255 | 5E−97 | 52 | 152613_at |
| 9293 | FBgn0035538 | CG18314 | 2E−25 | 26 | 152630_at |
| 4659 | FBgn0052156 | CG32156 | 1E−120 | 30 | 152655_at |
| 4522 | FBgn0020385 | CG4067 | 0 | 61 | 152669_at |
| 2952 | FBgn0030484 | CG1681 | 1E−33 | 38 | 152675_at |
| 9374 | FBgn0032358 | CG4851 | 3E−59 | 40 | 152684_at |
| 5207 | FBgn0027621 | CG3400 | 1E−153 | 61 | 152692_at |
| 22884 | FBgn0038617 | CG12333 | 1E−120 | 50 | 152706_at |
| 7450 | FBgn0029167 | CG7002 | 1E−168 | 30 | 152708_at |
| 5471 | FBgn0041194 | CG10078 | 1E−177 | 61 | 152709_at |
| 10973 | FBgn0038344 | CG5205 | 0 | 58 | 152730_at |
| 7353 | FBgn0036136 | CG6233 | 6E−93 | 55 | 152735_at |
| 22872 | FBgn0033339 | CG8266 | 0 | 34 | 152754_at |
| 23175 | FBgn0033269 | CG8709 | 1E−130 | 33 | 152755_at |
| 5226 | FBgn0004654 | CG3724 | 0 | 71 | 152781_at |
| 51110 | FBgn0031987 | CG12375 | 6E−68 | 45 | 152792_at |
| 2806 | FBgn0031380 | CG4233 | 1E−155 | 64 | 152801_at |
| 22 | FBgn0035244 | CG7955 | 0 | 58 | 152811_at |
| 9390 | FBgn0019952 | CG6331 | 1E−92 | 35 | 152843_at |
| 54764 | FBgn0037734 | CG9448 | 1E−157 | 42 | 152844_at |
| 166012 | FBgn0051743 | CG31743 | 7E−52 | 41 | 152851_at |
| 79823 | FBgn0032857 | CG10947 | 2E−27 | 42 | 152852_at |
| 81034 | FBgn0033391 | CG8026 | 5E−92 | 53 | 152861_at |
| 2744 | FBgn0033765 | CG8772 | 1E−169 | 54 | 152880_at |
| 9371 | FBgn0004381 | CG7293 | 1E−175 | 45 | 152894_at |
| 7111 | FBgn0011716 | CG1539 | 2E−61 | 36 | 152898_at |
| 84947 | FBgn0032699 | CG10383 | 4E−41 | 37 | 152901_at |
| 51011 | FBgn0038924 | CG6028 | 9E−68 | 54 | 152903_at |
| 6319 | FBgn0043044 | CG5887 | 1E−102 | 58 | 152911_at |
| 9382 | FBgn0037998 | CG4848 | 1E−49 | 24 | 152916_at |
| 8525 | FBgn0003217 | CG10966 | 0 | 41 | 152920_at |
| 26960 | FBgn0003244 | CG6775 | 0 | 67 | 152922_at |
| 840 | FBgn0019972 | CG7788 | 8E−55 | 43 | 152941_at |
| 7317 | FBgn0023143 | CG1782 | 0 | 59 | 152946_at |
| 5579 | FBgn0004784 | CG6518 | 0 | 53 | 152951_at |
| 5876 | FBgn0028970 | CG18627 | 1E−123 | 64 | 152961_at |
| 3284 | FBgn0036698 | CG7724 | 5E−29 | 28 | 152966_at |
| 221 | FBgn0010548 | CG11140 | 1E−135 | 50 | 152970_at |
| 175 | FBgn0033431 | CG1827 | 1E−93 | 55 | 152972_at |
| 590 | FBgn0000024 | CG17907 | 1E−110 | 36 | 152974_at |
| 28962 | FBgn0035440 | CG14969 | 4E−20 | 26 | 152988_at |
| 622 | FBgn0033679 | CG8888 | 2E−42 | 33 | 152990_at |
| 7078 | FBgn0025879 | CG6281 | 0.0000000000003 | 26 | 153002_at |
| 90864 | FBgn0040280 | CG14041 | 1E−32 | 35 | 153005_at |
| 3420 | FBgn0038922 | CG6439 | 1E−119 | 60 | 153008_at |
| 5799 | FBgn0051795 | CG31795 | 1E−134 | 51 | 153019_at |
| 5564 | FBgn0033383 | CG8057 | 2E−68 | 62 | 153026_at |
| 6559 | FBgn0036279 | CG4357 | 0 | 47 | 153037_at |
| 189 | FBgn0014031 | CG3926 | 1E−95 | 45 | 153046_at |
| 64374 | FBgn0039296 | CG10420 | 4E−33 | 30 | 153052_at |
| 22905 | FBgn0028582 | CG8532 | 1E−104 | 38 | 153065_at |
| 64699 | FBgn0023479 | CG4821 | 1E−44 | 36 | 153070_at |
| 905 | FBgn0025455 | CG6292 | 5E−90 | 34 | 153091_at |
| 8407 | FBgn0035499 | CG14996 | 1E−32 | 42 | 153092_at |
| 64422 | FBgn0036813 | CG6877 | 1E−119 | 62 | 153099_at |
| 84928 | FBgn0036710 | CG6479 | 2E−46 | 27 | 153108_at |
| 4245 | FBgn0034521 | CG13431 | 1E−126 | 53 | 153109_at |
| 1743 | FBgn0037891 | CG5214 | 1E−133 | 62 | 153115_at |
| 23646 | FBgn0032923 | CG9248 | 1E−103 | 48 | 153117_at |
| 4728 | FBgn0017567 | CG3944 | 1E−85 | 82 | 153134_at |
| 6139 | FBgn0029897 | CG3203 | 4E−72 | 70 | 153138_at |
| 29968 | FBgn0014427 | CG11899 | 1E−127 | 63 | 153150_at |
| 10190 | FBgn0037843 | CG4511 | 7E−65 | 53 | 153166_at |
| 4716 | FBgn0021967 | CG8844 | 2E−21 | 31 | 153168_at |
| 167227 | FBgn0036534 | CG6169 | 3E−25 | 46 | 153180_at |
| 5694 | FBgn0010590 | CG8392 | 1E−69 | 57 | 153183_at |
| 85365 | FBgn0035401 | CG1291 | 1E−111 | 51 | 153189_at |
| 10240 | FBgn0036557 | CG5904 | 6E−35 | 35 | 153207_at |
| 6147 | FBgn0026372 | CG7977 | 3E−52 | 62 | 153215_at |
| 64129 | FBgn0034709 | CG3074 | 1E−94 | 42 | 153218_at |
| 84895 | FBgn0030037 | CG12125 | 2E−74 | 33 | 153237_at |
| 3422 | FBgn0038876 | CG5919 | 1E−57 | 49 | 153240_at |
| 55854 | FBgn0033317 | CG8635 | 1E−115 | 53 | 153244_at |
| 55969 | FBgn0033209 | CG12107 | 7E−36 | 64 | 153246_at |
| 55669 | FBgn0029870 | CG3869 | 0 | 46 | 153256_at |
| 2762 | FBgn0031661 | CG8890 | 1E−150 | 70 | 153281_at |
| 54976 | FBgn0035896 | CG6983 | 0.000000000000008 | 30 | 153282_at |
| 515 | FBgn0019644 | CG8189 | 4E−49 | 46 | 153283_at |
| 51263 | FBgn0034089 | CG8446 | 3E−56 | 38 | 153289_at |
| 51477 | FBgn0025885 | CG11143 | 0 | 61 | 153296_at |
| 81614 | FBgn0032451 | CG12292 | 1E−96 | 51 | 153299_at |
| 4704 | FBgn0037001 | CG6020 | 7E−76 | 44 | 153309_at |
| 9410 | FBgn0031229 | CG3436 | 1E−121 | 61 | 153310_at |
| 3313 | FBgn0001220 | CG8542 | 0 | 77 | 153311_at |
| 23521 | FBgn0037351 | CG1475 | 3E−68 | 58 | 153316_at |
| 4848 | FBgn0017550 | CG2161 | 2E−66 | 51 | 153321_at |
| 4329 | FBgn0023537 | CG17896 | 0 | 68 | 153323_at |
| 3065 | FBgn0015805 | CG7471 | 0 | 77 | 153325_at |
| 4723 | FBgn0031771 | CG9140 | 0 | 85 | 153326_at |
| 11333 | FBgn0029715 | CG11444 | 5E−33 | 43 | 153333_at |
| 1615 | FBgn0002069 | CG3821 | 0 | 68 | 153336_at |
| 2819 | FBgn0001128 | CG9042 | 1E−128 | 66 | 153347_at |
| 1537 | FBgn0035600 | CG4769 | 3E−95 | 59 | 153348_at |
| 7905 | FBgn0033906 | CG8331 | 1E−45 | 52 | 153351_at |
| 51068 | FBgn0023542 | CG3460 | 1E−155 | 53 | 153358_at |
| 7001 | FBgn0040309 | CG1633 | 5E−77 | 71 | 153359_at |
| 1371 | FBgn0021944 | CG3433 | 1E−130 | 59 | 153362_at |
| 2592 | FBgn0031845 | CG9232 | 1E−119 | 57 | 153364_at |
| 7086 | FBgn0037607 | CG8036 | 0 | 59 | 153373_at |
| 9320 | FBgn0037277 | CG17735 | 0 | 54 | 153383_at |
| 79969 | FBgn0035989 | CG3967 | 3E−36 | 42 | 153386_at |
| 10228 | FBgn0037084 | CG7736 | 2E−57 | 41 | 153387_at |
| 10287 | FBgn0028544 | CG16884 | 0.0000000000008 | 33 | 153391_at |
| 9049 | FBgn0030345 | CG1847 | 2E−65 | 40 | 153402_at |
| 57727 | FBgn0024542 | CG8614 | 6E−25 | 30 | 153420_at |
| 528 | FBgn0020611 | CG8048 | 1E−146 | 65 | 153422_at |
| 10606 | FBgn0020513 | CG3989 | 1E−140 | 59 | 153428_at |
| 55034 | FBgn0002641 | CG1692 | 6E−95 | 42 | 153431_at |
| 1979 | FBgn0022073 | CG8846 | 5E−18 | 39 | 153432_at |
| 3248 | FBgn0011693 | CG4899 | 9E−36 | 36 | 153433_at |
| 5053 | FBgn0001208 | CG7399 | 1E−157 | 62 | 153434_at |
| 498 | FBgn0011211 | CG3612 | 0 | 81 | 153435_at |
| 8508 | FBgn0030724 | CG9212 | 5E−70 | 49 | 153450_at |
| 1020 | FBgn0013762 | CG8203 | 1E−134 | 79 | 153451_at |
| 10444 | FBgn0043458 | CG12084 | 1E−124 | 37 | 153456_at |
| 481 | FBgn0015776 | CG9258 | 2E−33 | 33 | 153468_at |
| 8027 | FBgn0027363 | CG6521 | 1E−104 | 39 | 153478_at |
| 6633 | FBgn0037434 | CG1249 | 4E−55 | 88 | 153483_at |
| 64771 | FBgn0030838 | CG5445 | 5E−26 | 37 | 153484_at |
| 11124 | FBgn0034068 | CG8400 | 1E−106 | 34 | 153493_at |
| 6396 | FBgn0024509 | CG6773 | 1E−106 | 58 | 153498_at |
| 56888 | FBgn0037655 | CG11984 | 6E−86 | 52 | 153500_at |
| 2079 | FBgn0011586 | CG1871 | 2E−44 | 79 | 153502_at |
| 9342 | FBgn0034913 | CG11173 | 5E−28 | 31 | 153505_at |
| 5530 | FBgn0011826 | CG9842 | 0 | 83 | 153523_at |
| 10981 | FBgn0015788 | CG8024 | 3E−79 | 67 | 153524_at |
| 158584 | FBgn0033717 | CG8839 | 1E−124 | 46 | 153538_at |
| 84376 | FBgn0001202 | CG10653 | 1E−109 | 33 | 153540_at |
| 56910 | FBgn0032513 | CG6565 | 2E−36 | 27 | 153542_at |
| 55082 | FBgn0051712 | CG31712 | 2E−56 | 47 | 153569_at |
| 231 | FBgn0036182 | CG6084 | 1E−106 | 59 | 153576_at |
| 1399 | FBgn0024811 | CG1587 | 4E−65 | 44 | 153622_at |
| 23291 | FBgn0023423 | CG3412 | 0 | 82 | 153633_at |
| 11143 | FBgn0031911 | CG5229 | 1E−136 | 44 | 153634_at |
| 1642 | FBgn0027049 | CG7769 | 0 | 62 | 153636_at |
| 7443 | FBgn0027889 | CG6386 | 2E−67 | 37 | 153644_at |
| 1968 | FBgn0003600 | CG6476 | 0 | 83 | 153645_at |
| 9779 | FBgn0038129 | CG8449 | 1E−72 | 38 | 153652_at |
| 9053 | FBgn0035500 | CG14998 | 2E−42 | 27 | 153658_at |
| 5165 | FBgn0017558 | CG8808 | 1E−123 | 58 | 153665_at |
| 8539 | FBgn0027885 | CG6582 | 1E−122 | 46 | 153667_at |
| 26127 | FBgn0031871 | CG10158 | 2E−25 | 32 | 153669_at |
| 51719 | FBgn0017572 | CG4083 | 1E−132 | 70 | 153674_at |
| 7913 | FBgn0010567 | CG5935 | 4E−29 | 42 | 153681_at |
| 8452 | FBgn0001980 | CG11861 | 0 | 69 | 153701_at |
| 10049 | FBgn0034091 | CG8448 | 2E−50 | 45 | 153707_at |
| 6342 | FBgn0015808 | CG17320 | 0 | 59 | 153713_at |
| 27089 | FBgn0039244 | CG11069 | 4E−58 | 30 | 153724_at |
| 51029 | FBgn0033551 | CG7222 | 1E−66 | 62 | 153730_at |
| 116987 | CG31811 | CG31811 | 0 | 49 | 153741_at |
| 5347 | FBgn0003124 | CG12306 | 1E−175 | 52 | 153742_at |
| 55632 | FBgn0005683 | CG5354 | 2E−46 | 34 | 153746_at |
| 2885 | FBgn0004638 | CG6033 | 2E−76 | 65 | 153747_at |
| 4779 | FBgn0000338 | CG17894 | 1E−39 | 36 | 153758_at |
| 10856 | FBgn0040075 | CG9750 | 0 | 78 | 153759_at |
| 55902 | FBgn0012034 | CG9390 | 0 | 66 | 153769_at |
| 10541 | FBgn0034282 | CG5784 | 4E−51 | 47 | 153773_at |
| 196463 | FBgn0016031 | CG10645 | 1E−32 | 31 | 153774_at |
| 7360 | FBgn0035978 | CG4347 | 1E−170 | 62 | 153782_at |
| 9441 | FBgn0039923 | CG1793 | 0.00000000000002 | 51 | 153799_at |
| 6628 | FBgn0010083 | CG5352 | 3E−67 | 62 | 153800_at |
| 255919 | FBgn0036090 | CG8009 | 4E−23 | 54 | 153807_at |
| 2580 | FBgn0037218 | CG1107 | 1E−147 | 41 | 153829_at |
| 285672 | FBgn0029873 | CG3918 | 8E−28 | 39 | 153833_at |
| 9406 | FBgn0034750 | CG3732 | 3E−58 | 45 | 153834_at |
| 116039 | FBgn0004893 | CG10021 | 4E−77 | 75 | 153835_at |
| 10231 | FBgn0020250 | CG6072 | 6E−39 | 43 | 153839_at |
| 8560 | FBgn0001941 | CG9078 | 1E−124 | 61 | 153842_at |
| 51166 | FBgn0036117 | CG6321 | 3E−18 | 23 | 153845_at |
| 51247 | FBgn0038100 | CG12358 | 0.00000000001 | 39 | 153850_at |
| 54715 | FBgn0052062 | CG32062 | 2E−41 | 65 | 153863_at |
| 396 | FBgn0036921 | CG7823 | 3E−52 | 51 | 153882_at |
| 863 | FBgn0005636 | CG3385 | 9E−64 | 30 | 153902_at |
| 55737 | FBgn0034708 | CG5625 | 0 | 59 | 153904_at |
| 10139 | FBgn0030088 | CG7039 | 1E−71 | 63 | 153905_at |
| 167153 | FBgn0030353 | CG15737 | 1E−55 | 39 | 153926_at |
| 9706 | FBgn0036313 | CG10967 | 1E−101 | 42 | 153938_at |
| 28514 | FBgn0000463 | CG3619 | 1E−161 | 48 | 153950_at |
| 7511 | FBgn0026150 | CG6291 | 1E−168 | 48 | 153980_at |
| 6229 | FBgn0034751 | CG3751 | 7E−55 | 77 | 153994_at |
| 84946 | FBgn0027525 | CG7686 | 1E−89 | 40 | 154018_at |
| 23534 | FBgn0031456 | CG2848 | 0 | 41 | 154021_at |
| 4673 | FBgn0015268 | CG5330 | 3E−92 | 45 | 154024_at |
| 3069 | FBgn0027835 | CG5170 | 0 | 46 | 154042_at |
| 23761 | FBgn0039143 | CG5991 | 7E−76 | 48 | 154048_at |
| 84219 | FBgn0027518 | CG7609 | 1E−146 | 41 | 154050_at |
| 6879 | FBgn0024909 | CG2670 | 2E−48 | 44 | 154051_at |
| 7296 | FBgn0020653 | CG2151 | 1E−149 | 55 | 154053_at |
| 79591 | FBgn0037638 | CG8379 | 1E−149 | 43 | 154054_at |
| 151742 | FBgn0027515 | CG7115 | 7E−56 | 57 | 154059_at |
| 24137 | FBgn0011606 | CG8590 | 1E−173 | 34 | 154060_at |
| 55299 | FBgn0052253 | CG32253 | 4E−88 | 48 | 154073_at |
| 23381 | FBgn0019890 | CG8954 | 4E−24 | 24 | 154083_at |
| 1491 | FBgn0000566 | CG5345 | 1E−140 | 63 | 154085_at |
| 56261 | FBgn0031566 | CG2818 | 1E−103 | 40 | 154114_at |
| 10946 | FBgn0014366 | CG2925 | 0 | 65 | 154122_at |
| 54778 | FBgn0037944 | CG6923 | 5E−32 | 28 | 154123_at |
| 10933 | FBgn0027378 | CG6363 | 9E−76 | 39 | 154132_at |
| 9135 | FBgn0034585 | CG4030 | 9E−32 | 24 | 154147_at |
| 2107 | FBgn0036974 | CG5605 | 0 | 85 | 154148_at |
| 4580 | FBgn0037710 | CG9393 | 5E−31 | 29 | 154154_at |
| 9942 | FBgn0038463 | CG3534 | 1E−123 | 43 | 154175_at |
| 6888 | FBgn0023477 | CG2827 | 8E−44 | 60 | 154176_at |
| 7184 | FBgn0039562 | CG5520 | 0 | 60 | 154182_at |
| 5723 | FBgn0023129 | CG3705 | 4E−56 | 50 | 154184_at |
| 8841 | FBgn0025825 | CG2128 | 1E−177 | 65 | 154196_at |
| 22985 | FBgn0032745 | CG10473 | 1E−70 | 28 | 154197_at |
| 10480 | FBgn0033902 | CG8309 | 1E−94 | 47 | 154198_at |
| 3068 | FBgn0039743 | CG7946 | 3E−16 | 34 | 154202_at |
| 6206 | FBgn0014027 | CG11271 | 2E−43 | 67 | 154203_at |
| 6683 | FBgn0039141 | CG5977 | 1E−135 | 45 | 154205_at |
| 246269 | FBgn0033734 | CG8520 | 4E−95 | 44 | 154207_at |
| 10921 | FBgn0037707 | CG16788 | 3E−56 | 39 | 154209_at |
| 6045 | FBgn0020911 | CG5595 | 1E−66 | 47 | 154217_at |
| 2746 | FBgn0001098 | CG5320 | 0 | 74 | 154218_at |
| 6207 | FBgn0010265 | CG13389 | 2E−74 | 87 | 154219_at |
| 6187 | FBgn0004867 | CG5920 | 1E−118 | 82 | 154228_at |
| 38 | FBgn0029969 | CG10932 | 1E−149 | 65 | 154229_at |
| 9578 | FBgn0023081 | CG4012 | 0 | 40 | 154234_at |
| 51118 | FBgn0030063 | CG1789 | 2E−41 | 39 | 154236_at |
| 9058 | FBgn0036816 | CG3979 | 1E−107 | 37 | 154241_at |
| 3911 | FBgn0002526 | CG10236 | 0 | 32 | 154252_at |
| 5837 | FBgn0004507 | CG7254 | 0 | 73 | 154262_at |
| 1933 | FBgn0028737 | CG6341 | 7E−71 | 58 | 154264_at |
| 53354 | FBgn0011205 | CG5725 | 1E−136 | 64 | 154272_at |
| 558 | FBgn0050122 | CG30122 | 1E−53 | 29 | 154274_at |
| 79568 | FBgn0035088 | CG3776 | 0.000000000002 | 25 | 154280_at |
| 55660 | FBgn0031492 | CG3542 | 1E−177 | 43 | 154288_at |
| 6610 | FBgn0035421 | CG12034 | 3E−46 | 35 | 154300_at |
| 23592 | FBgn0034962 | CG3167 | 2E−25 | 24 | 154305_at |
| 83939 | FBgn0037135 | CG7414 | 1E−113 | 36 | 154306_at |
| 37 | FBgn0034432 | CG7461 | 0 | 57 | 154311_at |
| 9079 | FBgn0013764 | CG3924 | 1E−131 | 65 | 154317_at |
| 3727 | FBgn0001291 | CG2275 | 6E−33 | 31 | 154320_at |
| 11052 | FBgn0035872 | CG7185 | 1E−108 | 45 | 154322_at |
| 4722 | FBgn0035404 | CG12079 | 2E−89 | 64 | 154334_at |
| 8650 | FBgn0002973 | CG3779 | 2E−89 | 54 | 154346_at |
| 23192 | FBgn0031298 | CG4428 | 6E−83 | 46 | 154359_at |
| 51616 | FBgn0000617 | CG6474 | 3E−37 | 46 | 154366_at |
| 9825 | FBgn0041582 | CG4057 | 0.00000000002 | 28 | 154372_at |
| 55122 | FBgn0035773 | CG8580 | 1E−30 | 40 | 154373_at |
| 9352 | FBgn0035631 | CG5495 | 6E−81 | 49 | 154374_at |
| 55756 | FBgn0036570 | CG5222 | 0 | 51 | 154376_at |
| 81556 | FBgn0031314 | CG4785 | 5E−60 | 31 | 154378_at |
| 64786 | FBgn0031233 | CG11490 | 1E−88 | 47 | 154389_at |
| 57102 | FBgn0039589 | CG9986 | 4E−86 | 35 | 154402_at |
| 5496 | FBgn0033021 | CG10417 | 1E−99 | 38 | 154404_at |
| 2582 | FBgn0035147 | CG12030 | 1E−115 | 56 | 154410_at |
| 79882 | FBgn0028471 | CG5720 | 2E−36 | 27 | 154413_at |
| 143187 | FBgn0035156 | CG3279 | 1E−36 | 37 | 154415_at |
| 10130 | FBgn0025678 | CG5809 | 1E−148 | 60 | 154419_at |
| 84858 | FBgn0005771 | CG4491 | 7E−37 | 29 | 154420_at |
| 1456 | FBgn0011253 | CG6963 | 1E−180 | 72 | 154428_at |
| 27255 | FBgn0037240 | CG1084 | 1E−134 | 30 | 154446_at |
| 9343 | FBgn0039566 | CG4849 | 0 | 75 | 154447_at |
| 3276 | FBgn0037834 | CG6554 | 1E−145 | 65 | 154448_at |
| 23223 | FBgn0030504 | CG2691 | 1E−158 | 29 | 154451_at |
| 55207 | FBgn0037551 | CG7891 | 2E−95 | 88 | 154468_at |
| 26249 | FBgn0001301 | CG7210 | 0 | 60 | 154485_at |
| 27341 | FBgn0031764 | CG9107 | 4E−32 | 34 | 154505_at |
| 140890 | FBgn0024285 | CG4602 | 2E−68 | 35 | 154506_at |
| 55176 | FBgn0026571 | CG9539 | 0 | 91 | 154508_at |
| 63935 | FBgn0037021 | CG11399 | 1E−167 | 44 | 154518_at |
| 1496 | FBgn0010215 | CG17947 | 0 | 65 | 154527_at |
| 26355 | FBgn0036887 | CG9231 | 3E−18 | 43 | 154531_at |
| 6182 | FBgn0031450 | CG2903 | 1E−154 | 41 | 154536_at |
| 10159 | FBgn0037671 | CG8444 | 5E−27 | 26 | 154539_at |
| 123169 | FBgn0019637 | CG1433 | 1E−104 | 35 | 154540_at |
| 9963 | FBgn0037807 | CG6293 | 1E−143 | 44 | 154546_at |
| 55210 | FBgn0040237 | CG6815 | 0 | 62 | 154554_at |
| 221079 | FBgn0035866 | CG7197 | 4E−77 | 72 | 154562_at |
| 1021 | FBgn0016131 | CG5072 | 9E−76 | 46 | 154563_at |
| 5875 | FBgn0037293 | CG12007 | 4E−75 | 37 | 154565_at |
| 56954 | FBgn0037687 | CG8132 | 1E−82 | 53 | 154567_at |
| 1120 | FBgn0027842 | CG12891 | 0 | 51 | 154586_at |
| 5007 | FBgn0020626 | CG6708 | 0 | 47 | 154587_at |
| 11189 | FBgn0036379 | CG12478 | 1E−105 | 54 | 154590_at |
| 27352 | FBgn0038304 | CG12241 | 0 | 65 | 154604_at |
| 51635 | FBgn0051937 | CG31937 | 5E−45 | 34 | 154613_at |
| 10808 | FBgn0026418 | CG6603 | 0 | 44 | 154626_at |
| 9372 | FBgn0026369 | CG15667 | 1E−163 | 40 | 154629_at |
| 8500 | FBgn0031852 | CG11199 | 0 | 50 | 154634_at |
| 10651 | FBgn0036920 | CG8004 | 1E−58 | 44 | 154636_at |
| 57679 | FBgn0037116 | CG7158 | 3E−53 | 25 | 154640_at |
| 11321 | FBgn0040346 | CG3704 | 1E−92 | 48 | 154641_at |
| 2033 | FBgn0015624 | CG15319 | 0 | 49 | 154642_at |
| 8694 | FBgn0051991 | CG31991 | 1E−108 | 44 | 154661_at |
| 9931 | FBgn0036451 | CG9425 | 0 | 40 | 154668_at |
| 10652 | FBgn0029978 | CG1515 | 3E−66 | 61 | 154675_at |
| 5529 | FBgn0027492 | CG5643 | 0 | 76 | 154680_at |
| 5066 | FBgn0033466 | CG12130 | 1E−63 | 33 | 154695_at |
| 329 | FBgn0015247 | CG8293 | 1E−69 | 29 | 154697_at |
| 178 | FBgn0034618 | CG9485 | 0 | 46 | 154730_at |
| 8604 | FBgn0028646 | CG2139 | 0 | 58 | 154731_at |
| 25839 | FBgn0032258 | CG7456 | 1E−157 | 38 | 154734_at |
| 10257 | FBgn0051793 | CG31793 | 0 | 45 | 154740_at |
| 10096 | FBgn0011744 | CG7558 | 0 | 80 | 154742_at |
| 9517 | FBgn0002524 | CG4162 | 1E−163 | 56 | 154751_at |
| 51701 | FBgn0011817 | CG7892 | 0 | 76 | 154754_at |
| 25978 | FBgn0035589 | CG4618 | 7E−49 | 52 | 154760_at |
| 5236 | FBgn0003076 | CG5165 | 0 | 59 | 154763_at |
| 84640 | FBgn0033738 | CG8830 | 2E−67 | 27 | 154764_at |
| 3991 | FBgn0034491 | CG11055 | 9E−91 | 37 | 154767_at |
| 84662 | FBgn0033782 | CG3850 | 3E−58 | 58 | 154772_at |
| 51809 | FBgn0030930 | CG6394 | 1E−135 | 46 | 154775_at |
| 64682 | FBgn0030639 | CG9198 | 5E−80 | 27 | 154789_at |
| 80207 | FBgn0039126 | CG13601 | 5E−30 | 47 | 154797_at |
| 9924 | FBgn0033352 | CG8232 | 0 | 41 | 154804_at |
| 4904 | FBgn0022959 | CG5654 | 5E−48 | 39 | 154807_at |
| 8985 | FBgn0036147 | CG6199 | 0 | 46 | 154810_at |
| 2643 | FBgn0003162 | CG9441 | 5E−79 | 75 | 154812_at |
| 81608 | FBgn0037255 | CG1078 | 9E−44 | 24 | 154839_at |
| 26100 | FBgn0035850 | CG7986 | 1E−119 | 61 | 154843_at |
| 79944 | FBgn0032729 | CG10639 | 1E−135 | 55 | 154852_at |
| 51128 | FBgn0038947 | CG7073 | 1E−79 | 70 | 154858_at |
| 667 | FBgn0013733 | CG18076 | 0 | 39 | 154862_at |
| 26575 | FBgn0028743 | CG5036 | 3E−66 | 56 | 154885_at |
| 157807 | FBgn0036289 | CG10657 | 9E−35 | 34 | 154890_at |
| 7283 | FBgn0004176 | CG3157 | 0 | 77 | 154893_at |
| 55023 | CG31132 | CG31132 | 0 | 39 | 154904_at |
| 6426 | FBgn0040284 | CG6987 | 9E−81 | 63 | 154911_at |
| 27291 | FBgn0035388 | CG2162 | 2E−50 | 50 | 154920_at |
| 79796 | FBgn0039293 | CG11851 | 1E−148 | 45 | 154932_at |
| 2874 | FBgn0033122 | CG17002 | 0.000000000000006 | 26 | 154950_at |
| 79183 | FBgn0032783 | CG10237 | 3E−39 | 34 | 154963_at |
| 56259 | FBgn0037644 | CG11964 | 1E−177 | 55 | 154969_at |
| 23154 | FBgn0037447 | CG2330 | 4E−81 | 28 | 154993_at |
| 89845 | FBgn0032018 | CG7806 | 0 | 40 | 155000_at |
| 10210 | FBgn0034410 | CG15104 | 1E−41 | 24 | 155009_at |
| 10013 | FBgn0026428 | CG6170 | 0 | 44 | 155023_at |
| 3998 | FBgn0020254 | CG6822 | 1E−113 | 41 | 155025_at |
| 10598 | FBgn0032961 | CG1416 | 7E−77 | 42 | 155036_at |
| 5573 | FBgn0000275 | CG3263 | 1E−150 | 80 | 155037_at |
| 6804 | CG31136 | CG31136 | 1E−112 | 70 | 155041_at |
| 9615 | FBgn0037219 | CG18143 | 8E−95 | 43 | 155054_at |
| 55633 | FBgn0038855 | CG5745 | 1E−123 | 45 | 155071_at |
| 152559 | FBgn0038256 | CG7530 | 2E−57 | 38 | 155074_at |
| 7163 | FBgn0034345 | CG5174 | 3E−22 | 38 | 155076_at |
| 23597 | FBgn0039854 | CG1635 | 6E−72 | 36 | 155089_at |
| 54931 | FBgn0034351 | CG5190 | 2E−38 | 32 | 155092_at |
| 438 | FBgn0037362 | CG2179 | 1E−100 | 34 | 155114_at |
| 55833 | FBgn0033253 | CG8715 | 6E−38 | 27 | 155119_at |
| 3190 | FBgn0015907 | CG13425 | 3E−40 | 33 | 155123_at |
| 124245 | FBgn0029941 | CG1677 | 2E−43 | 26 | 155128_at |
| 8189 | FBgn0037371 | CG2097 | 0 | 35 | 155136_at |
| 10197 | FBgn0029133 | CG1591 | 3E−68 | 48 | 155140_at |
| 2271 | FBgn0029889 | CG4094 | 0 | 76 | 155145_at |
| 9986 | FBgn0029121 | CG4852 | 2E−37 | 40 | 155164_at |

1. A method to treat, prevent or ameliorate pathological conditions associated with dysregulation of the insulin signaling pathway (ISP) comprising administering to a subject in need thereof an effective amount of a modulator of a protein selected from the group consisting of those disclosed in Table 13 or 25.

2. The method of claim 1, wherein said condition is Type II diabetes.

3. The method of claim 1, wherein said condition is the Type A syndrome of insulin resistance.

4. The method of claim 1, wherein said modulator inhibits the biochemical function of said protein in said subject.

5. The method of claim 4, wherein said modulator comprises one or more antibodies to said protein, or fragments thereof, wherein said antibodies or fragments thereof can inhibit the biochemical function of said protein in said subject.

6. The method of claim 1, wherein said modulator enhances the biochemical function of said protein in said subject.

7. The method of claim 1, wherein said modulator inhibits gene expression of said protein in said subject.

8. The method of claim 7, wherein said modulator comprises any one or more substances selected from the group consisting of antisense oligonucleotides, triple-helix DNA, ribozymes, RNA aptamers, siRNA, single- and double-stranded RNA wherein said substances are designed to inhibit gene expression of said protein.

9. The method of claim 1, wherein said modulator enhances the gene expression of said protein in said subject.

10. A method to treat, prevent or ameliorate pathological conditions associated with dysregulation of the insulin signaling pathway comprising administering to a subject in need thereof a pharmaceutical composition comprising an effective amount of a modulator of a protein selected from the group consisting of those disclosed in Table 13 or 25.

11. The method of claim 10, wherein said condition is Type II diabetes.

12. The method of claim 10, wherein said condition is the Type A syndrome of insulin resistance.

13. The method of claim 10, wherein said modulator inhibits the biochemical function of said protein in said subject.

14. The method of claim 13, wherein said modulator comprises one or more antibodies to said protein, or fragments thereof, wherein said antibodies or fragments thereof can inhibit the biochemical function of said protein.

15. The method of claim 10, wherein said modulator enhances the biochemical function of said protein in said subject

16. The method of claim 10, wherein said modulator inhibits gene expression of said protein in said subject.

17. The method of claim 16, wherein said modulator comprises any one or more substances selected from the group consisting of antisense oligonucleotides, triple-helix DNA, ribozymes, RNA aptamers, siRNA, single- and double-stranded RNA wherein said substances are designed to inhibit gene expression of said protein.

18. The method of claim 10, wherein said modulator enhances gene expression of said protein in said subject.

19. A method to identify modulators useful to treat, prevent or ameliorate pathological conditions associated with dysregulation of the ISP comprising assaying for the ability of a candidate modulator to modulate the biochemical function of a protein selected from the group consisting of those disclosed in Table 13 or 25.

20. The method of claim 19, wherein said method further comprises assaying for the ability of an identified modulator to reverse the pathological effects observed in animal models of said conditions.

21. The method of claim 19, wherein said method further comprises assaying for the ability of an identified modulator to reverse the pathological effects observed in clinical studies with subjects with said conditions.

22. The method according to claim 19, wherein said condition is Type II diabetes.

23. The method according to claim 19, wherein said condition is the Type A syndrome of insulin resistance.

24. A method to identify modulators useful to treat, prevent or ameliorate pathological conditions associated with dysregulation of the ISP comprising assaying for the ability of a candidate modulator to modulate gene expression of a protein selected from the group consisting of those disclosed in Table 13 or 25.

25. The method according to claim 24, wherein said method further comprises assaying for the ability of an identified inhibitory modulator to reverse the pathological effects observed in animal models of said condition.

26. The method according to claim 24, wherein said method further comprises assaying for the ability of an identified inhibitory modulator to reverse the pathological effects observed in clinical studies with subjects with said condition.

27. The method according to claim 24, wherein said condition is Type II diabetes.

28. The method according to claim 24, wherein said condition is the Type A syndrome of insulin resistance.

29. A pharmaceutical composition comprising a modulator to a protein selected from the group consisting of those disclosed in Table 13 or 25 in an amount effective to treat, prevent or ameliorate pathological conditions associated with dysregulation of the ISP in a subject in need thereof.

30. The pharmaceutical composition according to claim 29, wherein said condition is Type II diabetes.

31. The pharmaceutical composition according to claim 29, wherein said condition is the Type A syndrome of insulin resistance.

32. The pharmaceutical composition according to claim 29, wherein said modulator inhibits the biochemical function of said protein.

33. The pharmaceutical composition of claim 29, wherein said modulator comprises one or more antibodies to said protein, or fragments thereof, wherein said antibodies or fragments thereof can inhibit the biochemical function of said protein.

34. The pharmaceutical composition according to claim 29, wherein said modulator enhances the biochemical function of said protein.

35. The pharmaceutical composition according to claim 29, wherein said modulator inhibits gene expression of said protein.

36. The pharmaceutical composition of claim 29, wherein said modulator comprises any one or more substances selected from the group consisting of antisense oligonucleotides, triple helix DNA, ribozymes, RNA aptamer, siRNA, single- and double-stranded RNA wherein said substances are designed to inhibit gene expression of said protein.

37. The pharmaceutical composition according to claim 25, wherein said modulator enhances gene expression of said protein.

38. A method to diagnose subjects suffering from pathological conditions associated with dysregulation of the ISP who may be suitable candidates for treatment with modulators to a protein selected from the group consisting of those disclosed in Table 13, comprising assaying mRNA levels of any one or more of said proteins in a biological sample from said subject wherein subjects with altered levels compared to controls would be suitable candidates for modulator treatment.

39. A method to diagnose subjects suffering from pathological conditions associated with dysregulation of the ISP who may be suitable candidates for treatment with modulators to a protein selected from the group consisting of those disclosed in Table 13, comprising detecting levels of any one or more of said proteins in a biological sample from said subject wherein subjects with altered levels compared to controls would be suitable candidates for modulator treatment.

40. A method to treat, prevent or ameliorate a pathological condition associated with dysregulation of the ISP comprising:
(a) assaying for mRNA levels of a protein selected from the group consisting of those disclosed in Tables 13 and 25 in a subject; and
(b) administering to a subject with altered levels of mRNA of said protein compared to controls a modulator to said protein in an amount sufficient to treat, prevent or ameliorate the pathological effects of said condition.

41. The method of claim 40, wherein said condition is Type II diabetes.

42. The method of claim 40, wherein said condition is the Type A syndrome of insulin resistance.

43. The method of claim 40, wherein said modulator enhances the gene expression of said protein.

44. The method of claim 40, wherein said modulator inhibits the gene expression of said protein.

45. A method to treat, prevent or ameliorate a pathological condition associated with dysregulation of the ISP comprising:
(a) assaying for levels of a protein selected from the group consisting of those disclosed in Table 13 or 25 in a subject; and
(b) administering to a subject with altered levels of said protein compared to controls a modulator to said protein in an amount sufficient to treat, prevent or ameliorate the pathological effects of said condition.

46. The method of claim 45, wherein said condition is Type II diabetes.

47. The method of claim 45, wherein said condition is the Type A syndrome of insulin resistance.

48. The method of claim 45, wherein said modulator enhances the biochemical function of said protein.

49. The method of claim 45, wherein said modulator inhibits the biochemical function of said protein.

50. A diagnostic kit for detecting mRNA levels of a protein selected from the group consisting of those disclosed in Tables 13 and 25 in a biological sample, said kit comprising:
(a) a polynucleotide of a polypeptide set forth in Table 13 or 25 or a fragment thereof;
(b) a nucleotide sequence complementary to that of (a);
(c) a polypeptide of Table 13 or 25 of the present invention encoded by the polynucleotide of (a);
(d) an antibody to the polypeptide of (c); and
(e) an RNAi sequence complementary to that of (a),
wherein components (a), (b), (c), (d) or (e may comprise a substantial component.

51. A diagnostic kit for detecting levels of a protein selected from the group consisting of those disclosed in Table 13 or 25 in a biological sample, said kit comprising:
(a) a polynucleotide of a polypeptide set forth in Table 13 or 25 or a fragment thereof;
(b) a nucleotide sequence complementary to that of (a);
(c) a polypeptide of Table 13 or 25 of the present invention encoded by the polynucleotide of (a);
(d) an antibody to the polypeptide of (c); and
(e) an RNAi sequence complementary to that of (a),
wherein components (a), (b), (c), (d) or (e) may comprise a substantial component.

52. A method to identify genetic modifiers of the insulin signaling pathway, said method comprising:
(a) providing a transgenic fly whose genome comprises a DNA sequence encoding a polypeptide comprising Dp110^{D954A}, said DNA sequence operably linked to a tissue specific control sequence, and expressing said DNA sequence, wherein expression of said DNA sequence results in said fly displaying a transgenic phenotype;
(b) crossing said transgenic fly with a fly containing a mutation in a known or predicted gene; and
(c) screening progeny of said crosses for flies that carry said DNA sequence and said mutation and display modified expression of the transgenic phenotype as compared to controls.

53. The method of claim 52, wherein said DNA sequence encodes Dp110^{D954A} and wherein said tissue specific expression control sequence comprises the eye specific enhancer (ey-Gal4).

54. The method of claim 53, wherein expression of said DNA sequence results in said fly displaying the “small eye” phenotype.

55. A method to identify targets for the development of therapeutics to treat, prevent or ameliorate pathological conditions associated with dysregulation of the ISP said method comprising identifying the human homologs of the genetic modifiers identified according to the method of claim 52.

56. The method of claim 55, wherein said condition is Type II diabetes.

57. The method of claim 55, wherein said condition is the Type A syndrome of insulin resistance.
